(19) United States
(12) Patent Application Publication
(10) Pub. No.: US 2022/0282211 A1
(43) Pub. Date: Sep. 8, 2022

(54) ANTIBODY PRODUCING MICROFLUIDIC DEVICES

(71) Applicant: EMULATE, INC., Boston, MA (US)

(72) Inventors: Alicia Stark, Boston, MA (US); Brar Gurpreet, Boston, MA (US)

(21) Appl. No.: 17/746,097

(22) Filed: May 17, 2022

Related U.S. Application Data
(63) Continuation of application No. PCT/US2020/061579, filed on Nov. 20, 2020.
(60) Provisional application No. 62/938,755, filed on Nov. 21, 2019.

(51) Int. Cl. C12N 5/0781; C12M 3/06; C07K 16/00

(52) U.S. Cl. CPC C12N 5/0635; C12M 23/16; C07K 16/00; C12N 2501/2302; C12N 2501/2321; C12N 2501/25; C12N 2501/2306; C12N 2501/24; C07K 2317/14

(57) ABSTRACT

The present invention relates to fluidic systems for producing IgG antibodies from co-cultures of white blood cells. In some embodiments, a microfluidic device containing co-cultures of an autologous whole peripheral white blood cell population including B cells, are used for providing antigen specific IgG antibody production from differentiating B cells (plasma cells). More specifically, high levels of IgM and IgG classes of antibodies are harvested from fluids flowing through the device. In some embodiments, IgG is produced during activation in the presence of antigen, including but not limited to therapeutic immunogenic compounds, e.g. engineered antibodies, vaccines, etc. In some embodiments, such co-cultures are further exposed to drug compounds e.g. for preclinical safety testing and individualized personal drug responses. In some embodiments, such antibody producing microfluidic devices are contemplated for use in companion diagnostic and complementary assays.

### FIELD OF THE DISCLOSURE

The present invention relates to fluidic systems for producing IgG antibodies from co-cultures of white blood cells. In some embodiments, a microfluidic device containing co-cultures of an autologous whole peripheral white blood cell population including B cells, are used for providing antigen specific IgG antibody production from differentiating B cells (plasma cells). More specifically, high levels of IgM and IgG classes of antibodies are harvested from fluids flowing through the device. In some embodiments, IgG is produced during activation in the presence of antigen, including but not limited to therapeutic immunogenic compounds, e.g. engineered antibodies, vaccines, etc. In some embodiments, such co-cultures are further exposed to drug compounds e.g. for preclinical safety testing and individualized personal drug responses. In some embodiments, such antibody producing microfluidic devices are contemplated for use in companion diagnostic and complementary assays.

### BACKGROUND

Cutting edge treatments of diseases include administration of biopharmaceuticals, e.g. humanized monoclonal antibodies (including autoimmune diseases) and fusion proteins (including vaccines) directed to in vivo target molecules. As one biopharmaceutical example, the use of humanized monoclonal antibodies for reducing/blocking inflammatory actions of disease related cytokines. For many sufferers of extensive debilitating, disfiguring and painful autoimmune conditions, humanized antibody treatments provide a welcome relief not found with other types of pharmaceutical treatments.

Commercially produced humanized recombinant monoclonal antibody pharmaceuticals are provided through in vitro processes using a combination of engineered antibody DNA, viral expression molecules and cell lines, often cancer-like cell lines. While the pharmaceutical product undergoes extensive in vitro viral, microbial, physico-chemical, biological and immunological testing (i.e. pharmacodynamics) for ensuring patient safety to the pharmaceutical itself, there are few in vivo preclinical safety tests available for identifying adverse effects in humans. Even primates, e.g. cynomolgus monkeys, in addition to other preclinical test animals, such as engineered or specially bred mice, rats, guinea pigs, rabbits and dogs, have enough significant physiological and/or immunological differences to preclude obtaining accurate information during preclinical testing for observing adverse human responses.

In fact, as one example, after extensive in vitro and in vivo animal preclinical testing at least one humanized antibody cleared by animal testing for human clinical trials, surprisingly produced adverse reactions in people. Many of the adverse reactions in people were related to effects of the presence of endogenous antibodies cross-reacting to the humanized antibodies.

Because human:humanized antibodies/fusion proteins are foreign to the test animals, test animals (unless engineered otherwise) will develop immune responses, e.g. antibody reactions, against the biopharmaceutical (Bugelski and Treacy, 2004). Moreover, development of antibody responses to biopharmaceuticals in animals in some cases were later associated with underestimating human safety to the pharmaceutical humanized antibody when administer to humans. Conversely, immune responses in animals that may indicate potential human antibody related toxicities, such as hypersensitivity, anaphylaxis, serum sickness or immune complex disease, may result in an overestimation of the toxicity of the biopharmaceutical thus potentially missing an effective human therapeutic.

Overall, immunological reactions in animals can not be used to accurately predict potential immunological reactions in humans (Bugelski and Treacy, 2004; Ponce et al., 2009). See, for a review, Martin and Bugelski, 2012.

Therefore, there is a need for more accurate preclinical in vitro tests directly applicable to humans for identifying adverse antibody responses to engineered biopharmaceuticals.

### SUMMARY OF THE INVENTION

The present invention relates to fluidic systems for producing IgG antibodies from co-cultures of white blood cells. In some embodiments, a microfluidic device containing a co-cultures of an autologous whole peripheral white blood cell population including B cells, are used for providing antigen specific IgG antibody production from differentiating B cells (plasma cells). More specifically, high levels of IgM and IgG classes of antibodies are harvested from fluids flowing through the device. In some embodiments, IgG is produced during activation in the presence of antigen, including but not limited to therapeutic immunogenic compounds, e.g. engineered antibodies, vaccines, vaccine candidates, etc. In some embodiments, such co-cultures are further exposed to drug compounds e.g. for preclinical safety testing and individualized personal drug responses. In some embodiments, such antibody producing microfluidic devices are contemplated for use in companion diagnostic and complementary assays.

Previous Organs-on-Chips technology is undergoing adaptation for creating a new living system as an antibody producing Lymph Node Chip, i.e. AB-Lymph Node Chip, emulating human biology in relation to mimicking endogenous antibody production. Initial results using this new technology is already fundamentally changing how our understanding of biology related to immune responses by enabling the production of data, i.e. up to high levels of immunoglobulin production in ranges of immunoglobulin amounts that may be measured in vivo in serum, contemplated for use in predicting how diseases, medicines, chemicals, and foods affect human health. Moreover, embodiments of such data may be obtained under certain conditions for providing predictions applied to individuals, selected populations or populations in general, in particular for populations intended as recipients of biologic treatments, drug treatments and vaccines.

Antibody producing microfluidic devices as described herein, may be seeded with white blood cell populations derived from any mammalian for providing specifies specific antibodies. Moreover, methods for providing antibodies from white blood cell populations are not limited to use in microfluidic chips. Indeed, methods described herein may be used in plate cultures, including Petri dishes, multi-well plates, etc., bioreactors and scaled up bioreactors for large scale antibody production, etc.

Exemplary antibody producing microfluidic devices as described herein, undergo up to three phases of antibody production, each using a different media, e.g. activation medium, differentiation medium, and maintenance medium. However, it is not meant to limit the number of phases, such that antibody producing devices may undergo activation without any further phases. In other embodiments, antibody producing devices may undergo activation and differentiation. In yet other embodiments, antibody producing devices may undergo activation, differentiation and maintenance. In yet other embodiments, antibody producing devices may undergo additional phases, such as re-stimulation, differentiation, etc. In yet other embodiments, antibody producing devices may undergo additional phases, such as phases for mimicking follicular T cell development for inducing and/or supporting B cells of any developmental or differentiation stage.

In one embodiment, the present invention provides a microfluidic device comprising a space located in between an inlet and an outlet, said space comprising a human cell population comprising B cells, said cells suspended in a hydrogel precursor, said B cells exposed to a B cell activation medium, comprising a test substance and one or more activation associated molecules selected from the group consisting of IL-2, IL-21 and soluble CD40L molecules. In one embodiment, said B cell activation medium contains only two activation associated molecules (along with the test substance), namely IL-21 and soluble CD40L molecules (i.e. it does not contain IL-2). In one embodiment, said soluble CD40L is provided by CD40L expressing feeder cells.

In one embodiment, the present invention provides a method of activating B cells, comprising, a) providing; i) a microfluidic device comprising a space located in between an inlet and an outlet; ii) a human cell population comprising B cells, said cells suspended in a gel precursor (including but not limited to a hydrogel precursor); and iii) a B cell activation medium, comprising a test substance and one or more activation associated molecules selected from the group consisting of IL-2, IL-21 and soluble CD40L molecules; b) introducing said gel precursor (e.g. hydrogel precursor) into said space; c) treating said gel precursor (e.g. hydrogel precursor) under conditions so as to at least partially solidify said gel or hydrogel; and d) flowing said B cell activation medium under conditions such that said B cells are exposed to said medium, wherein at least a portion of said B cells are activated to produce antibody. In one embodiment, said method further comprising: e) collecting effluent from said outlet. In one embodiment, said method further comprising: f) measuring the amount of antibodies in said effluent. In one embodiment, said step e) occurs 1 to 10 days after step d). In one embodiment, said B cell activation medium contains only two activation associated molecules (along with the test substance), namely IL-21 and soluble CD40L molecules (i.e. it does not contain IL-2). In one embodiment, said soluble CD40L is provided by CD40L expressing feeder cells. In one embodiment, said test substance is selected from the group consisting of live bacteria, inactivated bacteria, bacterial spores, live virus, inactivated virus, live fungi, inactivated fungi and fungal spores. In one embodiment, said test substance is selected from a drug, a vaccine (or vaccine candidate), a cosmetic and a food substance. In one embodiment, said test substance is an antigen selected from the group consisting of a bacterial antigen, a viral antigen and a fungal antigen. In one embodiment, said antibodies comprise immunoglobulin M (IgM). In one embodiment, said IgM is at a concentration of up to 40,000 ng/mL. In one embodiment, said antibodies comprise immunoglobulin G (IgG). In one embodiment, said IgG is at a concentration of up to 380,000 ng/mL. In one embodiment, said B cells are exposed to said activation medium for 2-4 days. In one embodiment, at least a portion of said activated B cells differentiate. In one embodiment, said differentiating B cells comprise plasmablasts and plasma cells. In one embodiment, said method further comprising, after exposing B cells to said activation medium, exposing said B cells to a maintenance medium, said maintenance medium lacking IL-2 and soluble CD40L molecules. In one embodiment, said maintenance medium exposure ranges from 2-5 days. In one embodiment, said maintenance medium comprises molecules selected from the group consisting of IL-6, IL-21, and IFN-alpha. In one embodiment, said B cells of step a) comprise memory B cells and naive B cells. In one embodiment, said hydrogel comprises a mixture of Engelbreth-Holm-Swarm (EHS) mouse tumor basement membrane proteins. In one embodiment, said hydrogel comprises a Matrigel® protein mixture. In one embodiment, said hydrogel is a mixture of bovine collagen I proteins and a mixture of basement membrane extracted from Engelbreth-Holm-Swarm (EHS) mouse sarcoma tumors. In one embodiment, said hydrogel is a mixture of bovine collagen I proteins and a Matrigel® protein mixture. In one embodiment, said human cell population is a Peripheral blood mononuclear cell (PBMC) population. It is not intended to limit said PBMC population to any one or more cell type. Indeed PBMCs intended for use include but not limited to an isolated (e.g. from red blood cells) total/whole PBMC population, and purified populations of cells derived from the PBMCs, such as memory B cells, naive B cells, activated B cells, lymphocytes, B cells and T cells, monocytes, etc. In one embodiment, said human cell population is an isolated tonsil white blood cell population. In one embodiment, said human cell population is an isolated lymph node white blood cell population. In one embodiment, said human cell population is a purified population of CD19+CD27+ B cells. In one embodiment, said human cell population is a mixture of a purified population of CD19+CD27+ B cells and CD3+CD4+ T helper cells. In one embodiment, said human cell population is a mixture of a purified population of CD19+CD27+ B cells and CD3+CD4+CXCR5(C-X-C Motif Chemokine Receptor 5)+ICOS(inducible T cell co-stimulator)+PD-1(programmed cell death-1)^{hi} T helper follicular cells. In one embodiment, said flowing of said differentiation media is continuous flowing. In one embodiment, said device fits into and is fluidically connected to a culture module that in turns fits into a perfusion manifold device. In one embodiment, said test substance is an antibody or antibody fragment. In one embodiment, said antibody is an anti-human-antibody or antibody fragment.

It is not intended to limit the type of CDL stimulation. Indeed, in some embodiments, CD40Ligand stimulation may be provided by any one or more of soluble CD40L, dimer of CD40L, trimer of CD40L, recombinant human CD40L, histidine tagged CD40L, cells expressing cell surface CD40L, etc.

The present invention provides an extracellular matrix composition comprising bovine collagen I proteins.

The present invention provides an extracellular matrix composition comprising bovine collagen I proteins and a mixture of Engelbreth-Holm-Swarm (EHS) mouse sarcoma cell basement membrane proteins.

The present invention provides an extracellular matrix composition comprising bovine collagen I proteins and a mixture of Engelbreth-Holm-Swarm (EHS) mouse tumor basement membrane proteins.

The present invention provides an extracellular matrix composition comprising bovine collagen I proteins and a mixture of Matrigel® proteins.

The present invention provides an extracellular matrix composition consisting of bovine collagen I proteins.

The present invention provides an extracellular matrix composition consisting of bovine collagen I proteins and a mixture of Engelbreth-Holm-Swarm (EHS) mouse sarcoma cell basement membrane proteins.

The present invention provides an extracellular matrix composition consisting of bovine collagen I proteins and a mixture of Engelbreth-Holm-Swarm (EHS) mouse tumor basement membrane proteins.

The present invention provides an extracellular matrix composition comprising bovine collagen I proteins and a mixture of Matrigel® proteins.

In one embodiment, the present invention provides a method of activating B cells, comprising, a) providing; i) a microfluidic device comprising a space located in between an inlet and an outlet; ii) a human cell population comprising B cells, said cells suspended in a hydrogel precursor; and iii) a B cell activation medium, comprising a test substance and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, wherein at least a portion of said B cells are activated to produce antibody. In one embodiment, said B cell activation medium contains both activation associated molecules (along with the test substance), namely IL-21 and soluble CD40L molecules (i.e. it does not contain IL-2). In one embodiment, said soluble CD40L is provided by CD40L expressing feeder cells.

In one embodiment, the present invention provides a method of using antigen for activating B cells, comprising, a) providing; i) a microfluidic device comprising a space located in between an inlet and an outlet; ii) a human cell population comprising B cells, said cells suspended in a hydrogel precursor; and iii) a B cell activation medium, comprising a tetanus toxoid (TT) antigen and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, wherein at least a portion of said B cells are activated to produce antibody. In one embodiment, said B cell activation medium contains both activation associated molecules (along with the TT), namely IL-21 and soluble CD40L molecules (i.e. it does not contain IL-2). In one embodiment, said soluble CD40L is provided by CD40L expressing feeder cells.

In one embodiment, the present invention provides a method of using antigen for activating B cells, comprising, a) providing; i) a microfluidic device comprising a space located in between an inlet and an outlet; ii) a human cell population comprising B cells, said cells suspended in a hydrogel precursor; and iii) a B cell activation medium, comprising a test substance and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, wherein at least a portion of said B cells are activated to produce germinal center-like clusters. In one embodiment, said B cell activation medium contains both activation associated molecules (along with the test substance), namely IL-21 and soluble CD40L molecules (i.e. it does not contain IL-2). In one embodiment, said soluble CD40L is provided by CD40L expressing feeder cells.

In one embodiment, the present invention provides a method of providing a gradient within a microfluidic device, comprising, a) providing; i) a microfluidic device comprising a space located in between an inlet and an outlet; ii) a human cell population comprising B cells, said cells suspended in a hydrogel precursor; and iii) a B cell medium, comprising at least one chemokine, and one or more activation associated molecules selected from the group consisting of IL-2, IL-21 and soluble CD40L molecules; b) introducing said hydrogel precursor into said space; c) treating said hydrogel precursor under conditions so as to at least partially solidify said hydrogel; and d) flowing said B cell medium under conditions such that said B cells are exposed to said medium, wherein said chemokine in said medium form gradients having lower to higher concentration levels with said hydrogel, wherein at least a portion of said B cells are located in one level. In one embodiment, said B cell medium contains only two activation associated molecules (along with the chemokine), namely IL-21 and soluble CD40L molecules (i.e. it does not contain IL-2). In one embodiment, said soluble CD40L is provided by CD40L expressing feeder cells.

In one embodiment, the present invention provides a method of providing apposing gradients within a microfluidic device, comprising, a) providing; i) a microfluidic device comprising a space located in between an inlet and an outlet; ii) a human cell population comprising B cells, said cells suspended in a hydrogel precursor; and iii) a first B cell medium, comprising at least one chemokine, and one or more activation associated molecules selected from the group consisting of IL-2, IL-21 and soluble CD40L molecules; vi) a second B cell medium, comprising at least one chemokine that is different than in said first B cell medium, and one or more activation associated molecules selected from the group consisting of IL-2, IL-21 and soluble CD40L molecules; b) introducing said hydrogel precursor into said space; c) treating said hydrogel precursor under conditions so as to at least partially solidify said hydrogel; and d) flowing said B cell medium under conditions such that said B cells are exposed to said medium, wherein said chemokines in said medium form gradients having apposing areas of lower to higher concentration levels with said hydrogel, wherein at least a portion of said B cells located in one level are stimulated to migrate into a different level in response to at least one of said chemokine gradients. In one embodiment, said B cell medium contains only two activation associated molecules (along with the chemokine), namely IL-21 and soluble CD40L molecules (i.e. it does not contain IL-2). In one embodiment, said soluble CD40L is provided by CD40L expressing feeder cells.

In one embodiment, the present invention provides a method of inducing B cell migration within a microfluidic device, comprising, a) providing; i) a microfluidic device comprising a space located in between an inlet and an outlet; ii) a human cell population comprising B cells, said cells suspended in a hydrogel precursor; and iii) a B cell medium, comprising at least one chemokine, and one or more activation associated molecules selected from the group consisting of IL-2 and soluble CD40L molecules; b) introducing said hydrogel precursor into said space; c) treating said hydrogel precursor under conditions so as to at least partially solidify said hydrogel; and d) flowing said B cell medium under conditions such that said B cells are exposed to said medium, wherein said chemokines in said medium form areas having lower to higher levels of concentration with said hydrogel, wherein at least a portion of said B cells located in one or more levels are stimulated to migrate into a different level in response to said substance. In one embodiment, said B cell medium contains both activation associated molecules (along with the chemokine), namely IL-21 and soluble CD40L molecules (i.e. it does not contain IL-2). In one embodiment, said soluble CD40L is provided by CD40L expressing feeder cells.

In one embodiment, the present invention provides a method of providing apposing gradients within a microfluidic device, comprising, a) providing; i) a microfluidic device comprising a space located in between an inlet and an outlet; ii) a human cell population comprising B cells, said cells suspended in a hydrogel precursor; and iii) a first B cell medium, comprising at least one chemokine, and one or more activation associated molecules selected from the group consisting of IL-2, IL-21 and soluble CD40L molecules; vi) a second B cell medium, comprising at least one chemokine that is different than in said first B cell medium, and one or more activation associated molecules selected from the group consisting of IL-2, IL-21 and soluble CD40L molecules; b) introducing said hydrogel precursor into said space; c) treating said hydrogel precursor under conditions so as to at least partially solidify said hydrogel; and d) flowing said B cell medium under conditions such that said B cells are exposed to said medium, wherein said chemokines in said medium form areas having apposing lower to higher levels of concentration with said hydrogel, wherein at least a portion of said B cells are located in one or more levels, wherein at least a portion of said B cells located in one or more levels are stimulated to migrate into a different level in response to said substance. In one embodiment, said B cell medium contains only two activation associated molecules (along with the chemokine), namely IL-21 and soluble CD40L molecules (i.e. it does not contain IL-2). In one embodiment, said soluble CD40L is provided by CD40L expressing feeder cells.

In one embodiment, the present invention provides a method for evaluating an engineered antibody, comprising, a) providing; i) a microfluidic device comprising a space located in between an inlet and an outlet; ii) a human cell population comprising B cells, said cells suspended in a hydrogel precursor; and iii) a B cell activation medium, comprising an engineered antibody, selected from the group consisting of bevacizumab and adalimumab, and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, wherein at least a portion of said B cells are activated to produce antibody; e) collecting effluent from said outlet; and f) measuring characteristics of antibodies selected from the group consisting of: amount, isotope, subclasses and affinities of antibodies for said engineered antibodies in said effluent, wherein said step e) occurs 1 to 10 days after step d). In one embodiment, said B cell activation medium contains only two activation associated molecules (along with the engineered antibody), namely IL-21 and soluble CD40L molecules (i.e. it does not contain IL-2). In one embodiment, said soluble CD40L is provided by CD40L expressing feeder cells. In one embodiment, said measuring indicates an antigenic response to said test substance. In one embodiment, said measuring indicates a lack of response to said test substance. In one embodiment, said further comprising, after exposing B cells to said activation medium, exposing said B cells to a differentiation medium, said differentiation medium lacking soluble CD40L molecules and including IL-2. In one embodiment, said measuring after exposing said B cells to said differentiation medium indicates an antigenic response to said test substance. In one embodiment, said measuring after exposing said B cells to said differentiation medium indicates a lack of response to said test substance.

In one embodiment, the present invention provides a method for evaluating an engineered antibody, comprising, a) providing; i) at least 30 microfluidic devices comprising a space located in between an inlet and an outlet; ii) at least 30 different human cell populations comprising B cells, said cells suspended in a hydrogel precursor; and iii) a B cell activation medium, comprising an engineered antibody, wherein said engineered antibody is selected from the group consisting of bevacizumab and adalimumab, and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, wherein at least a portion of said B cells are activated to produce antibody; e) collecting effluent from said outlet; f) measuring characteristics of antibodies selected from the group consisting of: amount, isotope and subclasses and affinities of antibodies for said engineered antibodies in said effluent, wherein said step e) occurs 1 to 10 days after step d). In one embodiment, said B cell activation medium contains both activation associated molecules (along with the engineered antibody), namely IL-21 and soluble CD40L molecules (i.e. it does not contain IL-2). In one embodiment, said soluble CD40L is provided by CD40L expressing feeder cells. In one embodiment, said measuring indicates an antigenic response to said test substance. In one embodiment, said measuring indicates a lack of response to said test substance. In one embodiment, said evaluation indicates that at least one individual patient tested is at risk of an adverse reaction if administered said test substance. In one embodiment, said different patients represent a subpopulation of patients and said evaluation indicates that said subpopulation is at risk of an adverse reaction if administered said test substance. In one embodiment, said evaluation indicates that said method is of use as a complementary assay. In one embodiment, said evaluation shows a variability in response between said patients indicating a need for developing said method as a companion diagnostic assay. In one embodiment, said determination response from at least one patient is further evaluated for determining whether or not to administer said test substance to said responder. In one embodiment, said determination to administer said test substance includes said evaluation for determining a dosage for said subject.

In one embodiment, the present invention provides a method for a germinal center formation assay, comprising, a) providing; i) a microfluidic device comprising a space located in between an inlet and an outlet; ii) a human cell population comprising B cells, said cells suspended in a hydrogel precursor; and iii) a B cell activation medium, comprising an engineered antibody, selected from the group consisting of bevacizumab and adalimumab, and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, for observing formation of germinal center-like (GC) clusters of cells for determining whether said human's cells are responding to said engineered antibody. In one embodiment, said B cell activation medium contains both activation associated molecules (along with the engineered antibody), namely IL-21 and soluble CD40L molecules (and it does not contain IL-2). In one embodiment, said soluble CD40L is provided by CD40L expressing feeder cells.

In one embodiment, the present invention provides a method for evaluating formation of germinal center-like (GC) clusters of cells, comprising, a) providing; i) a microfluidic device comprising a space located in between an inlet and an outlet; ii) a human cell population comprising B cells, said cells suspended in a hydrogel precursor; and iii) a B cell activation medium, comprising a test substance and one or more activation associated molecules selected from the group consisting of IL-2, IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, for evaluating formation of germinal center-like (GC) clusters of cells for determining whether said human's cells are responding to said test substance. In one embodiment, said B cell activation medium contains only two activation associated molecules (along with the test substance), namely IL-21 and soluble CD40L molecules (i.e. it does not contain IL-2). In one embodiment, said soluble CD40L is provided by CD40L expressing feeder cells.

In one embodiment, the present invention provides a method for evaluating formation of germinal center-like (GC) clusters of cells, comprising, a) providing; i) a microfluidic device comprising a space located in between an inlet and an outlet; ii) a human cell population comprising B cells, said cells suspended in a hydrogel precursor; and iii) a B cell activation medium, comprising a test substance and one or more activation associated molecules selected from the group consisting of IL-21 and soluble CD40L molecules; b) introducing said hydrogel precursor into said space; c) treating said hydrogel precursor under conditions so as to at least partially solidify said hydrogel; and d) flowing said B cell activation medium under conditions such that said B cells are exposed to said medium, for evaluating formation of germinal center-like (GC) clusters of cells for determining whether said human's cells are responding to said test substance. In one embodiment, said B cell activation medium contains both activation associated molecules (along with the test substance), namely IL-21 and soluble CD40L molecules (and it does not contain IL-2). In one embodiment, said soluble CD40L is provided by CD40L expressing feeder cells. In one embodiment, said evaluating comprises density, size and morphological observations of cell clusters. In one embodiment, said evaluating indicates an antigenic response to said test substance. In one embodiment, said evaluating indicates a lack of response to said test substance. In one embodiment, said density is evaluated from light to dark, wherein darker clusters indicate a stronger antigenic response. In one embodiment, said size ranges from small to large, wherein larger clusters indicate a stronger antigenic response. In one embodiment, said clusters become larger in diameter over time indicating a stronger antigenic response. In one embodiment, said clusters are observed to become darker over time.

Use of an AB-Lymph Node Chip is not limited to WBCs obtained from humans. Indeed, WBCs from any mammalian source may be used where the compositions and methods described herein induce measurable antibody production. Moreover, designs of microfluidic devices described herein are contemplated for use with other mammalian systems where compositions and methods are known for initiation antibody production in plates or bioreactors. One non-limiting example is where WBCs are hybridoma cells and AB-Lymph Node Chips produce high levels of monoclonal antibodies for noncommercial and commercial use.

In one embodiment, the present invention contemplates both a device and the use of a device comprising two membranes (a dual membrane microfluidic device). A variety of dual membrane designs can be used, including those shown in U.S. Pat. No. 8,647,861, hereby incorporated by reference. In one embodiment, the dual membrane device comprises a layer between the two membranes, including (but not limited to) a gel layer comprising one or more channels connected to one or more gel ports (see FIG. 61). A lid can be used as a gel port block, e.g. after material such as gel precursor has been introduced via the gel ports.

In one embodiment, a gel is placed between the two membranes. In one embodiment, the microfluidic device has one or more ports (e.g. two ports) that allow for the introduction of a gel or (more preferred) a gel precursor. In one embodiment, the present invention further contemplates blocking said one or more ports after introducing said gel or gel precursor. In one embodiment, the ports are blocked with a lid that serves as a port block. In one embodiment, the gel port block also serves to inhibit or prevent deformation of the device. In one embodiment, the microfluidic device has other (unblocked) ports that align with culture module as described in U.S. Pat. No. 10,273,441, hereby incorporated by reference. In one embodiment, the gel port block allows the microfluidic device to be compatible with the interface of such a culture module. In one embodiment, the gel port block engages the upper portion of the device by sliding, thereby covering the gel ports. In one embodiment, the gel port block snaps into place, covering the gel ports.

In one embodiment, the gel is attached to a surface of a device, e.g. the surface and/or walls of a channel, cavity, chamber or the like. In one embodiment, an agent is used to better secure the gel to the surface. In one embodiment, the agent is a bifunctional crosslinker, such as ER1. In one embodiment, the agent is polydopamine. In one embodiment, the device is a microfluidic device or chip. In one embodiment, the device is a transwell. In one embodiment, said gel comprises cells (such as the B cells described earlier). In one embodiment, at least a portion of said gel is exposed to culture media. In one embodiment, said culture media is a flow of culture media. In one embodiment, there is no flow and the culture media is static. In one embodiment, there is flow in channel lacking a gel and no flow in the channel comprising the gel.

Thus, in one embodiment, the present invention contemplates a device (including but not limited to a microfluidic device) comprising a surface, said surface comprising a gel attached thereto via an agent. In one embodiment, the agent is a bifunctional crosslinker. In one embodiment, the agent is polydopamine. In one embodiment, said gel comprises cells (such as the B cells described earlier). In one embodiment, at least a portion of said gel is exposed to culture media. In one embodiment, said culture media is a flow of culture media. In one embodiment, there is no flow and the culture media is static. In one embodiment, there is flow in channel lacking a gel and no flow in the channel comprising the gel.

In one embodiment, the present invention contemplates a method wherein a) cells (such as B cells) are suspended in a hydrogel precursor; b) the gel precursor (e.g. hydrogel precursor) is introduced through gel ports into a space (e.g. a cavity, channel, chamber, etc.) on the device; c) the gel precursor (e.g. hydrogel precursor) is treated under conditions so as to at least partially solidify said gel (e.g. hydrogel); and d) the gel ports are blocked. In one embodiment, the gel ports are blocked by a lid or gel port block that engages the top of microfluidic device having other (unblocked) ports. In one embodiment, the device is a microfluidic device. In one embodiment, the method further comprises linking the microfluidic device to a culture module which introduces culture fluid into one or more unblocked ports. In one embodiment, said microfluidic device comprises first and second membranes with the gel or hydrogel positioned between said membranes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced figures. It is intended that the embodiments and figures disclosed herein are to be considered illustrative rather than restrictive.

FIG. 1A shows an exemplary illustration of a human Lymph Node. Willard-Mack. 2006.

FIG. 1B shows an exemplary illustration of human B cell differentiation while proliferating, starting from a precursor cell that provides both a CD3− (B cell lineage) and a CD3+ (T cell lineage).

FIG. 1C shows an exemplary illustration of a human Lymph Node Follicle illustrating light zones and dark follicular zones comprising activated B cell clusters. De Silva and Klein. 2015.

FIG. 2A shows exemplary bright field microscopic images of human B cell cultures during incubation in 2D cultures (e.g. plates) using one embodiment of a 3 step method over 10 days as described herein. Day 3 cells after initiating activation on Day 0 showing some clustering, Day 4 cells in differentiation medium showing larger and denser clusters than on Day 3, Day 6 changing to maintenance medium showing large-dark/dense clusters, and Day 10 showing large-dark/dense clusters. Starting cells are purified CD3-CD19+CD27+ human B cells.

FIG. 2B shows exemplary flow cytometry data of human B cells harvested in 2D cultures (e.g. plates) using one embodiment of a 3 step method over 10 days as described herein. Hydrogels were dissolved for releasing cells having intact cell surface biomarkers for use in antibody tagging for flow cytometry. Day 0 results profiled gated live cells for a CD19+CD27+ purified B cell population in turn showing the majority of cells are CD38-CD20+ Memory B cells and CD38^{lo}. Day 3 cells after initiating activation on Day 0 showing some clustering, Day 4 cells in differentiation medium showing larger and denser clusters than on Day 3, Day 6 changing to maintenance medium showing some large-dark/dense clusters, and Day 10 showing some large-dark/dense clusters. Starting cells are purified CD3-CD19+CD27+ human B cells.

FIG. 3 shows an exemplary schematic illustration of one embodiment of a microfluidic device shown as a S1 (tall channel) organ-chip comprising upper (light) and lower (dark) cell culture microchannels with a microfabricated porous elastic membrane sandwiched in-between. In some embodiments a microdevice may also be equipped with two full-height, hollow microchambers alongside of the cell culture channels. An exemplary organ chip has: 1. Epithelial Channel; 2. Epithelial Cells, e.g. primary cells, cell lines, Caco2, primary intestinal cells, cancer cells, etc.; 3. Optional Vacuum Channel; 4. Membrane, optional stretch; 5. Endothelial Cells e.g., human Intestinal HIMEC or iHIMEC, etc.; and 6. a Vascular Channel. See, WO 2010/009307A2, herein incorporated by reference in its entirety.

FIG. 4 shows an exemplary schematic illustration of one embodiment of a S1 microfluidic device (upper) with an enlarged schematic of a membrane 208, upper side 208A, lower side 208B, separating two channels. Unlike other organ microfluidic devices, for lymph node-chips producing antibodies, endothelial cells were not typically seeded into either of the channels. In fact, the presence of endothelial cells on-chip during variable testing interfered with antibody production. For lymph node-chips producing antibodies, hydrogels were flowed through the lower channel for filling space within these channels. For testing hydrogel integrity, there was no direct flow in the lower channel while a constant flow was provided in the upper channel. Grey solid depicts medium in the upper channel under flow while grey speckled depicts the solidified hydrogel filling the lower channel under the membrane that is not under direct fluid flow through the lower channel.

FIG. 5 shows an exemplary illustration of one embodiment of a S1 (tall channel) chip where the lower channel was coated with comparative formulations of hydrogels stained for gel proteins. 1:1 Matrige®:bovine collagen I (Fibricol®); 2 mg/mL Rat Tail Collagen I; 2 mg/mL bovine Collagen I (Fibricol®). Lower image shows a stained hydrogel within a channel.

FIG. 6 shows an exemplary isolated peripheral white blood cell population immunostained for CD45 (upper white cells) demonstrating a range of sizes and shapes. Scale bar is 100 μm. In some embodiments, the entire CD45+ population resulting from isolation produces is used as described herein. In some embodiments, CD19+CD27 white blood cells are purified into a population for use as described herein. An exemplary illustration is shown (lower) of one embodiment of an S1 organ-chip configuration where the bottom channel is filled with a hydrogel—white blood cell mixture (e.g. PBMCs embedded in a hydrogel as described herein) for producing antibodies. During co-culture, the top channel, under media flow, grey, is separated by a membrane from the Bottom Channel not undergoing direct flow during incubation in culture medium.

FIG. 7 shows exemplary flow cytometry data of B cells immunostained for activation and maturation markers demonstrating B Cell Differentiation in 3D cultures (e.g. one embodiment of a Lymph Node-Chip). Live CD19+CD3− cells derived from a CD19+CD27+ WBC population profiling (outlined in thick black boxes) CD38−CD20+ memory B cells, activated CD19+CD27+ cells, CD38+CD20− plasmablasts and CD38+CD138+ plasma cells. GC B cells are CD38+CD20+.

FIG. 8 shows exemplary bright field microscopic images of PBMCs in a microfluidic chip undergoing one embodiment of a differentiation protocol. Day 0—upper image. D10-lower images, each panel showing images from different donors. B=bottom channel. T=top channel. The lower gel is frill of cells including large clusters of cells indicative of germinal centers (GCs) with the most individual cells in the middle and large clusters towards the outlets. The middle of the bottom channel is full of cells, appearing black in these images. Both the top channel outlet and bottom channel outlet were full with cells. Cells that migrated into top channels formed large cell clusters indicative of germinal centers (GCs).

FIG. 9 shows exemplary flow cytometry data of B cells derived from CD19+CD27+ B cells immunostained for activation and maturation markers demonstrating populations of B Cells between top and bottom channels at Days 6 and 10.

FIG. 10 shows exemplary flow cytometry data of B cells derived from PBMCs immunostained for activation and maturation markers demonstrating populations of B Cells between top and bottom channels at Days 0 and 10.

FIG. 11 shows exemplary IgM and IgG measured from top channel effluent. IgM was detected up to 40,000 ng/ml. IgG was detected in concentrations greater than 100,000 ng/ml up to 380,000 ng/ml. Each line represents an individual donor/chip. A second graph of IgM is provided using the same scale as the IgG graph for a direct comparison of amounts. Effluent samples (200 ul) were collected every 24 hours then accumulated-combined, e.g. Day 10 amounts were measured in samples collected on days 6, 7, 8, 9 and 10. A three step method using B cell activation, differentiation and maintenance media was used as described herein.

FIG. 12 shows exemplary amounts of IgG measured in upper channel effluent after non-specific stimulation compared between fractionated (CD19+CD27+ purified from PBMCs), lower dark symbols and lines, and unfractionated (total) PBMCs, upper light symbols and lines, in one embodiment of a Lymph Node-Chip. A three step method using B cell activation, differentiation and maintenance media was used as described herein.

FIG. 13 shows an exemplary comparison of IgG measured in upper channel effluent after antigen-specific stimulation, using Tetanus Toxoid (TT), of unfractionated (total) PBMCs. A three step method using B cell activation, differentiation and maintenance media was used as described herein, however with the lack of IL-2 and goat anti-human Fab2 fragments with or without TT in the stimulation media.

FIG. 14 shows exemplary flow cytometry data of a replicate Lymph Node chip seeded with total PBMCs immunostained for activation and maturation markers demonstrating populations of live gated B Cells at Day 4 combined top and bottom channel, Day 7 and Day 10 top vs. bottom channels. Nonspecific stimulation using a 2 step procedure as described herein.

FIG. 15 shows exemplary flow cytometry staining and a cell gating strategy for evaluating tonsil white blood cell types seeded in plates and microfluidic chips for undergoing a 3 step culture as described herein. 1) CD19+CD3− cells were gated into CD19+CD3−CD27− cells and CD19+CD3−CD27+ cells. 2) CD19+CD3−CD27− cells were gated into CD38+CD20− plasma cells, CD38+CD20+ germinal center cells and CD38−CD20− memory B cells. 3) CD19+CD3−CD27+ cells were gated into CD38+CD20− plasma cells, CD38+CD20+ germinal center cells and CD38−CD20− memory B cells

FIG. 16 shows exemplary flow cytometry staining of CD19+CD3−CD27+ tonsil cells gated into plasma cells, germinal center cells and memory cells comparing CD138, HLA-DR, CD30 and CD32b cell surface expression levels.

FIG. 17 shows an exemplary workflow for compositions and methods using soluble recombinant human CD40L.

FIG. 18 shows exemplary flow cytometry staining of CD19+CD27+ B cells, isolated from PBMCs, stimulated in the presence of a bacterial CpG DNA repeat segment (lower panels) and histidine tagged soluble recombinant human CD40L in a stimulation medium, using a 3 step, 3 media differentiation method over 10 days, in 6 well plates. Upper panels show duplicate cultures without CpG DNA, right bright field image shows single cells while the lower bright field image shows numerous small cellular clusters in the presence of CpG DNA antigen.

FIG. 19 shows exemplary flow cytometry staining of cells shown in the previous figure without CpG DNA gated into plasma cells, germinal center cells and memory cells for comparing CD138, HLA-DR, CD30 and CD32b cell surface expression levels. Germinal center cells are shown in the middle column.

FIG. 20 shows exemplary flow cytometry staining of cells with CpG DNA gated into plasma cells, germinal center cells and memory cells for comparing CD138, HLA-DR, CD30 and CD32b cell surface expression levels. Germinal center cells are shown in the middle column.

FIG. 21 shows exemplary flow cytometry staining of cells without CpG DNA left panels and with CpG DNA right panels of Day 4 and 7, gated into plasma cells, germinal center cells and memory cells.

FIG. 22 shows exemplary flow cytometry staining of B cells over time in 96 well plates using soluble recombinant human CD40L, from CD19+ B cells showing a percentage of the memory cells producing germinal center cells then plasma cells.

FIG. 23 shows exemplary IgG production over time from CD19+CD27+ B cells using soluble recombinant human CD40L in 96 well plates.

FIG. 24 shows exemplary flow cytometry staining of B cells on Day 4 in 96 well plates from CD19+CD27+ B cells soluble recombinant human CD40L, from CD19+ B cells showing a percentage of the memory cells producing germinal center cells then plasma cells at Day 0 and Day 4, with CpG DNA.

FIG. 25 shows exemplary flow cytometry staining of B cells on Day 4 in 96 well plates from CD19+CD27+ B cells using the method including recombinant CD40L molecules from CD19+ B cells showing a percentage of the memory cells producing germinal center cells then plasma cells at Day 0 and Day 4, with CpG DNA.

FIG. 26 shows exemplary flow cytometry staining of B cells on Day 4 in 6 well plants and 96 well plates from CD19+CD27+ B cells using the method including recombinant CD40L molecules, from CD19+ B cells showing a percentage of the memory cells producing germinal center cells then plasma cells at Day 0 and Day 4, with and without a media change.

FIG. 27 shows an exemplary flow cytometry summary of dot plots demonstrating staining of B cells over time comparing 6 well plates and 96 well plates from seeded B cells using the method including recombinant CD40L molecules, for CD19+ B cells showing a percentage of the memory cells, germinal center cells and plasma cells.

FIG. 28 shows an exemplary workflow for compositions and methods modified using recombinant CD40L.

FIG. 29 shows exemplary flow cytometry staining of CD19+CD27+ B cells, isolated from PBMCs, stimulated in the presence of a bacterial CpG DNA repeat segment (lower panels) and histidine tagged soluble recombinant human CD40L in a stimulation medium, using a 3 step, 3 media differentiation method over 10 days, in microfluidic chips.

FIG. 30 shows exemplary IgG and IgM production over time from CD19+CD27+ B cells using soluble recombinant human CD40L in microfluidic chips. IgG amounts are greater than previously shown from plate experiments.

FIG. 31 shows an exemplary Workflow for compositions and methods using gamma irradiated CD40L feeder cells.

FIG. 32 shows exemplary flow cytometry staining of CD19+CD27+ B cells, isolated from PBMCs, stimulated with or without the presence of CD40L expressing feeder cells in a stimulation medium, using a 3 step, 3 media differentiation method over 10 days, in 6 well plates. Upper panels show duplicate cultures without CD40L feeder cells, right bright field image shows single cells while the lower bright field image shows numerous slight and dark large cellular clusters.

FIG. 33 shows exemplary flow cytometry staining of CD19+CD27+ B cells, isolated from PBMCs, stimulated with or without the presence of gamma irradiated CD40L expressing 293T feeder cells in a stimulation medium of the present inventions but lacking soluble CD40L, using a 3 step, 3 media differentiation method over 10 days, in 6 well plates.

FIG. 34 shows exemplary day 10 flow cytometry staining of CD19+CD27+ B cells, isolated from PBMCs, stimulated with the presence of gamma irradiated CD40L expressing 293T feeder cells in a stimulation medium of the present inventions but lacking soluble CD40L, using a 3 step, 3 media differentiation method over 10 days, in 6 well plates. Lower panels, gamma irradiated CD40L expressing 293T feeder cells were added in the flow media from Day 7-Day 10.

FIG. 35 shows exemplary clustering of cells on Days 3-at least Day 6 compared to day 10 in 6 well plates using the method described in the previous figure including gamma irradiated CD40L expressing 293T feeder cells.

FIG. 36 shows exemplary IgG production over time from CD19+CD27+ B cells using recombinant CD40L in microfluidic chips compared to IgG amounts using CD40L feeder cells.

FIG. 37 shows exemplary bright field images on Day 10, using recombinant CD40L in plates.

FIG. 38 shows exemplary flow cytometry and IgG production on Day 10, using recombinant CD40L in plates.

FIG. 39 shows an exemplary workflow for compositions and methods using gamma irradiated CD40L feeder cells.

FIG. 40 shows exemplary clustering of cells on Day 3 and Day 10 comparing clustering in 6 well plates using the modified method including gamma irradiated CD40L expressing 293T feeder cells of cells in plates coated with no gel, Matrigel and two different amounts of collagen.

FIG. 41 shows exemplary clustering of cells on Day 3 and Day 10 comparing clustering in 6 well plates using the modified method including gamma irradiated CD40L expressing 293T feeder cells of cells in plates coated with no gel, Matrigel and two different amounts of collagen.

FIG. 42 shows exemplary flow cytometry staining of live-dead cells using methods including gamma irradiated CD40L expressing 293T feeder cells of cells in plates coated with no gel, Matrigel and two different amounts of collagen. Exemplary comparisons of IgG production.

FIG. 43 shows exemplary flow cytometry staining of live-dead cells using methods including gamma irradiated CD40L expressing 293T feeder cells of cells in plates coated with no gel, Matrigel and two different amounts of collagen. Exemplary comparisons of activated cells, germinal center cells and plasma cells.

FIG. 44 shows one exemplary embodiment Workflow methods including gamma irradiated CD40L expressing cells for differentiation along with exemplary flow cytometry staining of CD19+CD27+ cells, stimulated with or without the presence of CD40L expressing feeder cells in a stimulation medium including CD40L, IL-2, IL10, for 3 days followed by 4 days in either IL-2, IL-10, CD40L with or without CD40L feeder cells, and lower dot plot with CD40L and antibody blocking CD40L without CD40L feeder cells.

FIG. 45 shows exemplary flow cytometry staining of CD19+CD27+ cells, as in the previous figure.

FIG. 46 shows exemplary clustering of cells on Days 3, 7 and 10 comparing clustering in 6 well plates using the modified method including gamma irradiated CD40L expressing 293T feeder cells of cells in plates coated with no gel, Matrigel and Fibricol. Darker clusters develop using Fibricol. Lower panels show exemplary IgG production over time from CD19+CD27+ B cells using the method with or without CD40L feeder cells and with or without blocking CD40L antibody.

FIG. 47 shows exemplary flow cytometric analysis of cell populations using the method including CD40L feeder cells in the presence of hydrogels of Matrigel, rat tail collagen and bovine collagen, Fibricol, in 6 well plates comparing donors and over time.

FIG. 48 shows exemplary IgG production over time from CD19+CD27+ B cells using CD40L feeder cells in the presence of hydrogels of Matrigel, rat tail collagen and bovine collagen, Fibricol, in 6 well plates. IgG production is similar throughout the different gel composition. The main factor appears to be donor variability using this method.

FIG. 49 shows exemplary biomarker panel for monocyte related biomarkers.

FIG. 50 shows exemplary flow cytometry staining for Confirmation of Flow Cytometry Panel by FMO (Fluorescence Minus One Control, or FMO control) referring to a type of control used to properly interpret flow cytometry data. It is used to identify and gate cells in the context of data spread due to the multiple fluorochromes in a given panel.

FIG. 51 shows an illustration of an exemplary lineage of monocytes and CD4 cells by selected biomarker expression.

FIG. 52 shows exemplary flow cytometry staining of tonsil cells using monocyte and CD4 cell biomarkers from the previous figures.

FIG. 53 shows exemplary flow cytometry gating strategy for monocyte and CD4 cells from cultures.

FIG. 54 shows exemplary flow cytometry staining of PBMCs and tonsil cells obtained from multiple donors using CD40L feeder cells.

FIG. 55 shows exemplary flow cytometry staining on Day 10 comparing embodiments of microfluidic chips seeded with WBCs in hydrogels from either PBMC D10 (Gated on Live Cells) or Tonsil MNC D10 (Gated on Live Cells) compared to the same methods but using CD19+CD27+ cells in Wells (Gated on Live Cells) using a 3 step procedure over 11 days including T follicular helper cells demonstrating more: cells, live cells, CD19+ cells and CD27+ cells in tonsil white blood cell populations.

FIG. 56 shows an exemplary work flow using a 3 step procedure over 11 days including T follicular helper cells with several exemplary embodiments for substance testing shown.

FIG. 57 shows exemplary flow cytometry staining of CD4, CD45RA, showing a purified CD4+CD45RA+ subset gated for showing percentages of CD4+CXCR5+ Follicular T cells. −CL075−SAC; +CL075−SAC; +CL075+SAC.

FIG. 58 shows exemplary bright field micrographs comparing clustering of monocytes 24 Hours Post Activation in the presence of SAC in the presence of different concentrations of CL075, a thiazoloquinolone derivative that stimulates TLR8. Chart shows exemplary IL-12p70 secretion over low to high concentrations of CL075. 0 ug/mL CL075 1 ug/mL CL075 10 ug/mL CL075.

FIG. 59 shows exemplary bright field micrographs comparing clustering of monocytes 24 Hours Post Activation in the presence of SAC and GG in the presence of different concentrations of CL075, a thiazoloquinolone derivative that stimulates TLR8. MOI 1 and MOI 10 both yield the same amount of IL-12.

FIG. 60 shows exemplary IL-12/L-23p40 secretion over low to high concentrations of CL075 comparing 2 modified methods.

FIG. 61 shows one embodiment of a dual membrane device with a gel port block (for blocking the gel ports after use) and gel layer comprising a channel. The gel ports are aligned with the channel of the gel layer. Other ports (which are not blocked) permit engagement with a culture module for the introduction of culture media for perfusion of cells. The gel ports can be lower in height (as compared to the other ports) so as to allow the gel port block to cover the gel ports.

### DEFINITIONS

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

As used herein, the term “biopsy” refers to a sample of the tissue that is removed from a body.

### DESCRIPTION OF INVENTION

The present invention relates to fluidic systems for producing IgG antibodies from co-cultures of white blood cells. In some embodiments, a microfluidic device containing co-cultures of autologous whole peripheral white blood cell populations, including B cells, are used for providing antigen specific IgG antibody production from differentiating B cells (plasma cells). More specifically, high levels of IgM and IgG classes of antibodies are harvested from fluids flowing through the device. In some embodiments, IgG is produced during activation in the presence of antigen, including but not limited to therapeutic immunogenic compounds, e.g. engineered antibodies, vaccines, etc. In some embodiments, such co-cultures are further exposed to drug compounds e.g. for preclinical safety testing and individualized personal drug responses. In some embodiments, such antibody producing microfluidic devices are contemplated for use in companion diagnostic and complementary assays.

Currently there is no reliable in vitro assay for mimicking in vivo human IgG (immunoglobulin G) antibody production induced by antigenic stimulation, especially in relation to preclinical safety testing and for accurately predicting endogenous antibody related adverse effects induced by pharmaceutical treatments. As discussed herein, animal models, including the use of humanized/engineered mice and primates, typically either under estimate or over estimate safety concerns during preclinical in vivo tests. Such inaccurate results cause millions of wasted dollars through missing adverse preclinical responses in humans; loss of a potentially effective human treatments along with indefinitely delaying the development of viable pharmaceuticals needed for treating human diseases.

Therefore an in vitro replacement of in vivo animal preclinical testing is needed. Further, for enhanced safety to human volunteers during initial clinical tests, where unexpected adverse responses are first revealed, it would be useful to have a diagnostic assay for accurately predicting adverse IgG antibody responses. In contrast, it would be reassuring to patients that a particular pharmaceutical was cleared for adverse reactions under more accurate in vitro testing in a more human-like environment. Such in vitro testing such as described herein in the present invention, prior to FDA approval and commercial use in a general population of people which includes diverse populations of genetic backgrounds with a variety of disease backgrounds, would increase the safety of pharmaceutical use in humans and at least allow physicians to provide more accurate assessments of potential adverse reactions to their patient populations.

Moreover, current in vivo tests for vaccine safety and antibody responses to the vaccine antigen are often conducted using animals including primates. Such vaccine testing suffers the same problems as in vivo preclinical safety testing of pharmaceutical treatments.

Further more, drugs and vaccines intended for administration to a large number of people in a general population that may not show classical antigenic reactions during preclinical testing, do react as antigen for stimulating a minor subpopulation of B cells resulting in an unexpected clinically significant endogenous antibody response inducing unwanted adverse reactions that may cause death.

As described herein, the present invention provides compositions and methods for testing immunogenicity of an agent (e.g. antigen, pharmaceutical, biopharmaceutical, etc.) resulting in production of IgG. IgG antibody production is not limited to any one type of antigenic stimulation. Indeed, IgG antibody production may result from one or more of, and is not limited to direct pharmaceutical (or antigen) stimulation and/or cross-reactive stimulation of immune cells that are not limited to energized B cells, memory B cells, stimulation of naive B cells which induces maturation, i.e. isotope specific immunoglobulin class switching, etc.

### I. In Vivo Immunological B Cell Production of Endogenous Antibodies.

Production of endogenous antibodies in vivo is a complicated part of the humeral immune system comprising numerous interacting cell types, often not merely limited to interactions of immune cells themselves, as much as researchers wish it was so limited. Therefore, descriptions of antibody production, both in vivo and in vitro described herein, are presented merely for orientation and not intended as an exhaustive explanation for in vivo or in vitro processes, related to types of B cell populations, and physiology and biology associated with proliferation, activation, differentiation, terminal differentiation, memory, reactivation, apoptosis, etc., associated with anti-self reactions, idiotypic reactions, antigen-specific reactions, affinity maturation of B-cell repertoires/B-cell receptor (BCR) during reactions, cross activation-reactions, drug reactions, etc., often resulting in a unanticipated antibody production and/or unexpected antibody targeting of molecules.

Thus, in part, the following general description of antibody production in response to B cell stimulation is provided merely for context of in vitro environments created for in vitro antibody production mimicking in vivo antibody production as described herein. B cells refer to any type of B cell within the B (lymphocyte) cell lineage, including but not limited to in order of development or terminology): pre-pro-B− cells (CD19⁺CD10⁻CD34⁺), e.g. having D-J_{H} gene rearrangements, pro-B cells (CD19⁺CD10⁺CD34⁺), e.g., having rearrangements of V_{H}-to-DJ_{H} genes, where any one of these cells typically develop in mammalian bone marrow or lymphoid tissue and may circulate through the peripheral blood or lymph. After certain steps, mature B cells as naïve B cells or energized B cells may further differentiate into stages of i) activated-stimulated-transitional cells for undergoing either further differentiation during proliferation into plasmablasts/plasma cells, or spin offs of daughter cells into ii) memory B-lymphocytes, under certain conditions differentiation into iii) plasmablasts, iv) plasma cells (in most cases considered terminally differentiated cells). In most cases, Naive B cells may be identified as having an unmutated IgV region sequence while simultaneously co-expressing cell surface IgM and IgD. In contrast, for normal healthy people memory B-cells (≈20-30% of all PB B-cells) display mutations within their IgV regions, and about half have switched sIgH (≈23%±10% and ≈21%±9% of adult PB memory B-cells express s(surface)IgG and sIgA, respectively). The other half memory B-cells still coexpress sIgM and sIgD (≈52%+15% of memory B-cells), or potentially mainly sIgM (soluble IgM).

During differentiation of naïve to memory B-cells, B-lymphocytes acquire a higher antigen binding affinity of BCRs, at the same time they change the expression patterns of multiple surface receptors and intracellular factors that increase their responsiveness. Accordingly, memory B-cells show a higher in vitro response (vs. naïve B-cells) against different stimuli that mimic Ag-recognition (i.e., anti-BCR antibodies) and/or interaction with T-helper cells (i.e., CD40L), with or without the support of cytokines and/or TLR ligands. Once exposed to these factors, memory B-cells rapidly enter the cell-cycle, they undergo more rounds of division and a larger proportion of them become Ab-secreting cells (plasmablasts and PC).

A lymphatic system is found in the majority of mammals, inducing specialized discrete organs surrounded by capsules that can be dissected out, e.g. thymus, spleen, lymph nodes, including draining lymph nodes, tissue specific lymph nodes, tonsils, appendix, spleen, etc. connected by relatively slow moving fluids through a circulating lymphatic duct system and either have direct or indirect contact with a faster moving arterial-venous blood circulatory system. Some lymphoid tissue is more diffuse, e.g. as lymphoid masses, such Peyer's patches (aggregated lymphoid nodules), lymphoid follicles, both organized and unorganized, bone marrow and ectopic lymphoid tissue sometimes found associated with diseases. Diffuse lymphoid tissue is located next to or within diffusion/migration distance of lymphatic and blood circulatory systems. In vivo, both white blood cells and nonwhite blood cells in addition to molecules and proteins, e.g. cytokines, chemokines, antigens, dynamically migrate from organ to organ or flow around a body through at least one or both of these circulatory systems.

In general, locations in vivo where white blood cells are exposed to antigen, under certain conditions, leads to inter- and intra-cellular interactions sometimes resulting in low to high levels of antibody production, e.g. IgM, IgG, IgA, IgE and IgD. Including maturation to IgG₁, IgG₂, IgG₃, IgA₁, and IgA₂.

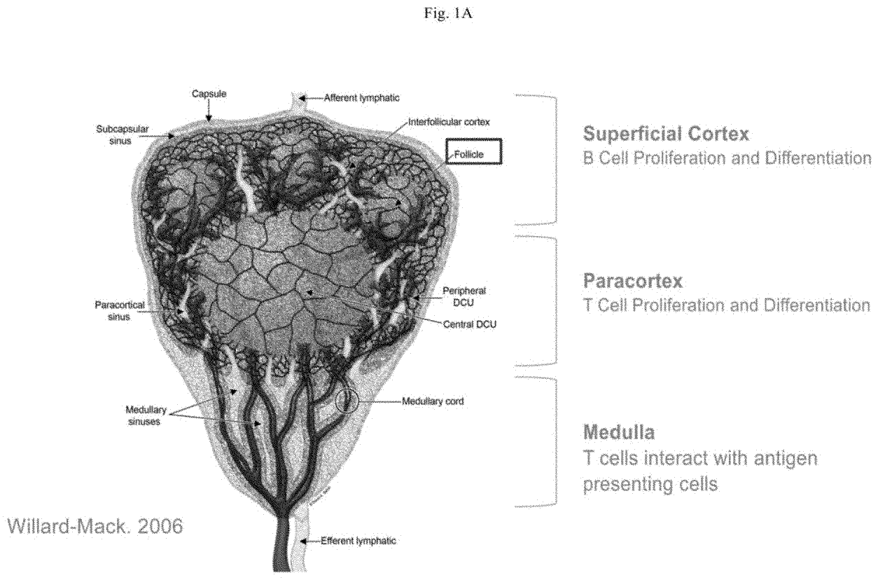

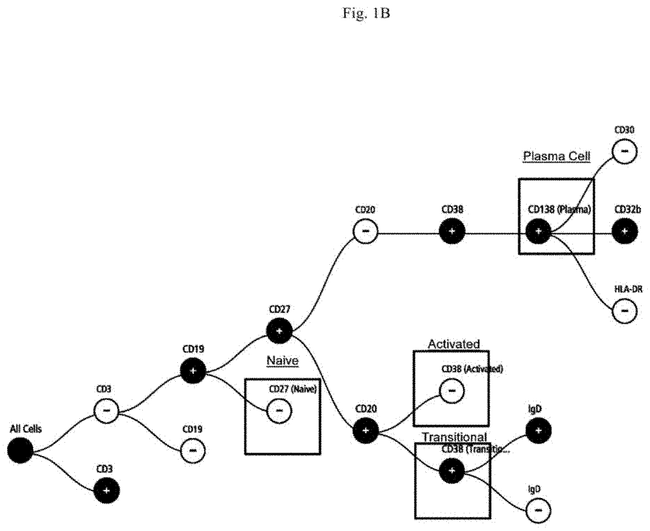

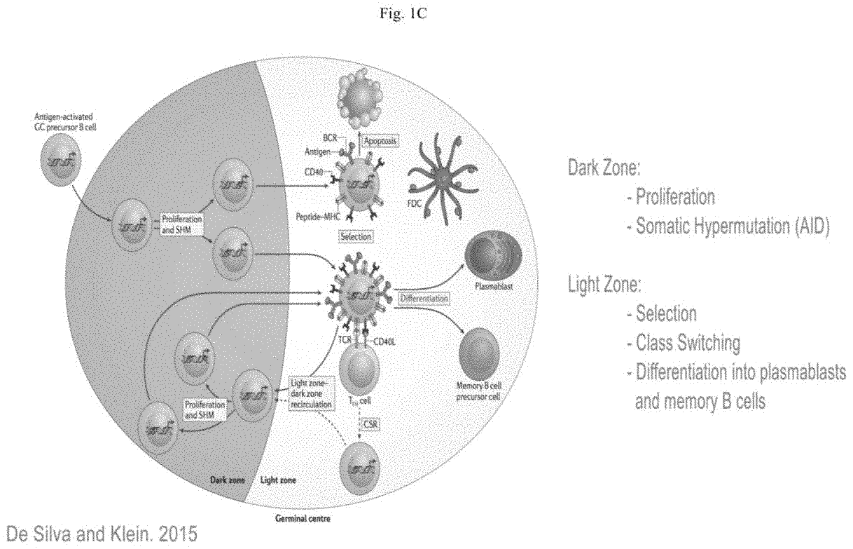

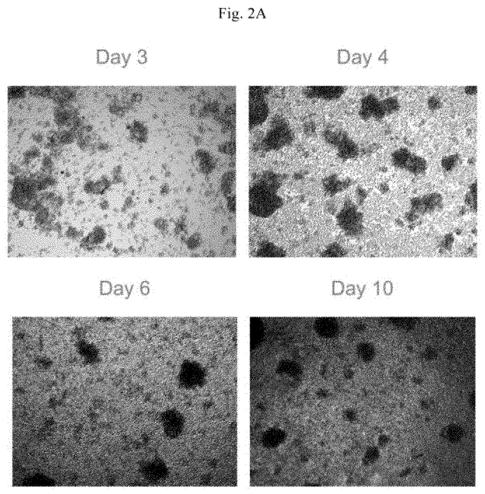

Despite numerous attempts to provide an in vitro environment to replicate in vivo high levels of induced IgG production, often termed “lymph node” chips or systems in response antigen has not been publicly demonstrated. Further, high levels of antibodies induced in vitro were not shown to provide a mixture comprising a range of specific, i.e. producing antibodies from low to high affinity to (as in “binding to”) antigenic epitopes to antigen over time. FIG. 1A shows an exemplary illustration of a human Lymph Node. Willard-Mack. 2006.

Using an in vitro lymph node model for comparison as described herein, is merely one example of an in vivo immunological based process of resulting in antibody production. In vivo, healthy lymph node histology shows a superficial cortex area where mainly B cells (CD3−CD19+CD45+) proliferate and differentiate; a paracortex where mainly T cells (CD3+CD19−CD45+) proliferate and differentiate; medullary areas, including medullary cords where T cells interact with antigen presenting cells. Of particular interest are the follicular areas where resident B cells interact with antigen within a certain range of avidity and T resident cells, mainly CD4+ T helper cells (CD3+CD4+CD19−CD45+), undergo activation which under certain environmental conditions results in the foil cation of germinal centers within that follicle so long as that follicle contains B cells having that particular antigenic recognition. As the B cells undergo differentiation during cell division, resulting plasmablasts and plasma cells migrate into areas near outgoing flowing circulatory systems for secreting antibodies into the lymphatic and blood steams. FIG. 1B shows an exemplary illustration of human B cell differentiation while proliferating, starting from a precursor cell that provides both a CD3− (B cell lineage) and a CD3+ (T cell lineage).

Jumping into one scenario of in vivo antibody production within one individual germinal center, when a presumptive antigen is recognized (binds to) an antigenic receptor expressed on the surface of, lets say a naive B cell (first time encountering that particular antigen or other types of stimulation), within a particular range of avidity that is recognized by that cell as triggering an activation/proliferation response and under certain supportive environmental conditions then both antibody-secreting cells (ASCs) secreting soluble antibody and memory cells are produced. In the case of ASCs, these cells are plasmablasts which may terminally differentiate into plasma cells secreting high levels of antibodies. As for the memory B cells, they may survive within a body for many years, in part to allow the immune system to have on tap cells that previously underwent activation/proliferation which then respond faster to provide antibodies upon exposure to antigen after a first wave of antigen exposure, i.e. after plasma cells die off through natural apoptosis. FIG. 1C shows an exemplary illustration of a human Lymph Node Follicle illustrating light zones and dark follicular zones comprising activated B cell clusters. De Silva and Klein. 2015.

Thus, histologically and after strenuous-exhaustive research efforts, dark and light zones are observed with associated physiological functions. In general, dark zones represent areas of cellular proliferation where in between cell divisions, DNA of these proliferating cells undergoes somatic hypermutation (AID) resulting in genomic alterations in daughter cells. Light zones in general represent areas where stimulated-activating B cells undergo selection, isotype switching, subclass switching while differentiating into plasmablasts and memory B cells.

### II. In Vitro Immunological B Cell Production of Endogenous Antibodies.

Each individual, including twins siblings, relatives, etc., even cloned animals, have unique populations of B cells that are different from each other. That said, in general, healthy individuals who are closely related likely have similar ranges of antigen responsive B cells while larger and less related larger healthy populations loose or gain antigenic reactivity in relation to an individual. In contrast, when observing individuals at risk for, or having a disease, these individuals may have starkly different types of antigenic reactivity compared to healthy individuals/populations. These ranges of B cell reactivity become more diverse when individuals and populations are exposed to new antigens, including immunogenic therapeutics, drug treatments, even exposure to cosmetics and food products, etc., over time.

Therefore, there is a need for identifying ranges of reactivity to compounds that develop from compounds administered to individuals and populations. Especially when such B cell reactivity results in adverse reactions to a clinical treatment intended to improve health, not induce worse consequences to the patient. See also, the section on Companion Diagnostics, below.

Therefore, strenuous research was and is currently being done in order to mimic in vivo endogenous B cell responses within an in vitro device. As described herein, the majority of in vitro devices for simulating endogenous B cell responses rely upon tissue culture plates, transwells and microfluidic devices and in at least one example, a bioreactor. However, none of these in vitro devices appear to provide microenvironments for B cell activation, proliferation, and differentiation which induce high levels of plasma cells producing at least copious amounts of IgG secreted into surrounding fluid. And most especially, not within 4-10 days of initiation of whole white blood peripheral cell culture.

As described herein, plate cultures were initially used for testing medium formulations, for comparing different compositions of white blood cell populations, and for determining time frames of antibody production in response to test variable comparisons. Thus, in some embodiments, methods and compositions described herein are used in plate and multi-well plate cultures. In some embodiments, methods and compositions described herein are used in transwell plates. In some embodiments, methods and compositions tested in plates and are then replicated in microfluidic devices. In some embodiments, methods and compositions are developed on-chip using microfluidic devices described and referred to herein. In some embodiments, methods and compositions developed on-chip are unique for use in microfluidic devices described and referred to herein.

A. B Cell Differentiation in 2D Cultures (e.g. Plates).

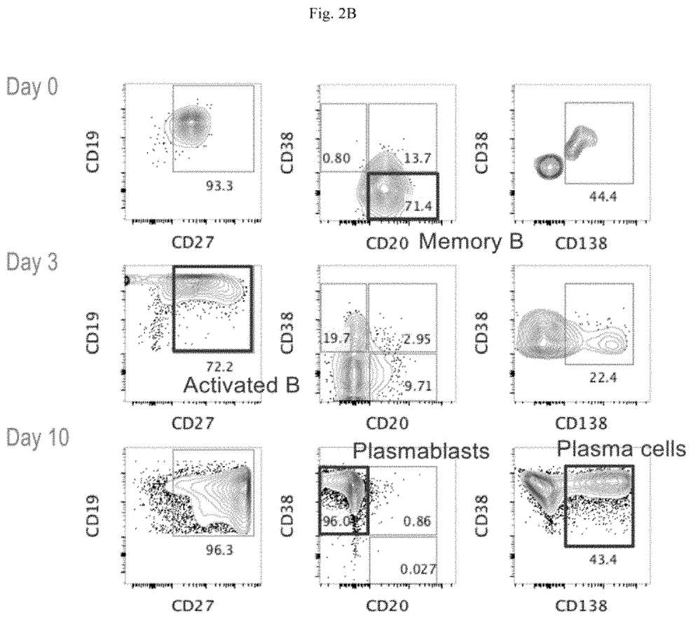

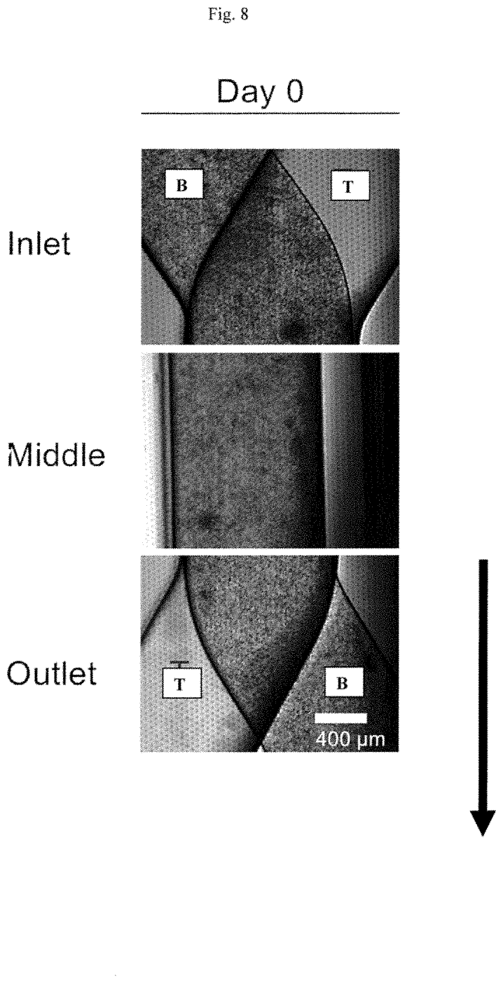

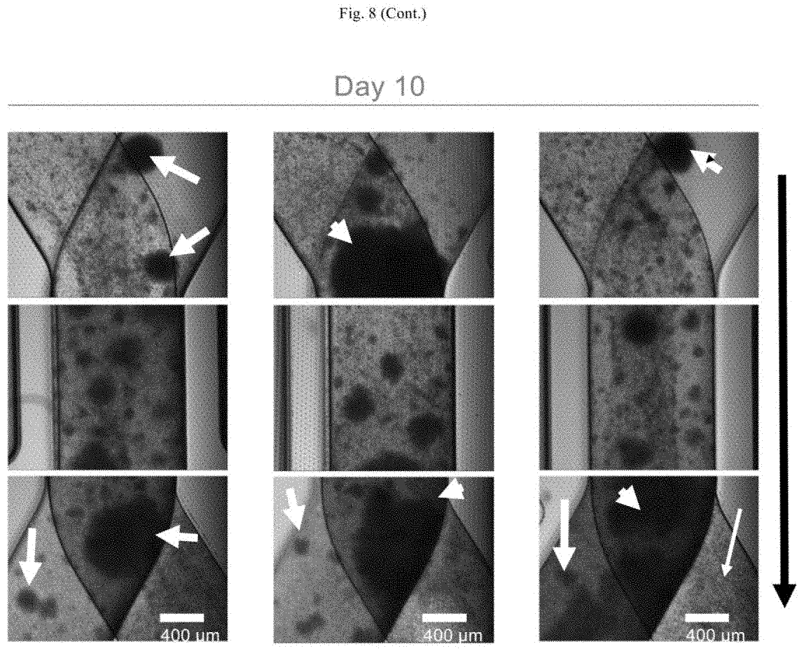

Numerous formulations and methods were compared during the development of the present inventions, using plate co-cultures of autologous PBMCs, either whole or as purified subpopulations of PBMCs, e.g. CD3−CD19+CD27+ activated B cells. Several are described herein. The following example is one exemplary protocol developed using white blood cell populations cultured in a tissue culture plate (Example A and FIG. 2) that was then used in a microfluidic device (Example B and FIG. 8), as described herein, for providing large amounts of IgG antibody within 3-10 days after initiation of cultures from the majority of tested normal human PBMCs from donors.

### Example A

The following is an exemplary timeline, procedure and types of medium used for providing soluble antibodies in plate cultures.

Exemplary methods: Total PBMCs may be isolated from whole blood using any method that yields both T cell and B cell populations. As used for this example, PBMCs were isolated from whole blood, i.e. recovered, using EasySep (StemCell Technologies).
In some embodiments, B cell populations may be purified based upon methods for negative selection for providing CD27+(CD3−CD19+) B cells using any one of known methods of purification, e.g. EasySep (StemCell Technologies); tagging CD27+ cells for sorting into a purified population for adding to plates. In some embodiments, plates may be covered by CD27 antibodies (i.e. anti-CD27) for plating CD27+ cells by washing off unattached cells.
Exemplary base B Cell Activation media: Iscove's modified Dulbecco medium (IMDM, Thermo) with 10% fetal calf serum (FCS: HIFBS; Invitrogen) and Glutamax (Thermo). Exemplary base B Cell differentiation and base B Cell maintenance media also contains: Lipid Mixture 1 (Sigma) and MEM amino acids (Thermo).
Day −1: Recover PBMCs; Day 0: Plated purified CD27+(CD3−CD19+) B cells in base activation medium (further comprising activation associated molecules IL-2, IL-21, soluble CD40L, in addition to adding a mixture of AffiniPure F(ab′)2 Fragment Goat Anti-Human IgM, Fc_{5μ} fragment specific proteins and AffiniPure F(ab′)2 Fragment Goat Anti-Human IgG, Fc_{5μ} fragment specific proteins; Day 3: Change medium to base differentiation medium (further comprising differentiation associated molecules IL-2 and IL-21); Day 6: Change medium to maintenance medium (further comprising maintenance associated molecules IL-6, IL-21, and IFN-alpha); and Day 10: observe terminal differentiation of B cells as plasma cells, in additional to other B cell types.
Observations include bright field microscopy, immunohistochemistry and flow cytometry. For obtaining single cell suspensions for labeling and flow cytometric analysis, cells are removed from plates in a manner that does not digest cell surface biomarkers tagged for immunofluorescent multichannel (multicolor flow cytometry) analysis. In some embodiments, cells may be surrounded by hydrogels as described herein, wherein the hydrogels are dissolved leaving cell surface biomarker molecules intact.
FIG. 2A shows exemplary bright field microscopic images of human B cell cultures during incubation in 2D cultures (e.g. plates) using one embodiment of a 3 step method over 10 days as described herein. Day 3 cells after initiating activation on Day 0 showing some clustering, Day 4 cells in differentiation medium showing larger and denser clusters than on Day 3, Day 6 changing to maintenance medium showing large-dark/dense clusters, and Day 10 showing large-dark/dense clusters. Starting cells are purified CD3−CD19+CD27+ human B cells.

In some embodiments, cell surface biomarkers are not restrained to merely to presence (i.e. plus(+)). As observed on flow plots, some biomarkers range from low (i.e. lo) to high (i.e. hi) as part of activation levels and differentiation status. In some embodiments, cells flowing through a cytometer are “gated” referring to an electronic separation of operator desired cell populations. In practice, an operator may identify a gate on a control screen for isolating a particular cell population. The “gated” cell population may then appear in a new flow plot. The number of selected gates may be determined by the instrument model used in the analysis. Further the types of gates may be restricted by the number of different types of florescent molecules used for tagging cells. Some flow cytometers may allow up to 12 or more different fluorescent molecules i.e. florescent channels.

Day 0: Seeding population: Staining then gating live cells for activated CD19+CD27+ cells, e.g. 93.3%, activated by isolation and preparation procedures. Gated CD19+CD27+ cells into showing CD38-CD20+ memory B cells, e.g. 71.4% of the 93.3% live B cells. Gated CD38+ B cells into CD38low+CD138low+, e.g. 44.4% of the 71.4% Memory B cells, and CD38lowCD138−, remainder, showing baseline expression of CD38 and CD138.
Day 3: Activated population: Staining then gating live cells for CD19+CD27+ cells, e.g. 72.2% activation media induced activation of B cells, showing up to 2 logs higher expression of CD27+ than at Day 0. Gated CD19+CD27+ cells into showing a loss of CD38 and CD20+ memory B cells, e.g. 71.4% of the 93.3% live B cells. Gate 71.4% of the Memory B cells into CD38+CD138+, e.g. 44.4% of the 71.4% Memory B cells.
Day 10: Differentiated population: Staining then gating live cells for CD19+CD27+ cells, e.g. 96.3% differentiation media induced differentiation of B cells, showing a higher percentage of CD27+ B cells than at Days 0 or 3. Gated CD19+CD27+ cells into showing an almost complete loss of CD38−CD20+ memory B cells, e.g. 0.027% of the 93.3% live B cells, and a large population of CD38+CD20−, e.g. plasmablasts. Gated CD38+ B cells into CD38+CD138+, e.g. 43.4% plasma cells.
FIG. 2B shows exemplary flow cytometry data of human B cells harvested in 2D cultures (e.g. plates) using one embodiment of a 3 step method over 10 days as described herein. Hydrogels were dissolved for releasing cells having intact cell surface biomarkers for use in antibody tagging for flow cytometry. Day 0 results profiled gated live cells for a CD19+CD27+ purified B cell population in turn showing the majority of cells are CD38−CD20+ Memory B cells and CD38^{lo}. Day 3 cells after initiating activation on Day 0 showing some clustering, Day 4 cells in differentiation medium showing larger and denser clusters than on Day 3, Day 6 changing to maintenance medium showing some large-dark/dense clusters, and Day 10 showing some large-dark/dense clusters. Starting cells are purified CD3-CD19+CD27+ human B cells.

1. Other Transwell/Plate Immunological B Cell Maturation Systems.

There a numerous types of B Cell maturation systems described in the literature, including several references of plate based systems, examples cited herein. However, in general these references have no demonstration of actual antibody production let alone the large amounts of IgG antibodies produced by devices and methods described and shown herein. Moreover, if there is a reference to IgG production in a publication, then it is either on a per cell basis or-as an optical density basis. When there is a result in a reference showing an amount of antibody released into culture solutions, it is a low ng/ml amount. Thus, the inventors are not aware of a system showing the replicable, high levels of IgG provided as demonstrated herein.

Further, there are commercialized transwell/plate immunological testing systems for drug candidates promoting B cell maturation and antibody production, examples include Probiogen's HuMAN and VaxDesign's MIMIC system. The Human Artificial Lymph Node (HuALN) system is based on a miniaturized, perfused (up to 2 μL/hr and up to 13.1 μL/h), bioreactor for long-term cultivation (up to 4 weeks) of human blood-derived immune cells within hydrogels. It appears in an associated publication, WO2009024595A2 to Probiogen Ag, published 2009 Feb. 26, that an “ . . . antibody response may take 2 to 21 days (IgM), or 4 to 21 days (IgG) . . . ”. Furthermore, there is no mention of bovine collagen containing hydrogels, nor the biologically active activation molecules, i.e. IL21 and CD40L. Moreover, unlike shown herein, there is no demonstration that antibodies are actually produced. If antibodies are produced there is no indication of amounts.

Another system, MIMIC® System, is based upon 96-well plastic microtiter plates or transwells, as modules, e.g. A Vascular PTE module, Lymphoid Tissue Equivalent (LTE) module, Clinical Trial in a Test Tube™, either alone or in combination. See WO2005104755. Donor immune cells were placed into the MIMIC® System both pre-vaccination and post-vaccination, with influenza specific antibody responses measured by ELISA. The mimic system turned out under 200 ng/ml of antibodies. For comparison, antibodies in circulation were measured in upwards of 100-200 ug/ml amounts.

2. Other Lymphoid Microfluidic Devices.

Numerous publications describe attempts for simulating in vitro lymph node function, including antibody production on microfluidic platforms and microfluidic organ chips. In particular, a patent publication to Ingber (WO 2018/017605), and a recent manuscript publication, -(Goyal and Ingber, 2019), describe a human Lymph Node (LN)-chip microfluidic device as another type of Organ-on-a-chip. However, neither describes specifically using bovine collagen alone or bovine collagen (i.e. Fibricol) at the concentrations of 1-2 mg/ml. Moreover, these publications do not use cytokines of the present inventions, e.g. IL-21 or activation molecules, e.g. soluble CD40L, while instead using IL-4 and CD40 agonistic antibody.

Moreover, lymph node chips in these publications in general do not show the high levels of IgG antibody production as demonstrated herein. Further, unlike demonstrated herein, activated B cells appear to become quiescent in other Lymph Node Microfluidic devices corresponding to a lack of demonstration of high level IgG production. Total immunoglobulin levels in these publications were measured using ELISA (e.g., Bethyl Biolabs, E80-104 or Mesoscale discovery, K15203D). Influenza HA-specific IgG was detected using a modified version of a previously described digital ELISA assay.

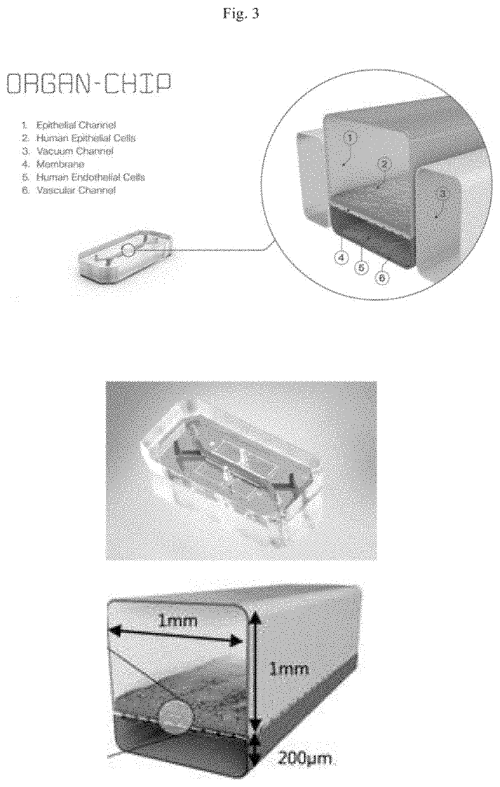

In conclusion, unlike levels in the thousands of ng/ml produced by one embodiment of an AB-Lymph node chip described herein, total IgG production by the Goyal et al., LN chip showed merely 4-12 ng/ml of IgG in effluent 6 days after exposure to IL-4 and anti-CD40 Ab. 3 days after treatment with SAC, see FIGS. 3A and 3F, respectively. Between 0.75 around 1.25 ng/ml were produced when engineered with naïve B cells and bulk T cells.

Compositions and methods described herein, in particular related to the use of bovine collagen I, an inventive B cell activation medium comprising IL-21 and CD40L, B cell differentiation medium and B cell maintenance medium, and a 2 to 3 step method of use thereof, are contemplated for use with human white blood cells regardless of the type of culture mode, e.g. 2D plate cultures, 3D plate cultures, Transwell cultures, bioreactors, or other types of microfluidic devices, etc.

B. Exemplary B Cell Differentiation in Microfluidic Devices for Antibody Production.

Although the term “Lymph Node-Chip” is used herein, there are significant differences between the compositions and methods used herein, including significantly lager amounts of IgG produced within 3-10 days, as compared to other published “Lymph Node-Chip” or “Lymphoid-Chips,” etc. Therefore, microfluidic devices provided using compositions and methods described herein may also be referred to as “Antibody Producing Microfluidic Device” or “Antibody-Chip” or “AB-Lymph Node Chip”.

Moreover, in some embodiments, Antibody-Chips are seeded with autologous isolated total PBMCs for comparing formulations and methods for providing plasma cells and IgM and IgG immunoglobulins as early as Day 5 and within 10 days after initiation of B cell activation using B cell activation media. In some embodiments, cells seeded into chips immediately undergo stimulation procedures described herein. In some embodiments, cells seeded into chips are held in maintenance medium, described herein, for several days until beginning stimulation procedures.

In some embodiments, an S-1 tall channel microfluidic device is provided, modified and used herein.

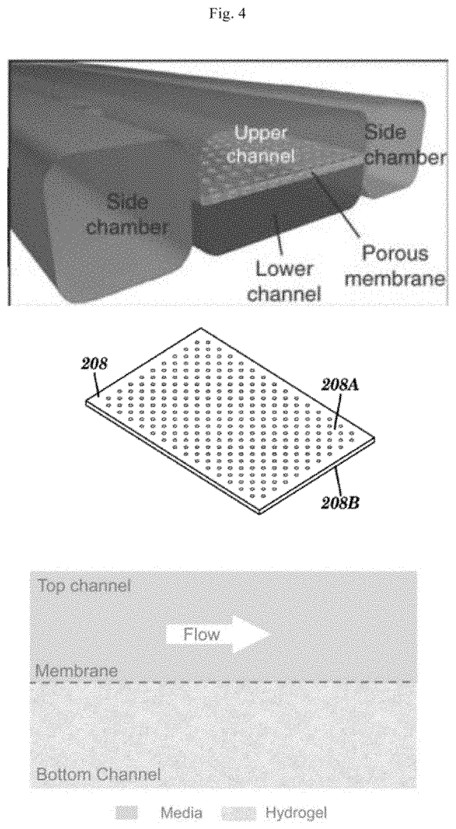

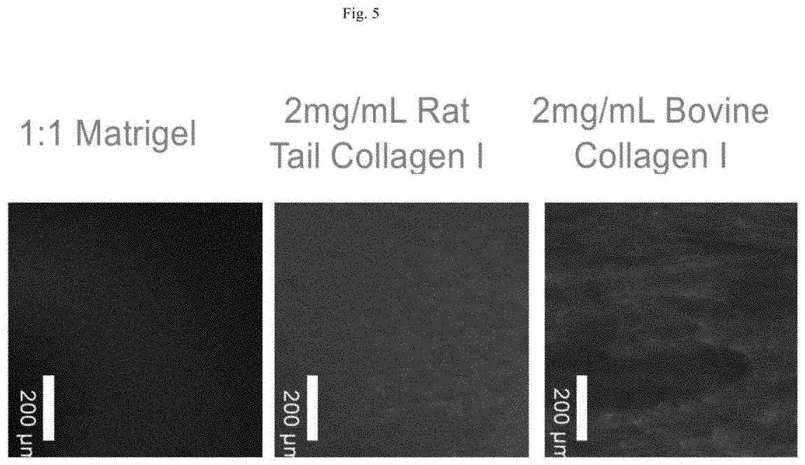

FIG. 3 shows an exemplary schematic illustration of one embodiment of a microfluidic device shown as a S1 (tall channel) organ-chip comprising upper (light) and lower (dark) cell culture microchannels with a microfabricated porous elastic membrane sandwiched in-between. In some embodiments a microdevice may also be equipped with two full-height, hollow microchambers alongside of the cell culture channels. An exemplary organ chip has: 1. Epithelial Channel; 2. Epithelial Cells, e.g. primary cells, cell lines, Caco2, primary intestinal cells, cancer cells, etc.; 3. Optional Vacuum Channel; 4. Membrane, optional stretch; 5. Endothelial Cells e.g., human Intestinal HIMEC or iHIMEC, etc.; and 6. a Vascular Channel. See, WO 2010/009307A2, herein incorporated by reference in it's entirety.
FIG. 4 shows an exemplary schematic illustration of one embodiment of a S1 microfluidic device (upper) with an enlarged schematic of a membrane 208, upper side 208A, lower side 208B, separating two channels. Unlike other organ microfluidic devices, for lymph node-chips producing antibodies, endothelial cells were not typically seeded into either of the channels. In fact, the presence of endothelial cells on-chip during variable testing interfered with antibody production. For lymph node-chips producing antibodies, hydrogels were flowed through the lower channel for filling space within these channels. For testing hydrogel integrity, there was no direct flow in the lower channel while a constant flow was provided in the upper channel. Grey solid depicts medium in the upper channel under flow while grey speckled depicts the solidified hydrogel filling the lower channel under the membrane that is not under direct fluid flow through the lower channel.

### Example A

Antibody Production from an Isolated B Cell Population Using a S1 Tall Channel Chip.

The following is an exemplary method for preparing chips, preparing hydrogels and seeding/loading white blood cells into chips.

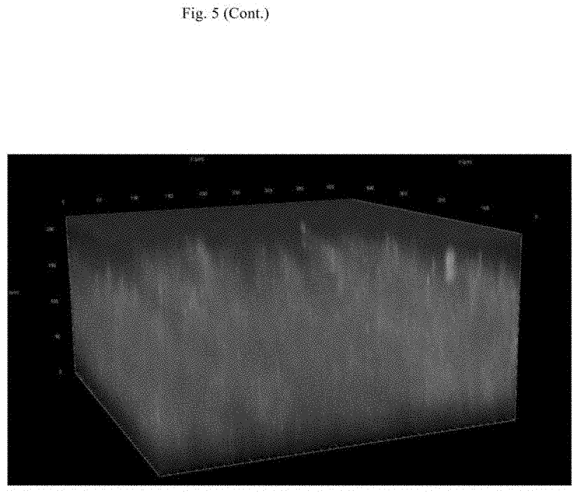

FIG. 5 shows an exemplary illustration of one embodiment of a S1 (tall channel) chip where the lower channel was coated with comparative formulations of hydrogels stained for gel proteins. 1:1 Matrige®:bovine collagen I (Fibricol®); 2 mg/mL Rat Tail Collagen I; 2 mg/mL bovine Collagen I (Fibricol®). Lower image shows a stained hydrogel within a channel.

Interior surfaces of microfluidic devices were activated, e.g. using methods corresponding for using ER-1. FibriCol® type I bovine atelocollagen solution protein from bovine hide was mixed 1:1 with Matrigel®.

This liquid hydrogel solution is prepared at around 4° C. Approximately 2 million cells are mixed into the cold hydrogel solution then flowed into the bottom channel of one embodiment of a two channel chip then polymerized by heating in a 37° C. incubator for one hour.

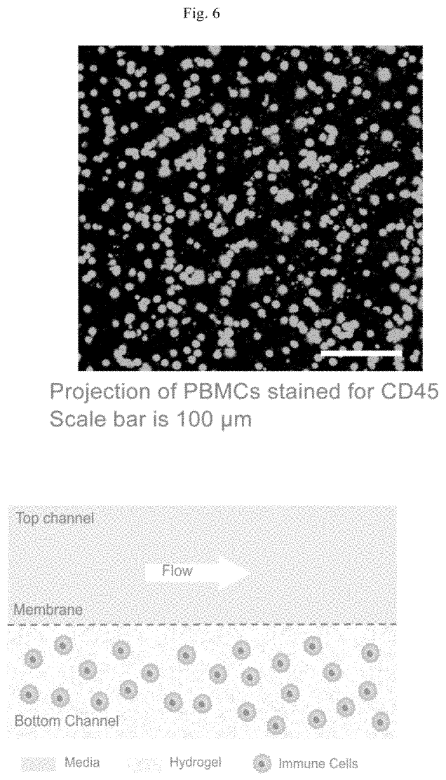

FIG. 6 shows an exemplary isolated peripheral white blood cell population immunostained for CD45 (upper white cells) demonstrating a range of sizes and shapes. Scale bar is 100 μm. In some embodiments, the entire CD45+ population resulting from isolation produces is used as described herein. In some embodiments, CD19+CD27 white blood cells are purified into a population for use as described herein. An exemplary illustration is shown (lower) of one embodiment of an S1 organ-chip configuration where the bottom channel is filled with a hydrogel—white blood cell mixture (e.g. PBMCs embedded in a hydrogel as described herein) for producing antibodies. During co-culture, the top channel, under media flow, grey, is separated by a membrane from the Bottom Channel not undergoing direct flow during incubation in culture medium.

Chips were then fluidically connected to a biological culture chamber that in turn was placed into a perfusion manifold under 30 uL/hour flow in the top channel and 0 uL/hour flow in the bottom channel which is containing the solidified hydrogel and embedded cells. Fresh media is continuously flowed through the upper channel without recirculation. At least 3 different types of media were used over time in a two to three step procedure as described herein for at least a 10-day culture period, which may be termed “under a differentiation protocol” of the present inventions.

In one embodiment, Day −1: Recover PBMC. Day 0: Plate CD19+CD27+ B cells in activation medium containing activation associated molecules IL-2, IL-21, CD40L, and F(ab′)₂ anti-IgG/IgM); Day 3: Change to differentiation medium containing IL-2 and IL-21, lacking CD40L, and F(ab′)₂ anti-IgG/IgM); Day 6: Change to maintenance medium containing IL-21, lacking IL-2, CD40L, F(ab′)? anti-IgG/IgM) further containing IL-6 and IFN-alpha; and Day 10: End of assay.

Chips were imaged at least days 0, 3, 6, and 10. Some of the duplicate chips were used for flow cytometry analysis by digesting hydrogels then flushing cells out of the chips for immunostaining/histochemical staining and analysis. In some embodiments, upper channel cells were released from the membrane and channel surfaces for immunostaining/histochemical staining and analysis. For flow cytometry analysis; dissolving out hydrogels and detaching cells from upper channels is done for releasing single cells having intact membrane molecules for tagging with B cell biomarkers.

The following provide flow cytometry results of cells harvested from hydrogels in the lower channel.

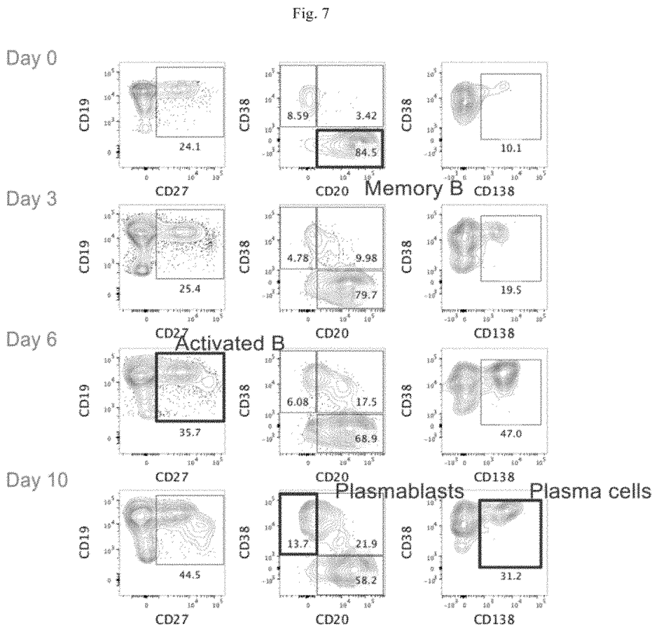

Day 0: Seeding population: Staining then gating live CD19+CD3− cells for activated CD19+CD27+ cells, e.g. 24.1%, activated by isolation and preparation procedures. Gated CD19+CD27+ cells into showing CD38−CD20+ memory B cells, e.g. 84.5% of the live of the live activated. Gated CD38+ B cells into CD38+CD138low+, e.g. 10.1% of the CD19+CD27−CD38+, showing baseline expression of CD38+CD138+ plasma cells.
Day 3: Activated population: Staining then gating live CD19+CD3− cells for activated CD19+CD27+ cells, e.g. 25.4% activation media induced activation of B cells. Gated CD19+CD27+ cells into showing CD38 and CD20+ memory B cells, e.g. 79.7% of the live activated B cells. Gate CD38+ cells showing CD38+CD138+, e.g. 19.5% of plasma cells.
Day 6: Differentiated population: Staining then gating live CD19+CD3− cells for CD19+CD27+ cells, e.g. 35.7% differentiation media induced differentiation of B cells, showing a larger percentage of cells expressing of CD27+ than at Day 0 or Day 3. Gated CD19+CD27+ cells showing CD38−CD20+ memory B cells, e.g. 68.9%. Gated CD38+ B cells showing CD38+CD138+, e.g. 47.0% of the 71.4% plasma cells.
Day 10: Differentiated population on Maintenance media: Staining then gating live CD19+CD3− cells for CD19+CD27+ cells, e.g. 44.5%. Gated CD19+CD27+ cells into showing CD38−CD20+ memory B cells, e.g. 58.2%, and a large population of CD38+CD20− e.g. 13.7%, plasmablasts. Gated CD38+ B cells into CD38+CD138+, e.g. 31.2% plasma cells.
FIG. 7 shows exemplary flow cytometry data of B cells immunostained for activation and maturation markers demonstrating B Cell Differentiation in 3D cultures (e.g. one embodiment of a Lymph Node-Chip). Live CD19+CD3− cells derived from a CD19+CD27+ WBC population profiling (outlined in thick black boxes) CD38-CD20+ memory B cells, activated CD19+CD27+ cells, CD38+CD20− plasmablasts and CD38+CD138+ plasma cells. GC B cells are CD38+CD20+.

### Immunoglobulin Results:

100 uL of supernatant was collected from the outlet of the top channel in the perfusion manifold analyzed using commercial IgM and IgG ELISAs. Each line represents an individual chip.

IgM increases between Day 6-10 with a max of 40,000 ng/mL at Day 7.

IgG production increases between Day 4-10 with a max of 380,000 ng/mL at Day 10.

The production of secreted IgG indicates class switching and plasma cell differentiation has occurred on the S1 chip.

In some embodiments, highest titers of IgM are found on Day 7. In some embodiments, sharply increasing titers of IgG are measured between Day 6 and Day 7. In some embodiments, titers of IgG are above 100,000 ng/ml by Day 7.

### Day 10 Bright Field Images

The middle of the bottom channel is full of cells, appearing black during images.

The gel contains clusters of cells with the most cells in the middle and large clusters at the outlet.

Both the top channel outlet and bottom channel outlet were full with cells.

FIG. 8 shows exemplary bright field microscopic images of PBMCs in a microfluidic chip undergoing one embodiment of a differentiation protocol. Day 0—upper image. D10-lower images, each panel showing images from different donors. B=bottom channel. T=top channel. The lower gel is full of cells including large clusters of cells indicative of germinal centers (GCs) with the most individual cells in the middle and large clusters towards the outlets. The middle of the bottom channel is full of cells, appearing black in these images. Both the top channel outlet and bottom channel outlet were full with cells. Cells that migrated into top channels formed large cell clusters indicative of germinal centers (GCs).

Surprisingly, explosive huge dark cell clusters within microfluidic channels throughout hydrogels were observed within the AB-Lymph Node Chips that were consistently found across several from different donors. Even more surprising, large clusters of cells in addition to smaller clumps and individual cells in the upper channels by Day 10 were observed for every donor tested.

Therefore, flow cytometry analysis was done on Day 10 comparing cells in the upper channel to the cells harvested from the lower channel to identify which cell types migrated into the upper channel.

Day 10 Flow cytometry analysis.

Because there was a large collection of cells in the top channel outlet based on microscopy, we collected the top channel for flow cytometry analysis.

Gated on live cells.

CD138 expression (right panel) is gated on Plasma Cells (CD20−CD38+).

14-20% of activated B cells are plasma cells.

15-30% of plasma cells express CD138.

Majority of the cells in the top channel are activated and differentiated into plasma cells (CD20−CD38+). However, the CD138 expression is low for Day 10 of the differentiation protocol.

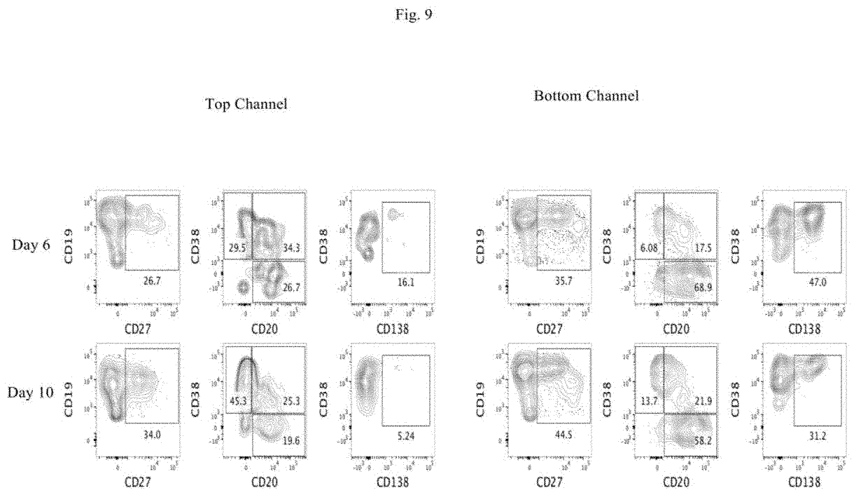

FIG. 9 shows exemplary flow cytometry data of B cells derived from CD19+CD27+ B cells immunostained for activation and maturation markers demonstrating populations of B Cells between top and bottom channels at Days 6 and 10.

### Example B

Antibody Production from Total (Whole) PBMCs Using a S1 Tall Channel Chip.

3 million PBMC were encapsulated in 15 uL of 60% Matrigel+0.45 uM Fibricol in the bottom channel of the S1 chip.

Flow rate: 30 uL/hr in the top channel, 0 uL/hr in the bottom channel.

### Day −1: Recover PBMC.

Day 0: Plate CD27+ B cells in activation.
medium (IL-2, IL-21, CD40L, and F(ab′)2 IgG/IgM).
Day 3: Change to differentiation medium (IL-2 and IL-21).
Day 6: Change to maintenance medium (IL-6, IL-21, and IFN-alpha).
Day 10: Terminate differentiation.

D0-D3 Media:

20 U/mL IL-2

50 ng/mL IL-21

50 ng/mL CD40L

10 ug/mL F(ab′)2 anti-human IgM and IgG

D3-D6 Media:

20 U/mL IL-2

50 ng/mL IL-21

D6-D10 Media:

10 ng/mL IL-6

50 ng/mL IL-21

100 U/mL IFN-a

### Flow Cytometry

Day 10 Flow cytometry analysis.

Because there was a large collection of cells in the top channel outlet based on microscopy, we collected the top channel for flow cytometry analysis.

Gated on live cells.

CD138 expression (right panel) is gated on Plasma Cells (CD20−CD38+).

59-62% of activated B cells are plasma cells.

8-12% of plasma cells express CD138.

Majority of the cells in the top channel are activated and differentiated into plasma cells (CD20−CD38+). However, the CD138 expression is low for Day 10 of the differentiation protocol.

There's a higher frequency of CD138 expression in the bottom channel.

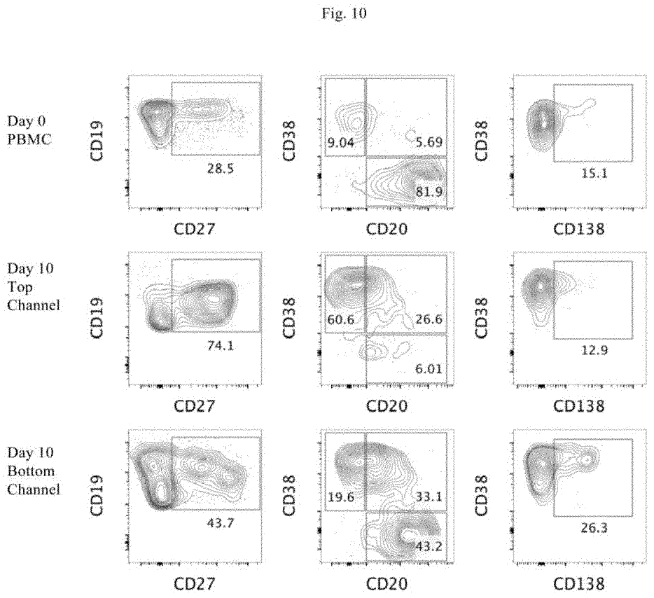

FIG. 10 shows exemplary flow cytometry data of B cells derived from PBMCs immunostained for activation and maturation markers demonstrating populations of B Cells between top and bottom channels at Days 0 and 10.

100 uL of supernatant was collected from the outlet of the top channel in the pod.

IgM increases between Day 6-10 with a max of 40,000 ng/mL at Day 7.

Exemplary IgM and IgG measured in effluent from the top channel. IgM over 10 days shows little IgM with some expressed around day 6 to day 10, e.g. less than 50,000 ng/ml. IgG over 10 days shows little initial IgG with some expressed after day 4 up to day 10. Between day 4 and day 5 amounts are relatively low however after day 5 some chips show greater than 50,000 ng/ml and after day 6 some chips show concentrations greater than 100,000 ng/ml up to 380,000 ng/ml. Each line represents an individual chip. 200 uL was collected from the top channel outlet and allowed to accumulate over the course of D1-3, D4-6 and D6-10. For example D10 is the accumulated concentration from D6-D10. The activation protocol is the full non-specific stimulation using anti IgG/IgM, CD40L, IL-2, IL-21.

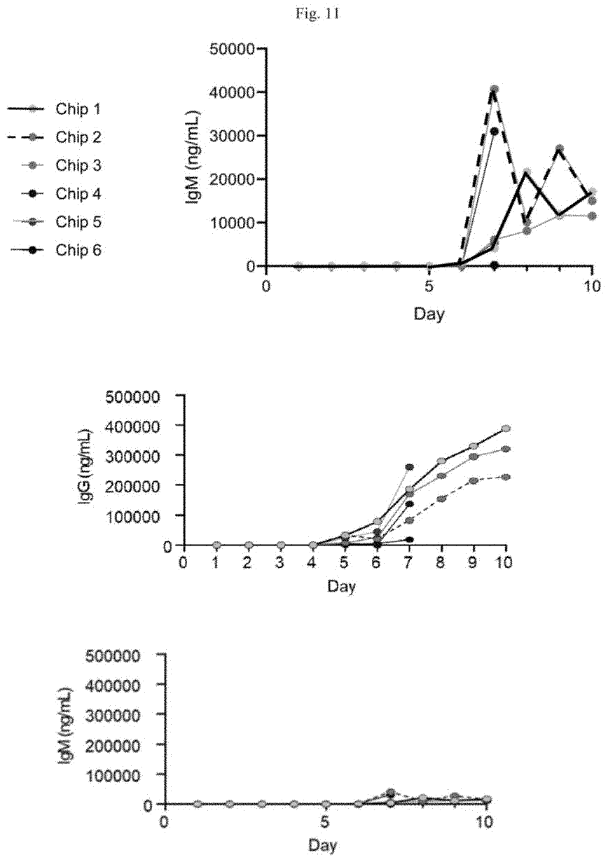

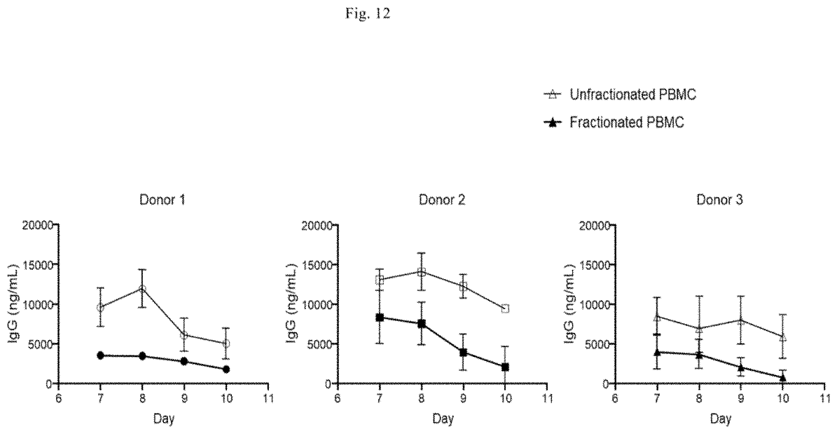

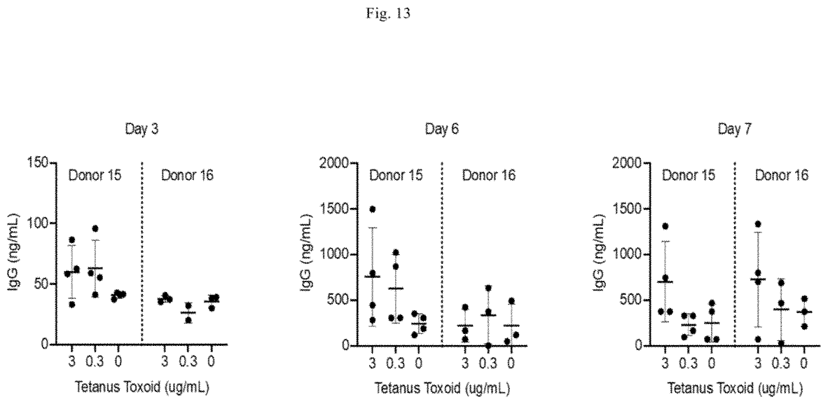

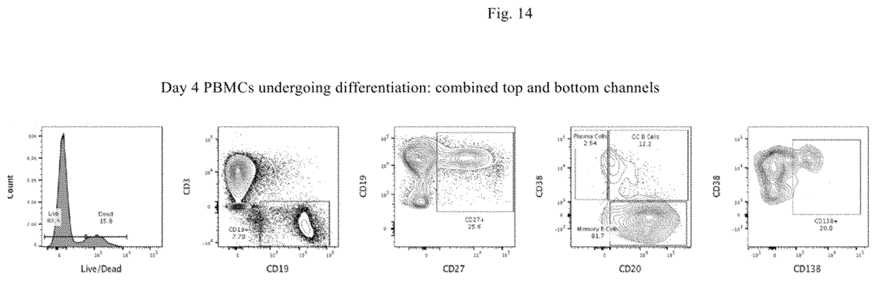

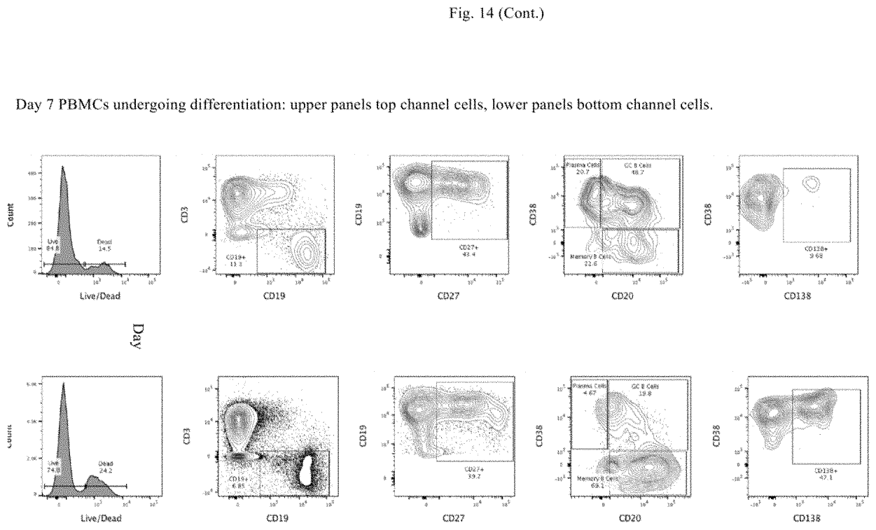

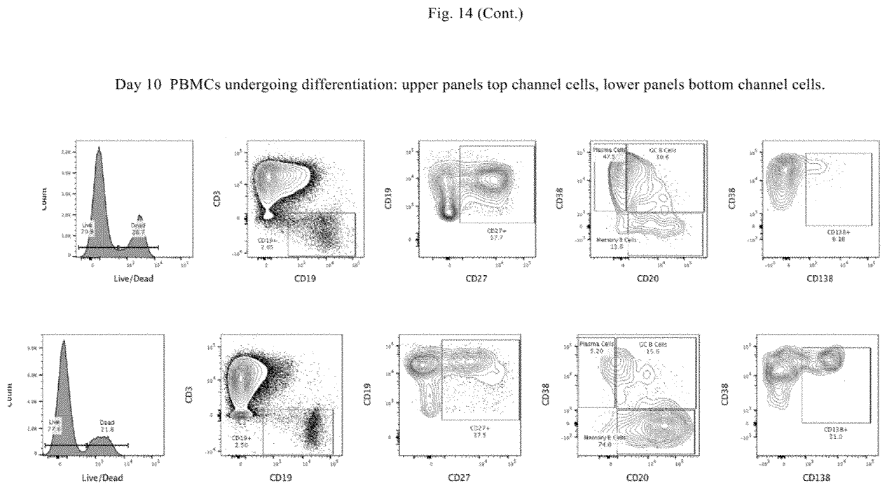

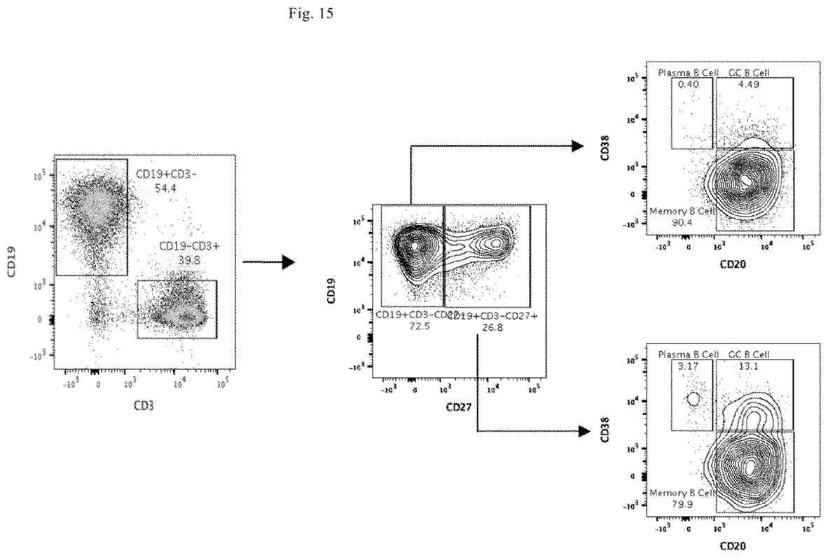

FIG. 11 shows exemplary IgM and IgG measured from top channel effluent. IgM was detected up to 40,000 ng/ml. IgG was detected in concentrations greater than 100,000 ng/ml up to 380,000 ng/ml. Each line represents an individual donor/chip. A second graph of IgM is provided using the same scale as the IgG graph for a direct comparison of amounts. Effluent samples (200 ul) were collected every 24 hours then accumulated-combined, e.g. Day 10 amounts were measured in samples collected on days 6, 7, 8, 9 and 10. A three step method using B cell activation, differentiation and maintenance media was used as described herein.
Fractionated and unfractionated PBMCs in the Lymph Node-Chip: 3 million unfractionated PBMC or fractionated B and T Cells (1:1 ratio) were encapsulated in 60% Matrigel+0.45 μM Bovine Collagen I. Supernatant collected from the top channel was analyzed using IgG ELISAs. Unfractionated PBMC chips produce higher IgG despite a lower frequency of B cells compared to fractionated PBMC chips.
TOP CHANNEL OUTLET WAS ASPIRATED EVERY 24 HOURS AFTER COLLECTION. This means that each data point indicates the amount of IgG made in a 24 hour window. The activation protocol is the full non specific stimulation using anti IgG/IgM, CD40L, IL-2, IL-21.
FIG. 12 shows exemplary amounts of IgG measured in upper channel effluent after non-specific stimulation compared between fractionated (CD19+CD27+ purified from PBMCs), lower dark symbols and lines, and unfractionated (total) PBMCs, upper light symbols and lines, in one embodiment of a Lymph Node-Chip. A three step method using B cell activation, differentiation and maintenance media was used as described herein.
Unfractionated PBMC seeded in activation media with a Tetanus Toxoid test substance with activation molecules consisting of CD40L+IL-21.
As demonstrated in FIG. 13, at least 2 types of immunoglobulin responses to exposure to an antigen are observed, one where the donor's B cells showed a memory response with short term antibody production and another where the other donor's B cells showed a developing antibody response after little or no short term antibody response.
FIG. 13 shows an exemplary comparison of IgG measured in upper channel effluent after antigen-specific stimulation, using Tetanus Toxoid (TT), of unfractionated (total) PBMCs. A three step method using B cell activation, differentiation and maintenance media was used as described herein, however with the lack of IL-2 and goat anti-human Fab2 fragments with or without TT in the stimulation media.
CD19+CD3−CD27+ cells show a higher percentage of plasma cells and germinal center cells than in the corresponding CD27− population which has a higher number of memory B cells.
In some embodiments, percentages of activated B cells used for determining whether a test substance is antigenic to the autologous white blood cells.
In some embodiments, percentages of plasmablast cells are used for determining whether a test substance is antigenic to the autologous white blood cells. In some embodiments, percentages of plasma cells are used for determining whether a test substance is antigenic to the autologous white blood cells.
FIG. 11 shows exemplary flow cytometry data of B cells derived from PBMCs immunostained for activation and maturation markers demonstrating populations of live gated B Cells at Day 4 combined top and bottom channel, Day 7 and Day 10 top vs. bottom channels.
FIG. 15 shows exemplary flow cytometry data of a replicate Lymph Node chip seeded with total PBMCs immunostained for activation and maturation markers demonstrating populations of live gated B Cells at Day 4 combined top and bottom channel, Day 7 and Day 10 top vs. bottom channels. Nonspecific stimulation using a 2 step procedure as described herein.

In summary, after co-culturing of with purified CD3−CD19+CD27+ B cells and isolated total (whole) PBMCs in a series of activation, differentiation and maintenance media, a large population of CD3−CD19+CD27+CD20−CD39+CD138+IgD− cells identified as plasma cells were produced in both plate cultures and on a microfluidic chip, i.e. using the methods described in Example A and Example B.

### Example C

Antibody Production from Tonsil Cells Using a S1 Tall Channel Chip.

In some embodiments, compositions and methods used on total PBMCs were applied to single cell suspensions of CD45+ white blood cells derived from tonsil lymphoid tissue biopsies.

In some embodiments, compositions and methods used for providing CD19+CD27+ B cells were applied to tonsil lymphoid tissue biopsies.

Peripheral blood mononuclear cells (PBMC) were isolated directly for tonsil biopsy cell suspensions by density gradient centrifugation over Histopaque-1077 (Sigma-Aldrich; Steinheim, Germany).

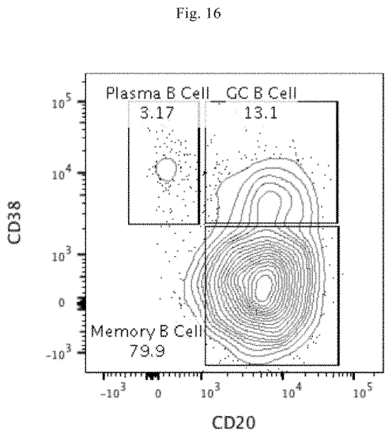

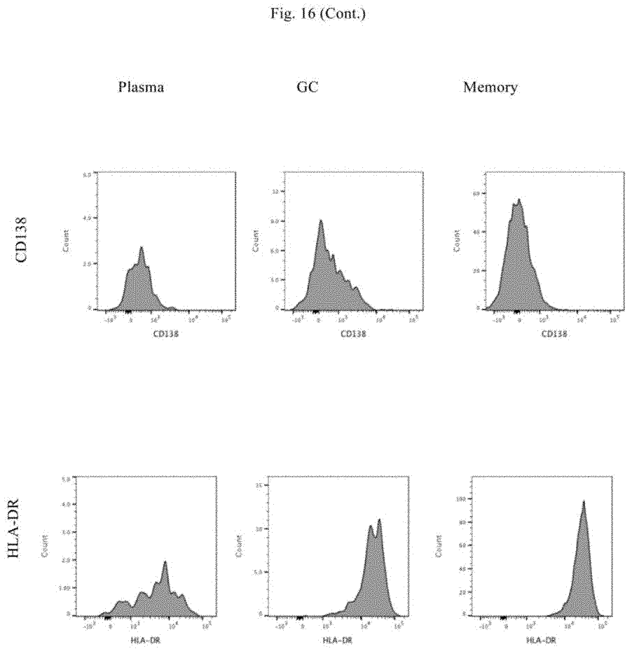

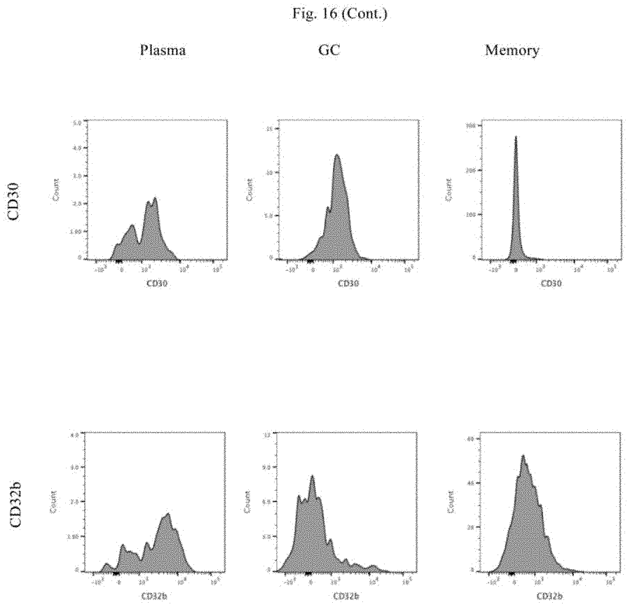

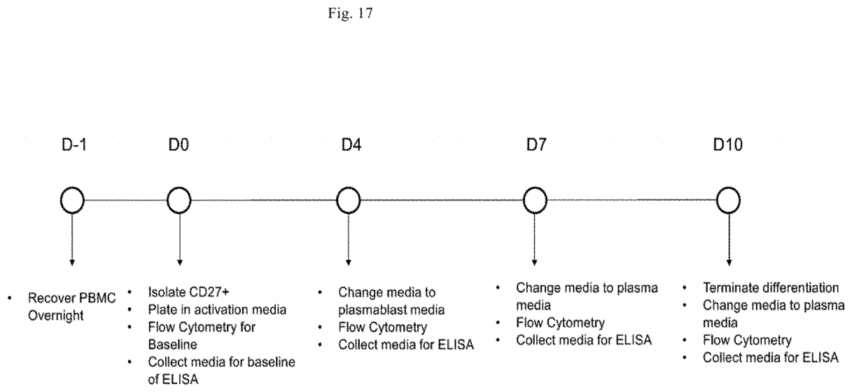

FIG. 16 shows exemplary flow cytometry staining and a cell gating strategy for evaluating tonsil white blood cell types seeded in plates and microfluidic chips for undergoing a 3 step culture as described herein. 1) CD19+CD3− cells were gated into CD19+CD3−CD27− cells and CD19+CD3−CD27+ cells. 2) CD19+CD3−CD27− cells were gated into CD38+CD20− plasma cells, CD38+CD20+ germinal center cells and CD38−CD20− memory B cells.3) CD19+CD3−CD27+ cells were gated into CD38+CD20− plasma cells, CD38+CD20+ germinal center cells and CD38−CD20− memory B cells.
FIG. 17 shows exemplary flow cytometry staining of CD19+CD3−CD27+ tonsil cells gated into plasma cells, germinal center cells and memory cells comparing CD138, HLA-DR, CD30 and CD32b cell surface expression levels.

### Example D

Antibody Production from Tonsil Cells in Combination with Thf Cells.

In some embodiments, CD14+CD16− monocytes were purified by negative selection via immunomagnetic separation using EasySep monocyte isolation kits with CD16 depletion (Stemcell Technologies; Grenoble, France) according to the manufacturer's instructions.

In some embodiments, isolated monocytes were cultured at a density of 1×106 cells/ml in RPMI1640 supplemented with 10% fetal calf serum (FCS), 1% glutamine, 1% HEPES buffer, 1% non-essential amino acids (all from Sigma-Aldrich).

In some embodiments, follicular helper T cell (TFH cell) differentiation protocols are provided herein.

Gradients within Hydrogels Comprising White Blood Cells.

In some embodiments, a two membrane 3 channel chip is used. The 2-membrane chips do not undergo membrane stretch, therefore the vacuum channels and ports are not used for applying a vacuum.

In some embodiments, S-1 tall microfluidic chips are configured for an inlet and outlet for the center channel. In some embodiments, the inlet and outlet for the center channel connect to vacuum ports for flowing hydrogel and cells into a lower channel.

In some embodiments, S-1 tall microfluidic chips are configured to eliminate vacuum ports.

In some embodiments, liquid hydrogels are flowed into the center channel in a manner for reducing spill over through separating membranes into upper and lower channels. In some embodiments, after flowing a liquid hydrogel into the center channel, the inlet and outlet for the center channel are blocked, e.g. a piece of PDMS. In some embodiments, match connections to vacuum ports. In some embodiments, the inlet and outlet for the center channel are located to match fluidic connections

For verification of the presence and amount of gradients, a two membrane chip having a center channel filled with solidified hydrogel proteins, has fluorescent molecules in solution flowed through either the lower or upper channel for visualizing the formation of a gradient, and then for visualizing the concentration levels throughout the gradient. After gradient information in obtained, then a two membrane chip having a center channel filled with solidified hydrogel proteins comprising white blood cells, has fluorescent molecules in solution flowed through either the lower or upper channel for visualizing the formation of a gradient, for visualizing the concentration levels throughout the gradient and then for observing cell placement within the hydrogel within the gradient concentration levels and then to observe cell migration in the presence of flow and a molecular gradient.

In other embodiments, a cellular gradient formed in response to a molecular gradient is observed by antibody staining for a particular cell type, as described herein.

### Example E

Induction of Gradients within Hydrogels Comprising White Blood Cells.

In some embodiments, an AB-Lymph Node microfluidic device has two fluidic channels, one on top, the other on the bottom, of at least one or more internal channels, each separated form the other by a porous membrane. In one embodiment, an AB-Lymph Node microfluidic device is a dual membrane device, such as described herein, having at least 3 microchannels in fluidic communication with each other in as stacked configuration, wherein the center channel, in some embodiments, has an inlet and an outlet port. Such dual channel microfluidic devices are contemplated for inducing additional migration of B cells into forming clusters, and in some embodiments, for extending viability of memory B cells for use in multiple stimulation assays.

In some embodiments, human peripheral blood mononuclear cells are isolated from whole blood, as described herein. In some embodiments, white blood cells are cultured as described herein.

In some embodiments, human B cell attracting chemokines including but not limited to CXCL13 (recombinant human (rh)CXCL13), CCL19, CXCL12 and CCL21, and CCR7 (Perprotech (Rocky Hill, N.J., USA)). In some embodiments, a chemotactic gradient is formed. In some embodiments, a chemotactic gradient is formed within the hydrogel layer. In some embodiments, a chemokine is added to a medium. In some embodiments, a chemokine is added to a B cell medium. In some embodiments, a chemokine is added to a B cell stimulation medium as described herein. In some embodiments, a chemokine is added to a B cell differentiation medium as described herein. In some embodiments, a chemokine is added to a B cell maintenance medium as described herein. In some embodiments, a chemokine is contemplated for use in a dose-dependent manner for stimulating chemotactic activity by increasing the amount of migrating B cells on chip. In some embodiments, a mixture of B cell attracting chemokines is used. In some embodiments, a B cell attracting chemokines is contemplated for further analyzed using neutralizing antibodies.

In some embodiments, recombinant human (rh)CXCL13 is added to B cell stimulation medium as described herein. In some embodiments, addition of recombinant human (rh)CXCL13 is used for determining whether there are endogenous differences in the migratory potential of B cells from an individual human donor. In some embodiments, chemokines were added in media at 50 ng/ml, 100 ng/ml, 200 ng/ml up to 500 ng/ml.

In some embodiments, the chemokines are added into one fluidic channel for creating concentrations gradient from one side of the hydrogel to the other. In some embodiments, one chemokine is added to one fluidic channel while a second chemokine is added to a second fluidic channel creating an opposing concentration gradient. Readouts include determining the size and color of cluster formation; immunofluorescent labeling within the hydrogel for determining where certain tagged cells migrate in response to a chemokine gradient.

### III. Exemplary Development of Compositions and Methods for Providing Antibody Producing Microfluidic Devices.

As described herein, compositions and methods were developed for providing a lymph node chip as an antibody producing device that recapitulates in vivo biology of mature B cell activation and differentiation into antibody producing plasmablasts and plasma cells. It is not intended to limit the type of microfluidic lymph node chip as an antibody producing device. Indeed, a microfluidic lymph node chip may not have a membrane, such that fluid flow is through a single solid hydrogel filled channel. In some embodiments, a microfluidic lymph node chip may have a single membrane separating two channels, one or more of which is filled with a solid hydrogel. In some embodiments, a microfluidic lymph node chip may have two membranes separating three channels, e.g. a dual membrane microfluidic lymph node chip. In some embodiments, a dual membrane microfluidic lymph node chip has an inlet and outlet for a center channel for flowing a liquid hydrogel, with or without white blood cells, into the center channel. After which, these inlets and outlets are sealed off during incubation. In some embodiments, dual membrane chips are adapted for use in culture devices.

Numerous variables were tested during the development of the present inventions. In some embodiments, 2D protocols (e.g., as obtained from or modified from publications) were tested in 3D hydrogels in plate cultures, including but not limited to hydrogels of the present inventions. In some embodiments, optimization testing was done in plate cultures prior to testing in microfluidic devices. In some embodiments, optimization testing was done in microfluidic devices, wherein channels containing hydrogels were under flow for hydrogel testing. In some embodiments, optimization testing was done in microfluidic devices, wherein channels containing hydrogels were not under flow while adjacent channels were under flow for hydrogel testing.

In some embodiments, staining of hydrogels for observing performance over time, e.g. signs of degradation under flow, e.g. immunofluorescence observations, were optimized. In some embodiments, digestion protocol for single cell suspension e.g., reduced digestion time in order to maintain cell surface markers for use in identifying cell types by immunofluorescent antibody staining for flow cytometry.

In some embodiments, stromal cells were added to the hydrogels in the lower channel, however under the conditions tested these cells degraded the hydrogels over time. In some embodiments, flow rates were tested, including but not limited to 30 uL/hr flow and 60 uL/hr flow. In some embodiments a flow rate of 60 uL/hr flow produced higher levels of antibodies. In some embodiments, differentiation was occurring when amounts of cytokines added to the medium was reduced.

Thus in general, plate cultures and microfluidic devices producing antibodies were developed as described herein providing B cell populations determined to be phenotypically differentiated using a flow cytometry panel comprising cell surface biomarkers including but not limited to: CD3-CD19+CD27+CD20-CD38+CD138+IgD−.

A. Creating a Lymph-Node-On-Chip for Immunogenicity Testing of Molecules: High Impact Antibody Production: Antigen Specific Endogenous Antibody Producing Microfluidic Device.

As described herein, at least three different differentiation protocols were tested for providing differentiating B cells in microfluidic devices. In some nonlimiting embodiments, creation of compound specific data is contemplated in order to gauge utility of systems for test molecule immunogenicity resulting in antibody production in an AB-Lymph-Node-on-Chip of the present inventions.

As described herein, at least three major developments, i.e. highlights, were made during the development of an antibody producing microfluidic device. Such highlights resulted from extraneous back and forth testing of the following variables aimed at providing compositions and methods (i.e. optimization of variables) for producing high levels of antibodies within the device that is harvested from effluent fluids flowing out of these microfluidic devices. Variables in no particular order of priority that underwent extensive testing include but are not limited to: gel coatings for attaching non-adherent white blood cells within the device; co-culturing specific populations of human white blood cells within a microfluidic device; cytokine-based differentiation media; proliferation media; maintenance media; flow rates; etc. Highlights include but are not limited to testing for optimal cytokine differentiation methods identifying populations of human white blood cells containing antibody producing cells, developing a gel coating (B cell coating) that allows longer survival of gel integrity under flow within the device over time further allowing increased viability of human immune cells co-cultured within the gel and undergoing a differentiation treatment—as compared to other types of gels, resulting in the production of large quantities of immunoglobulin harvested from effluent fluids.

1. Exemplary Gel Coatings for Attaching Non-Adherent White Blood Cells within the Device.

Observations of solidified hydrogels comprising rat tail collagen located in the lower channel without flow while under upper channel flow showed peeling and loss of attachment proteins over time. Thus, several types of hydrogel coatings and surfaces without hydrogels were compared under flow. Surprisingly, hydrogels comprising bovine collagen I gels comparatively showed a longer life-span than other hydrogels. In some embodiments, a commercial source of bovine collagen as Fibricol was used. In some embodiments, gel survival is evaluated by observing which gel coating survives better after staining gel proteins using known immunofluorescent compounds.

Moreover, after comparing viability of human white blood cells cultured on different species of collagen sources, including commonly used rat-tail collagen, it was surprising to discover that use of a species specific bovine collagen resulted in higher viability of human white blood cells. However, it was also discovered that using higher percentages of collagen resulted in lower antibody production unless mixed with Matrigel during the differentiation procedure. In fact, it was discovered that at least some Matrigel is needed during the differentiation procedure for high level antibody production.

As demonstrated herein, the use of bovine collagen I, alone, and in mixtures with Matrigel, provided longer lasting hydrogels, i.e. increased longitivity under flow conditions. Thus, in preferred embodiments, bovine collagen I (e.g. Fibricol), is used instead of rat tail collagen alone under certain conditions and in mixtures with Matrigel.

2. Exemplary Compositions and Methods Tested Herein Producing Antibodies in Microfluidic Device Effluent Using Recombinant CD40L.

At least three published protocols were modified for use as described herein. One is termed Jourdan Approach or Jourdan method referring to methods using a soluble recombinant CD40L, another is termed Cocco approach, or Cocco method referring to the use of CD40L expressing feeder cells, and the third is termed Ugolini approach, or Ugolini method referring to creating FHC T cells derived from PBMCs for use in methods as described herein. In some cases, examples of methods for obtaining starting white blood cell populations for use herein are also described in these publications.

The following Examples demonstrate that hydrogel coatings and differentiation methods affect the amounts of IgG antibody production.

### Example F

In some embodiments, IgG antibody production was measured after activation and differentiation using modified methods, specifically using soluble CD40L. See, Jourdan, et al., “An in vitro model of differentiation of memory B cells into plasmablasts and plasma cells including detailed phenotypic and molecular characterization.” Blood. 114(25): 5173-5181. 2009.

The following is a method described in Jourdan, et al.. CD2+ cells (e.g., mainly T cells, natural killer (NK) cells and circulating monocytes and DCs) were removed from peripheral blood cells from healthy volunteers using anti-CD2 magnetic beads (Invitrogen, Cergy Pontoise, France). Negatively selected cells were then tagged and sorted for providing a purified CD19+CD27+ memory B cell (MBCs) population by FACSAria with around a 95% purity. These purified B cells were plated at 1.5×10/ml and cultured in a 3-step- and 10-day (D) culture in a 6 well flat-bottomed culture plate for providing intermediate cells—activated B cells (actBCs) and plasmablasts (PBs). Iscove's modified Dulbecco medium (IMDM, Invitrogen) and 10% fetal calf serum (FCS), supplemented with 50 μg/ml human transferrin and 5 μg/ml human insulin (Sigma).

Step 1. The best result, i.e. a 6.1-fold amplification, was achieved using activations by soluble recombinant CD40L (sCD40L) and CpG oligodeoxynucleotide 2006 (ODN) and the IL-2+IL-10+IL-15 cytokine combination. CD40L transfectant or sCD40L was used to trigger CD40 activation. IL-2 (20 U/ml), IL-4 (50 ng/ml), IL-10 (50 ng/ml) and IL-12 (2 ng/ml). IL-2 (20 U/ml), IL-10 (50 ng/ml) and IL-15 (10 ng/ml). IL-2 (20 Um′) and IL-4 (50 ng/ml). Phosphorothioate CpG oligodeoxynucleotide 2006 (Sigma), a TLR-9 agonist, and/or histidine tagged soluble recombinant human CD40L (50 ng/ml) and anti-poly-histidine mAb (5 μg/ml) (R&D Systems) were added at culture. CD40L was replaced by 3.75×10/ml mitomycin-treated CD40L transfectant.

Step 2. After 3 days of culture, a 3.7-fold cell expansion with ≥80% viable cells could be found if cells were cultured in step 1 with sCD40L and ODN. At day 4 of culture, the cells were harvested, washed and seeded at 2.5×10/ml with various combinations of cytokines: IL-2 (20 U/ml), IL-6 (50 ng/ml), IL-10 (50 ng/ml), and IL-12 (2 ng/ml) or IL-2 (20 U/ml), IL-6 (50 ng/ml), IL-10 (50 ng/ml) and IL-15 (10 ng/ml).

Step 3. To avoid the rapid cell death occurring after 3 days in step 2, cells were washed and cultured with IL-6+IL-15+IFN-α for 3 days. 60% of the cells died at this stage.

At day 7 of culture, cells were washed and cultured with IL-6 (50 ng/ml), IL-15 (10 ng/ml) and IFN-α (500 U/ml) for 3 days. In some cultures, HGF (20 ng/ml) and/or HA (100 μg/ml) were also added.

Immunoglobulin (Ig) production was measured in culture supernatants harvested at the end of each culture step: day 4, day 7 and day 10. IgM, IgA and IgG levels were evaluated by nephelometry with an automated Behring Nephelometer analyser II (Siemens, Paris, France). The sensitivity of the assay was 2 μg/ml for IgM, 3 μg/ml for IgA and 4 μg/ml for IgG. Ig production (pg/cell/day) was estimated dividing Ig amount in the culture supernatant by the number of living cells and the duration of the culture period. In agreement with detection of cytoplasmic Igs and expression of PC markers by flow cytometry, the rate of IgG production/cell/day increased 8 fold at day 10 compared to day 4. The final step consists of removing cytokines inducing proliferation (IL-2 and IL-10), and adding IFN-α, IL-6 and IL-15 yielding to PCs that express syndecan-1 and secrete higher amounts of Igs, as measured in the culture supernatants.

Adding IL-21 and/or APRIL did not result also in improvement of PC generation and survival and these in vitro generated PCs progressively died in culture.

As modified herein, for providing PBMCs->CD19+CD27+->Plasmablasts->Mature Plasma cells, sort CD19+CD27+1.5×105 cells/mL in 5 ml into wells of a 6 well plate.

### CD19+CD27+ B Cells Activated B Cells (Days 0-4)

IMDM

10% heat inactivated FBS (HIFBS: Invitrogen)

50 ug/mL human transferrin (Sigma)

5 ug/mL human insulin (Sigma)

20 U/ml IL-2 (Roche)

50 ng/ml IL-10

10 ng/ml IL-15

10 ug/ml phosphorothioate CpG Oligodeoxynucleotide 2006 (ODN, Sigma).

50 ng/mL histidine tagged soluble recombinant soluble recombinant human

CD40L.

### Activated B Cells→Plasmablasts (Days 4-7)

IMDM

10% heat inactivated FBS (HIFBS: Invitrogen)

50 ug/mL human transferrin (Sigma)

5 ug/mL human insulin (Sigma)

20 U/ml IL-2 (Roche)

50 ng/ml IL-10

10 ng/ml IL-15

50 ng/ml IL-6

### Plasmablasts→Plasma Cells (Days 7-10)

IMDM

10% heat inactivated FBS (HIFBS: Invitrogen)

50 ug/mL human transferrin(Sigma)

5 ug/mL human insulin (Sigma)

500 U/ml IFN-a

10 ng/ml IL-15

50 ng/ml IL-6

### Outputs:

### 1. Flow Cytometry: CD19, CD20, CD27, CD30, CD32b, CD38, CD138, HLA-DR

### 2. ELISA: IgM and IgG

### Consideration:

1. Include the following antibodies/stain: CD3, IgD, Live/Dead, CD24.
2. Replace ODN (TLR9 agonist) with candidate drug/therapy.
3. Plasma cells were identified as CD20−CD38+CD138+.
Exemplary comparative conditions: No gel, Growth Factor Reduced Matrigel (4.75 mg/mL), Rat Tail Collagen I (4.75 mg/mL, 1 mg/mL). Readouts

Brightfield and fluorescent (NucBlue) imaging performed at all timepoints.

Supernatant for IgG ELISA is collected at all timepoints.

Flow cytometry performed at termination.

Fluorescent imaging (NucBlue) on D0, D4, D7, D10.

Supernatant is collected for ELISAs on D0, D4, D7, D10.

Flow cytometry on D10.

Exemplary results: Differentiation in Matrigel resulted in a plasma cell population (CD138+CD38+CD20−). Differentiation in collagen did not result in a plasma cell population. CD27+ is not upregulated, suggesting B cells were not activated in this hydrogel. B Cells embedded in Matrigel resulted in IgG production. B Cells embedded in collagen did not produce IgG.

### Adaptations:

Scale down from 6 well plate.

Live/dead stain.

Normalization beads—Absence of the agonist leads to significant cell death.

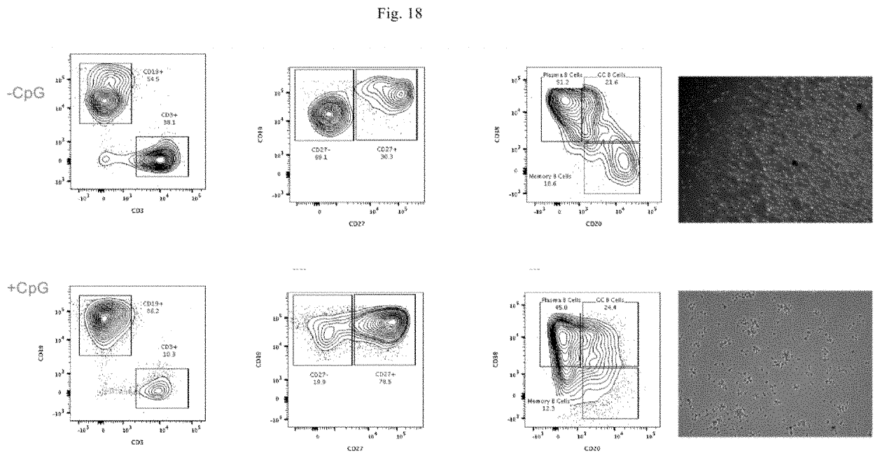

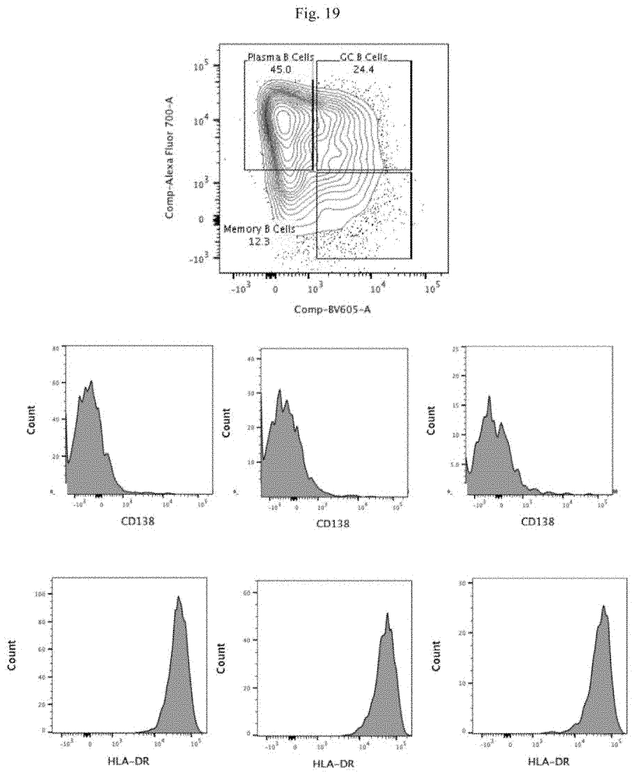

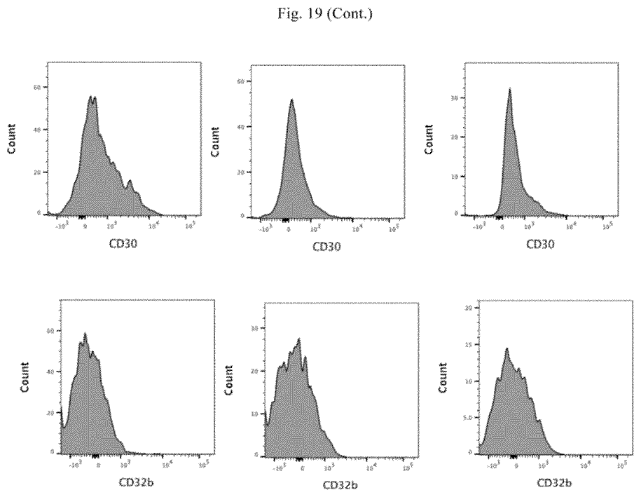

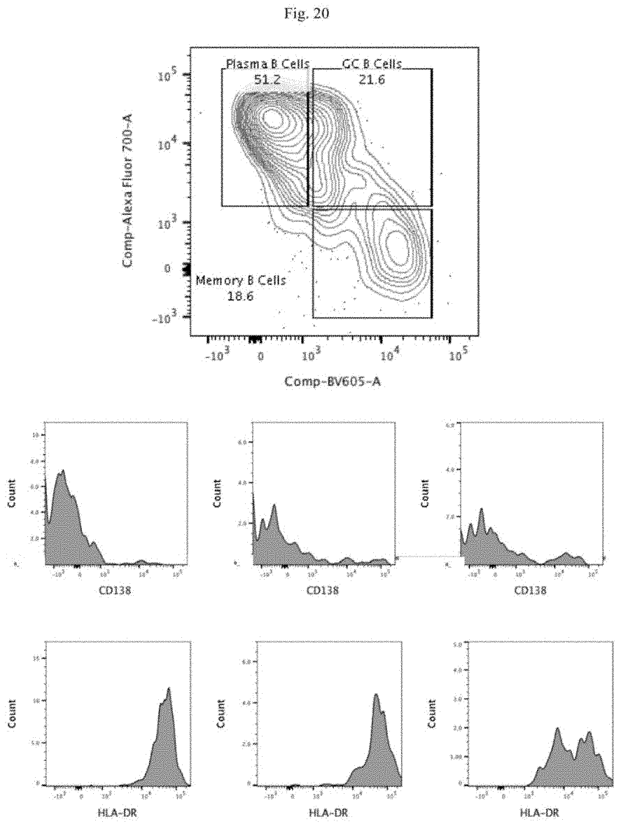

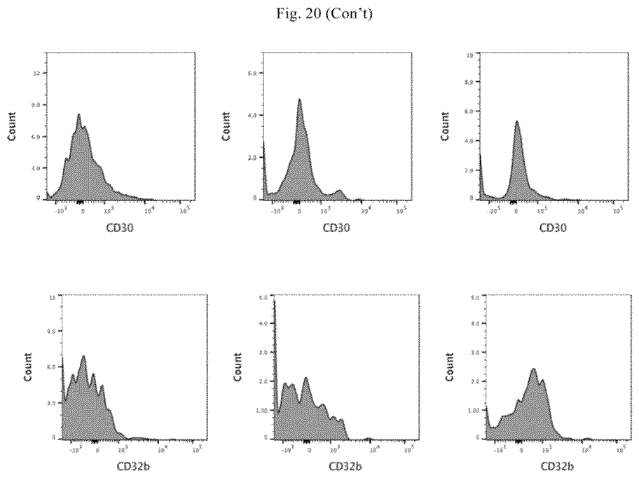

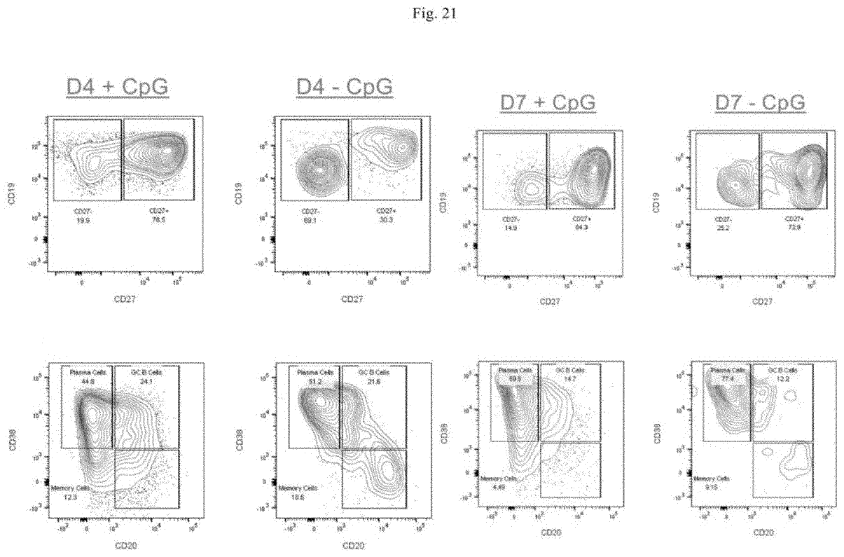

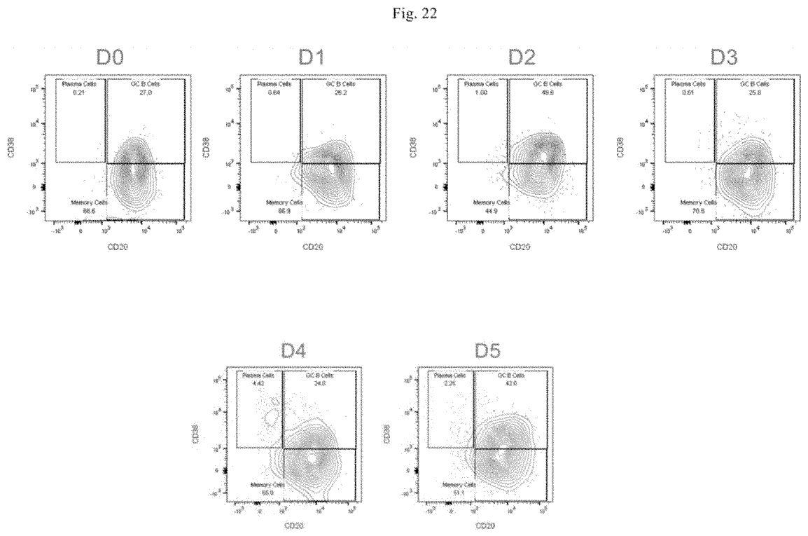

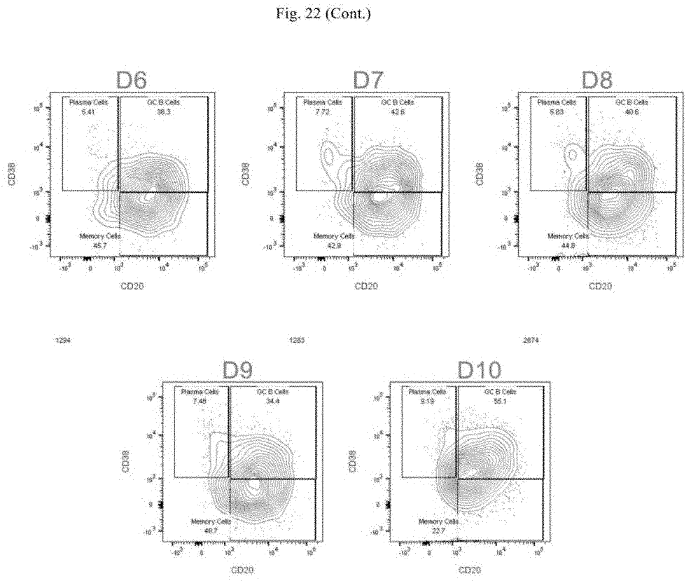

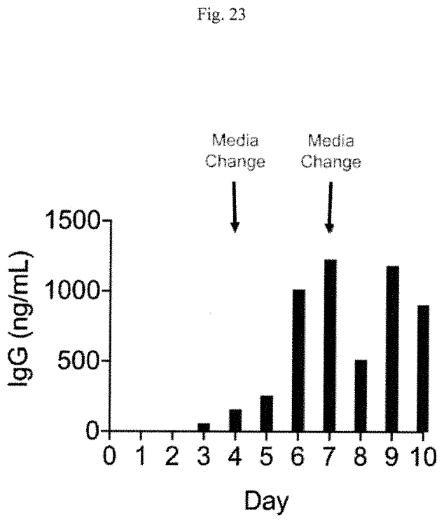

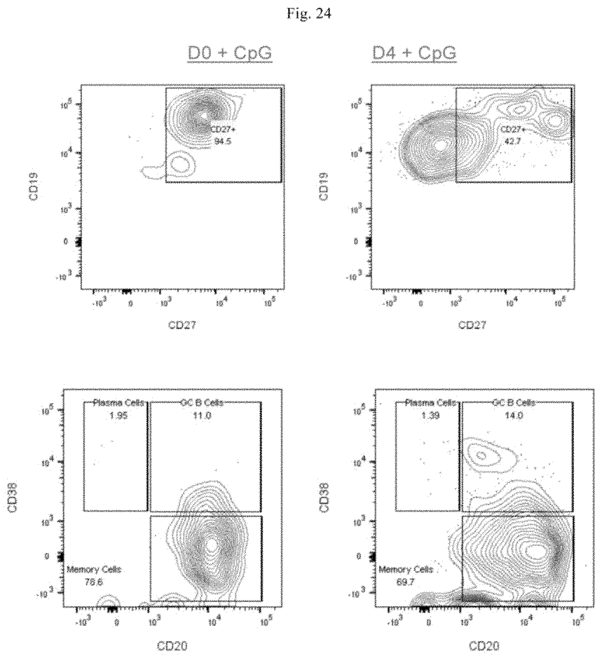

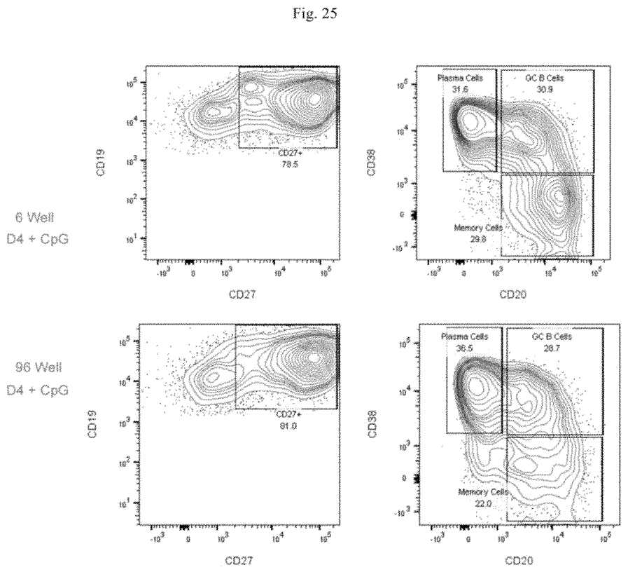

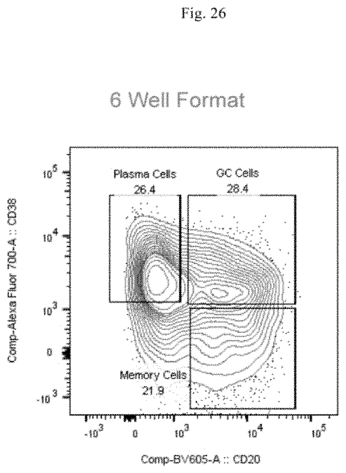

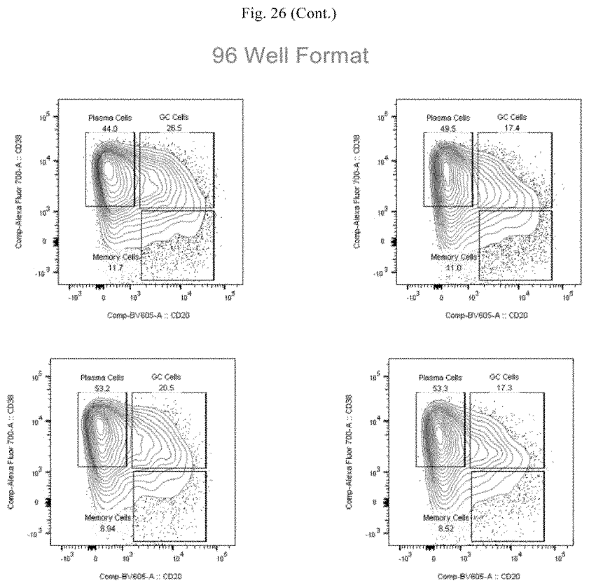

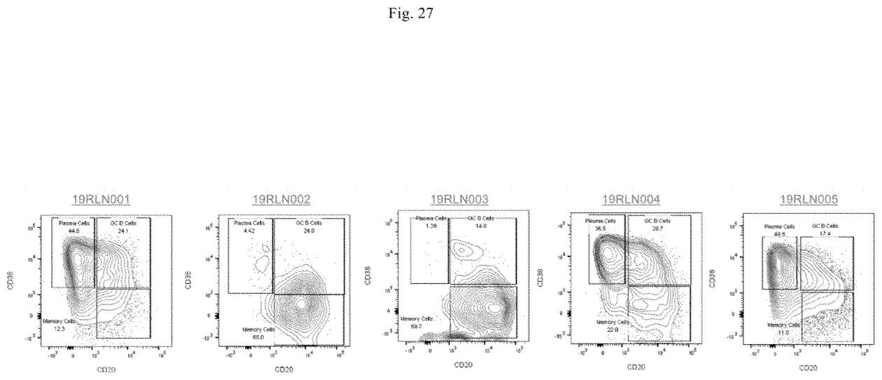

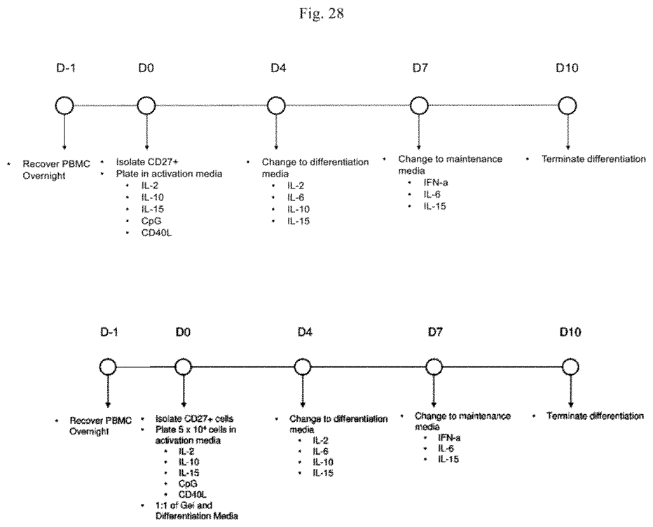

FIG. 18 shows an exemplary workflow for compositions and methods using soluble recombinant human CD40L.
FIG. 19 shows exemplary flow cytometry staining of CD19+CD27+ B cells, isolated from PBMCs, stimulated in the presence of a bacterial CpG DNA repeat segment (lower panels) and histidine tagged soluble recombinant human CD40L in a stimulation medium with soluble recombinant human CD40L, using a 3 step, 3 media differentiation method over 10 days, in 6 well plates. Upper panels show duplicate cultures without CpG DNA, right bright field image shows single cells while the lower bright field image shows numerous small cellular clusters in the presence of CpG DNA antigen.
FIG. 20 shows exemplary flow cytometry staining of cells shown in the previous figure without CpG DNA gated into plasma cells, germinal center cells and memory cells for comparing CD138, HLA-DR, CD30 and CD32b cell surface expression levels. Germinal center cells are shown in the middle column.
FIG. 21 shows exemplary flow cytometry staining of cells with CpG DNA gated into plasma cells, germinal center cells and memory cells for comparing CD138, HLA-DR, CD30 and CD32b cell surface expression levels. Germinal center cells are shown in the middle column.
FIG. 22 shows exemplary flow cytometry staining of cells without CpG DNA left panels and with CpG DNA right panels of Day 4 and 7, gated into plasma cells, germinal center cells and memory cells.
FIG. 23 shows exemplary flow cytometry staining of B cells over time in 96 well plates using soluble recombinant human CD40L, from CD19+ B cells showing a percentage of the memory cells producing germinal center cells then plasma cells.
FIG. 24 shows exemplary IgG production over time from CD19+CD27+ B cells using soluble recombinant human CD40L in 96 well plates.
FIG. 25 shows exemplary flow cytometry staining of B cells on Day 4 in 96 well plates from CD19+CD27+ B cells using soluble recombinant human CD40L, from CD19+ B cells showing a percentage of the memory cells producing germinal center cells then plasma cells at Day 0 and Day 4, with CpG DNA.
FIG. 26 shows exemplary flow cytometry staining of B cells on Day 4 in 96 well plates from CD19+CD27+ B cells using soluble recombinant human CD40L from CD19+ B cells showing a percentage of the memory cells producing germinal center cells then plasma cells at Day 0 and Day 4, with CpG DNA.
FIG. 27 shows exemplary flow cytometry staining of B cells on Day 4 in 6 well plants and 96 well plates from CD19+CD27+ B cells using the method modified using soluble recombinant human CD40L from CD19+ B cells showing a percentage of the memory cells producing germinal center cells then plasma cells at Day 0 and Day 4, with and without a media change.
FIG. 28 shows an exemplary flow cytometry summary of dot plots demonstrating staining of B cells over time comparing 6 well plates and 96 well plates from seeded B cells using soluble recombinant human CD40L for CD19+ B cells showing a percentage of the memory cells, germinal center cells and plasma cells.
In one embodiment, Collected 10,000 normalization beads, Gated on live cells.
In one embodiment, Recombinant Differentiation Timeline: Jourdan Approach, using soluble recombinant human CD40L.

B cells are the only cell type in this approach and all differentiation factors are supplemented to the media.

Recombinant CD40L is added to the activation media.

Flow cytometry is performed on D0, D4, D7, D10.

Supernatant is collected for ELISAs on D0, D4, D7, D10.

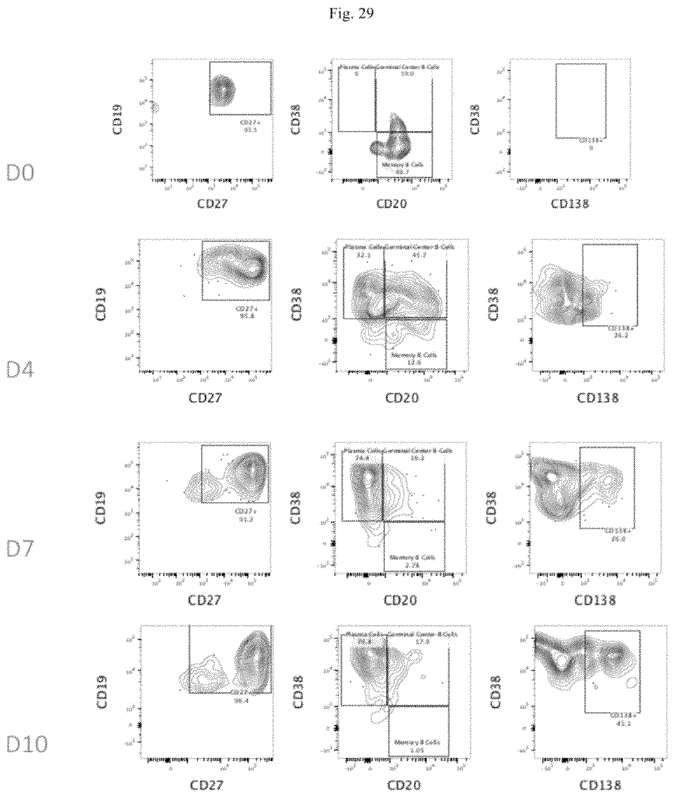

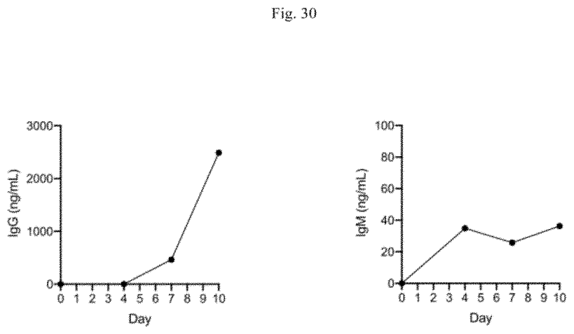

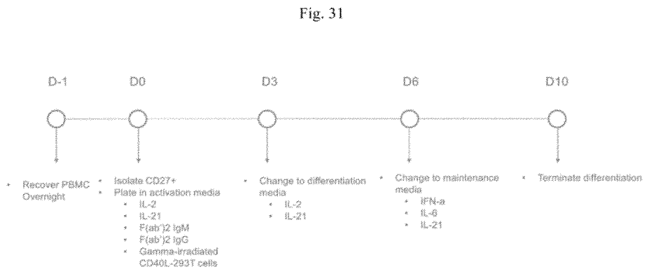

FIG. 29 shows an exemplary Workflow for compositions and methods using soluble recombinant human CD40L.
FIG. 30 shows exemplary flow cytometry staining of CD19+CD27+ B cells, isolated from PBMCs, stimulated in the presence of a bacterial CpG DNA repeat segment (lower panels) and histidine tagged soluble recombinant human CD40L in a stimulation medium with soluble recombinant human CD40L, using a 3 step, 3 media differentiation method over 10 days, in microfluidic chips.
FIG. 31 shows exemplary IgG and IgM production over time from CD19+CD27+ B cells using soluble recombinant human CD40L in microfluidic chips. IgG amounts are greater than previously shown from plate experiments.

4. Exemplary Compositions and Methods Tested Herein Producing Antibodies in Microfluidic Device Effluent Using CD40L Feeder Cells.

In some embodiments, IgG antibody production was measured after activation and differentiation using modified methods, specifically using CD40L as CD40L expressing feeder cell line, e.g. 293T cells. See, Cocco, et al., “In Vitro Generation of Long-lived Human Plasma Cells.” J Immunol. 189:5773-5785; Prepublished online 2012.

As described in Cocco, et al., mononuclear cells were isolated by Lymphoprep (Axis Shield) density gradient centrifugation. Total B cells were isolated by negative selection with the memory B cell isolation kit (Miltenyi Biotec). Twenty-four-well flat-bottom culture plates (Corning) and IMDM supplemented with Glutamax and 10% heat-inactivated FBS (HIFBS; Invitrogen) were used unless otherwise specified.

Days 0-3. B cells were cultured at 2.5 3 105/m1 with IL-2 (20 U/ml), IL-21 (50 ng/ml), F(ab 9) 2 goat anti-human IgM and IgG (10 m g/ml) on gamma-irradiated CD40L-expressing L cells (6.25 3 104/well).

Days 3-6. At day 3, cells were detached from the CD40L L cell layer and reseeded at 1 3 105/ml in media supplemented with IL-2 (20 U/ml), IL-21 (50 ng/ml), HybridoMax hybridoma growth supplement (11 m l/m1), Lipid.

Mixture 1, chemically defined and MEM amino acids solution (both at 1 3 final concentration).

Day 6 onward. At day 6, cells were reseeded at 2.5-5 3 105/ml in media supplemented with IL-6 (10 ng/ml), IL-21 (50 ng/ml), IFN-a (100 U/ml), HybridoMax hybridoma growth supplement (11 m l/m1), Lipid Mixture 1, chemically defined and MEM amino acid solution on gamma-irradiated M2-10B4 cells (4.16 3 104/well).

For transwell experiments, day 6 plasmablasts were seeded into the upper chamber of a 24-well plate transwell (clear polyester membrane, 0.4-m m pore; Corning) with the cytokines and media conditions described earlier. The lower chamber was left unseeded or seeded with gamma-irradiated M2-10B4 cells (4.16 3 104/well). Plasmablasts were also seeded in parallel in direct contact with M2-10B4 as described earlier.

For extended life-span maintenance, day 6 cells were harvested and recultured, either at 1.5 3 105 cells/well (total volume 1.5 ml) or at 3×105 cells/well (total volume 2.1 ml), in the upper compartment of a 24- or a 12-well plate transwell (clear polyester membrane, 0.4-mm pore; Corning), respectively. Gamma-Irradiated M2-10B4 stromal cells were seeded into the lower chamber at 4.16 3 104/well or at 8.32 3 104/well, proportionally. Cells were grown in IMDM supplemented with IL-6 (10 ng/ml), IL-21 (50 ng/ml), IFN-a (100 U/ml), HybridoMax hybridoma growth supplement (11 m l/m1), Lipid Mixture 1, chemically defined and MEM amino acids solution. IL-21 was discontinued after day 13. Every 3.5 d, a half (24-well) or a third (12-well) of the media volume was exchanged.

### Example G

As used herein, CD27+ B Cells→Plasmablast (Days 0-3)
IMDM
Glutamax
10% heat inactivated FBS (HIFBS: Invitrogen)
20 U/ml IL-2 (Roche)
50 ng/ml IL-21 (Peprotech)
10 ug/ml F(ab′)2 goat anti-human IgM and IgG (Jackson Immunoresarch).
Gamma-irradiated CD40L-expressing L cells (Use CD40L-293 instead).

### Plasmablast→Mature Plasma Cell (Days 3-6)

IMDM
Glutamax
10% heat inactivated FBS (HIFBS: Invitrogen)
20 U/ml IL-2 (Roche)
50 ng/ml IL-21 (Peprotech)
11 uL/m1 HybridoMax hybridoma growth supplement (Gentaur)
Lipid Mixture 1, chemically defined (final concentration at 1×) (200× stock, Sigma).
MEM amino acids solution (final concentration at 1×) (50× stock, Sigma) Mature Plasma Cell Maintenance (Day 6—onward).
IMDM
Glutamax
10% heat inactivated FBS (HIFBS: Invitrogen)
10 ng/ml IL-6 (Sigma)
50 ng/ml IL-21 (Peprotech)
100 U/ml IFN-α (Sigma)
11 uL/ml HybridoMax hybridoma growth supplement (Gentaur)
Lipid Mixture 1, chemically defined (final concentration at 1×) (200× stock, Sigma)
MEM amino acids solution (final concentration at 1×) (50× stock, Sigma)
Gamma-irradiated M2-10B4 cells

### Outputs: 1. Flow Cytometry: CD19, CD20, CD27, CD30, CD32b, CD38, CD138, HLA-DR. 2. ELISA: IgM and IgG

### Consideration:

1. Include the following antibodies/stain: CD3, IgD, Live/Dead, CD24.

2. Replace F(ab′)2 goat anti-human IgM and IgG with candidate drug/therapy.

3. Naïve B cells can be differentiated to plasmablasts but fail to generate long lived plasma cells.

4. BAFF and APRIL may need to be supplemented to support survival when using naïve B cells.

5. Plasma cells were identified as CD138 high CD38+.

Exemplary comparative conditions: No gel, Matrigel (4.75 mg/mL) and Collagen (4.75 mg/mL) and (1 mg/mL). Readouts. Fluorescent imaging (NucBlue) on D3, D6, D10. Supernatant is collected for antibody quantification ELISAs on D0, D3, D6, D10. Flow cytometry on D10.

Exemplary results: Differentiation in Matrigel and Collagen resulted in plasma cell populations (CD138+CD38+CD20-). B Cells embedded in Matrigel and collagen gels secrete IgG. No gel condition resulted in higher IgG production than hydrogel conditions.

Workflow for Differentiation supported by Feeder Cell Line: Cocco Approach, using a CD40L expressing feeder cell line, e.g. a gamma irradiated CD40L feeder cells.

Two cell type/line approach: B cells and CD40L expressing feeder cell line are co-cultured, initially.

Flow cytometry is performed on D0, D3, D6, D10.

Supernatant is collected for ELISAs on D0, D3, D6, D10.

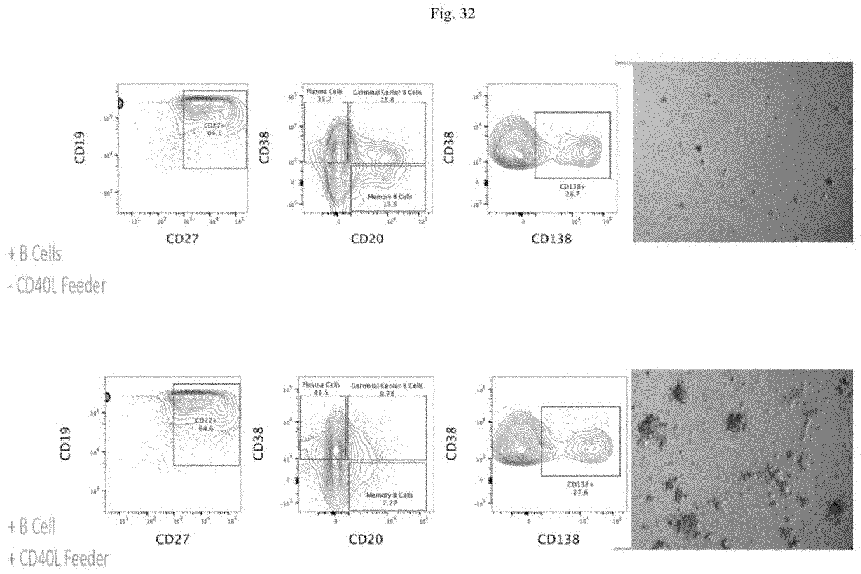

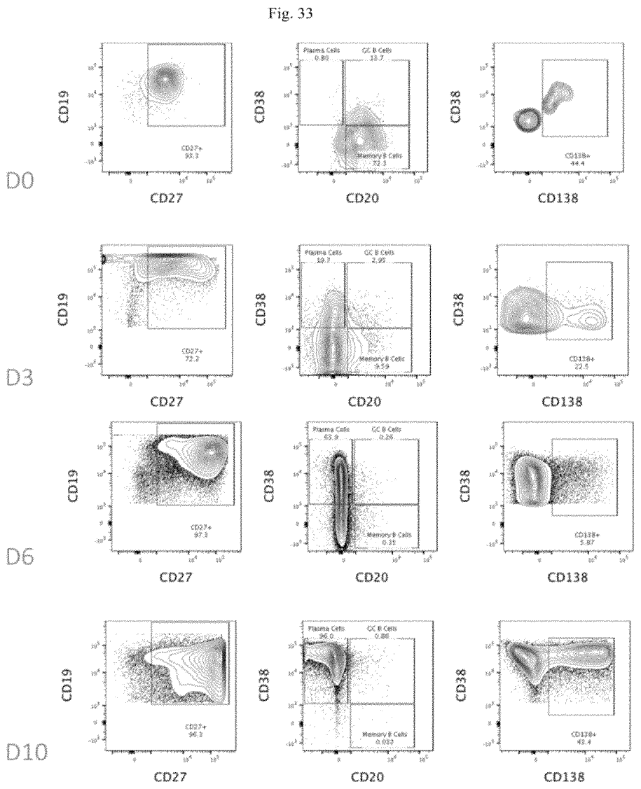

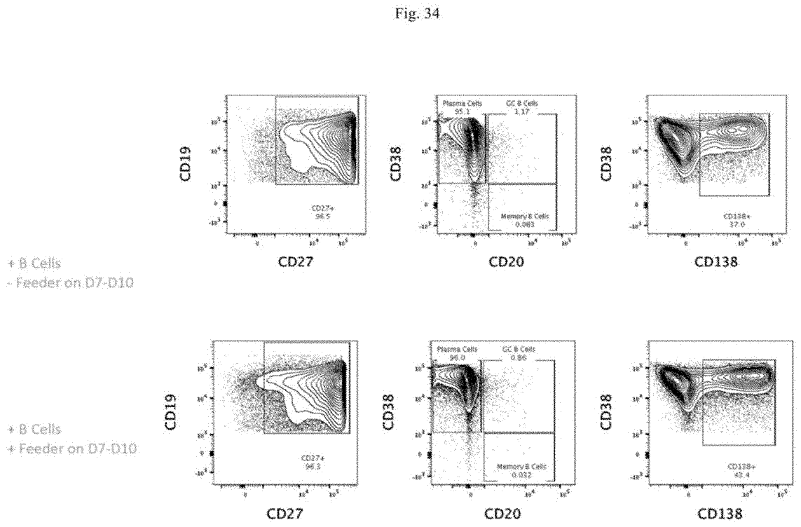

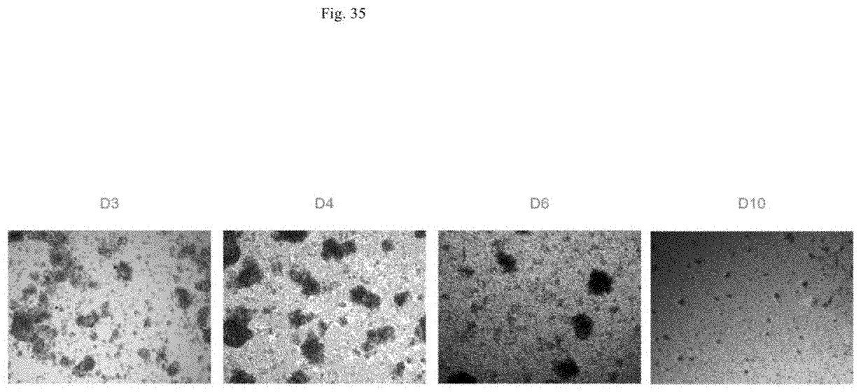

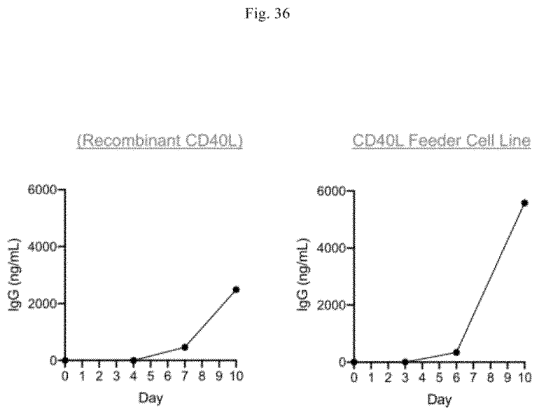

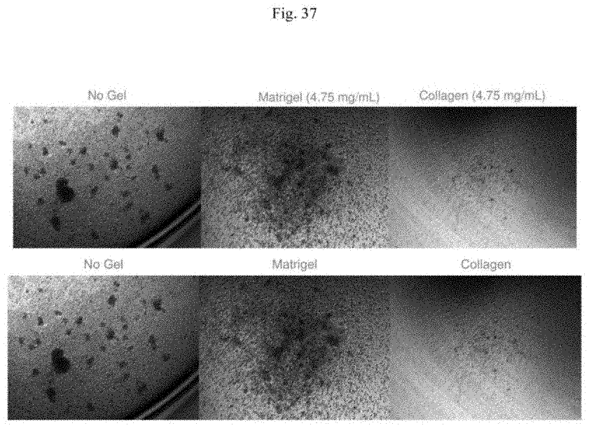

FIG. 32 shows an exemplary Workflow for compositions and methods modified from Cocco, et al. using gamma irradiated CD40L feeder cells.
FIG. 33 shows exemplary flow cytometry staining of CD19+CD27+ B cells, isolated from PBMCs, stimulated with or without the presence of CD40L expressing feeder cells in a stimulation medium with gamma irradiated CD40L feeder cells using a 3 step, 3 media differentiation method over 10 days, in 6 well plates. Upper panels show duplicate cultures without CD40L feeder cells, right bright field image shows single cells while the lower bright field image shows numerous slight and dark large cellular clusters.
FIG. 34 shows exemplary flow cytometry staining of CD19+CD27+ B cells, isolated from PBMCs, stimulated with or without the presence of gamma irradiated CD40L expressing 293T′ feeder cells in a stimulation medium of the present inventions but lacking soluble CD40L, using a 3 step, 3 media differentiation method over 10 days, in 6 well plates.
FIG. 35 shows exemplary day 10 flow cytometry staining of CD19+CD27+ B cells, isolated from PBMCs, stimulated with the presence of gamma irradiated CD40L expressing 293T feeder cells in a stimulation medium of the present inventions but lacking soluble CD40L, using a 3 step, 3 media differentiation method over 10 days, in 6 well plates. Lower panels, gamma irradiated CD40L expressing 293T feeder cells were added in the flow media from Day 7-Day 10.
FIG. 36 shows exemplary clustering of cells on Days 3-at least Day 6 compared to day 10 in 6 well plates using the method described in the previous figure including gamma irradiated CD40L expressing 293T feeder cells.
FIG. 37 shows exemplary IgG production over time from CD19+CD27+ B cells using the method using recombinant CD40L in microfluidic chips compared to IgG amounts using the method including gamma irradiated CD40L expressing feeder cells.

In one embodiment, Recombinant Differentiation Timeline: Jourdan Approach, using recombinant CD40L.

B Cells form clusters in all conditions based on brightfield microscopy.

Cell number is higher in the no gel and Matrigel conditions compared to collagen gels.

In hydrogels, cells aggregate at center of the gels in contrast to the no gel condition where cells cluster on the perimeter of the 96 well.

Imaging (NucBlue, Ethidium Homodimer 1 and Caspase 3/7) on D0, D4, D7, D10.

Supernatant is collected for ELISAs on D0, D4, D7, D10.

Flow cytometry not included until cell recovery from gels optimized.

Conditions: No gel, Matrigel and Collagen (4.75 mg/ml).

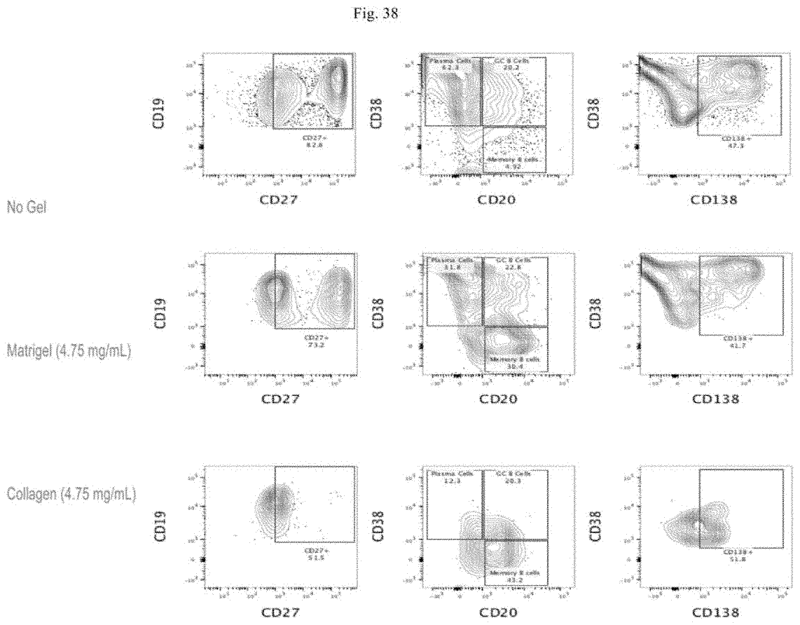

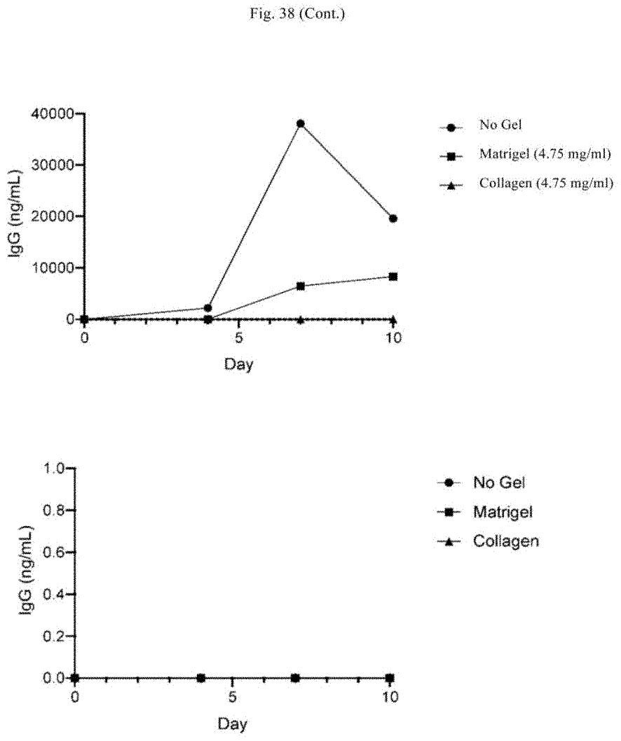

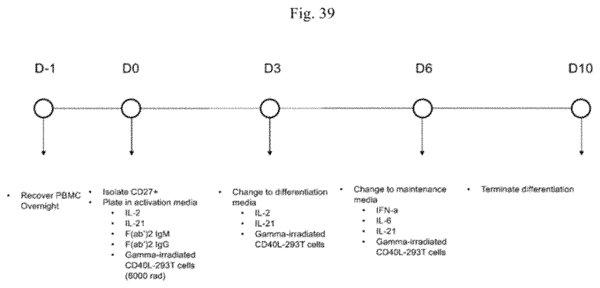

FIG. 38 shows exemplary bright field images on Day 10, method modified from Jourdan, et al. using recombinant CD40L in plates.
FIG. 39 shows exemplary flow cytometry and IgG production on Day 10, method using recombinant CD40L in plates.
D3 Treatment with Feeder Cell Line: Cocco Approach, using gamma irradiated CD40L expressing feeder cells.

Imaging (NucBlue, Ethidium Homodimer 1 and Caspase 3/7) on D3, D6, D10 Supernatant is collected for ELISAs on D0, D3, D6, D10.

Flow cytometry not included until cell recovery from gels optimized.

Conditions: No gel, Matrigel and Collagen (4.75 mg/mL) and (1 mg/mL).

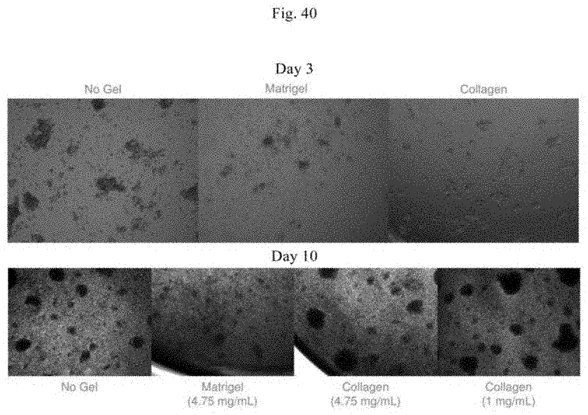

FIG. 40 shows an exemplary Workflow for compositions and methods using gamma irradiated CD40L feeder cells.

D10 Treatment with Feeder Cell Line: Cocco Approach, using gamma irradiated CD40L expressing feeder cells. — Brightfield Microscopy—D10.

B Cells formed 3D structures in all conditions.

More clusters formed in collagen gels (4.5 mg/mL and 1 mg/mL) than Matrigel.

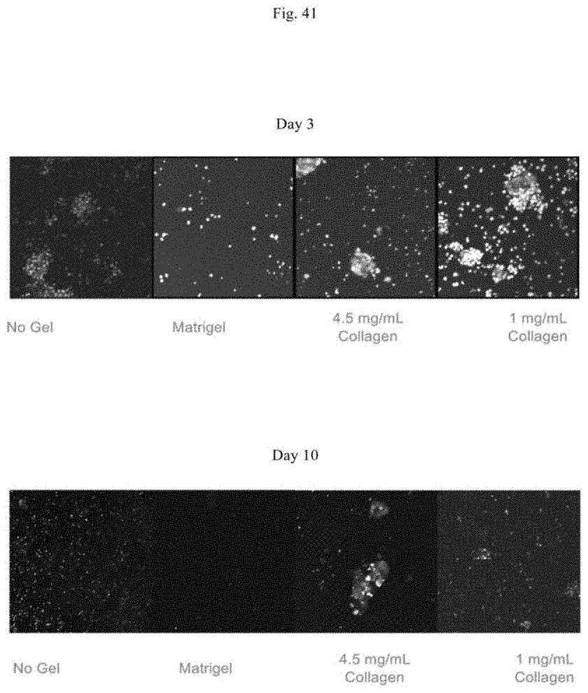

FIG. 41 shows exemplary clustering of cells on Day 3 and Day 10 comparing clustering in 6 well plates using the modified method including gamma irradiated CD40L expressing 293T feeder cells of cells in plates coated with no gel, Matrigel and two different amounts of collagen.
Differentiation with Feeder Cell Line: Cocco Approach D3

### Fluorescence Microscopy D3

Confocal microscopy of nuclear staining confirmed that clusters of B cells were seen in collagen and no gel conditions, but not in Matrigel.

In the collagen gels, these clusters were present at various depths within the gels.

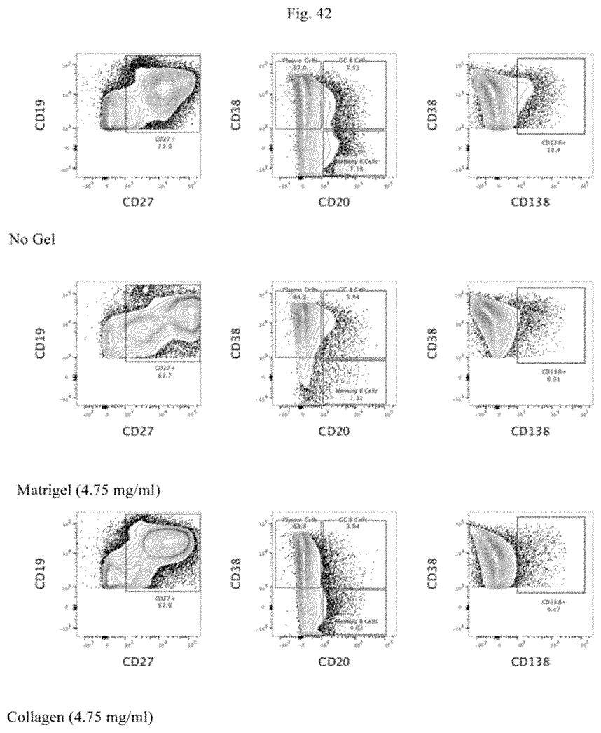

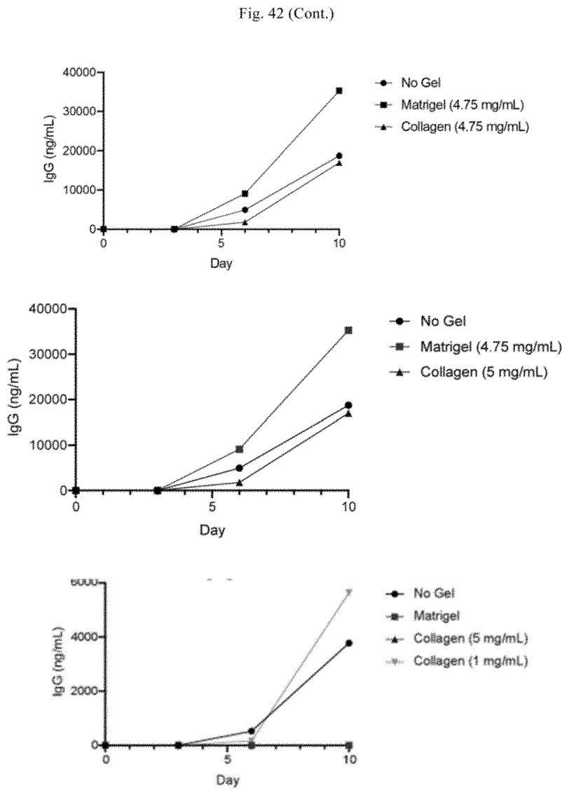

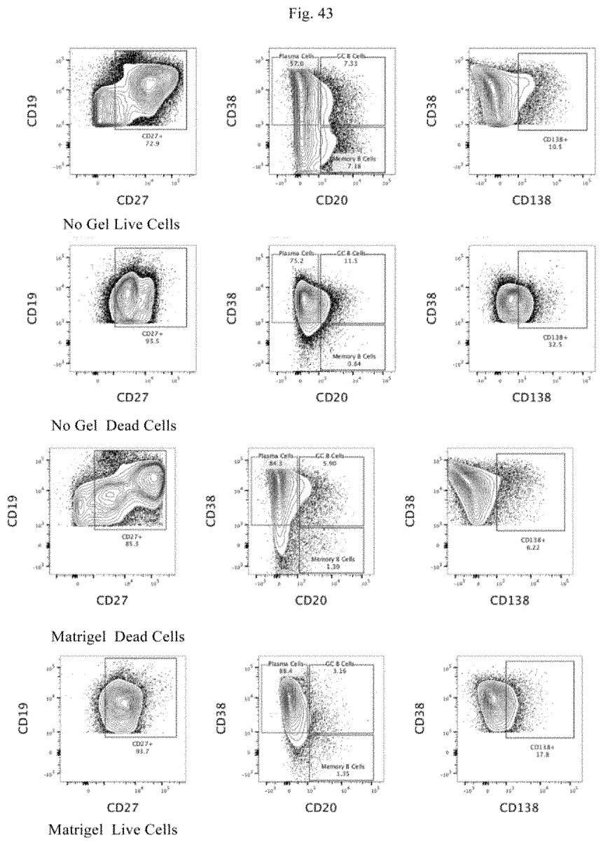

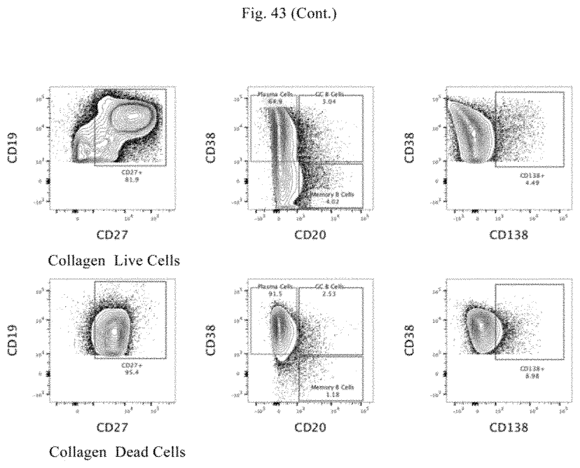

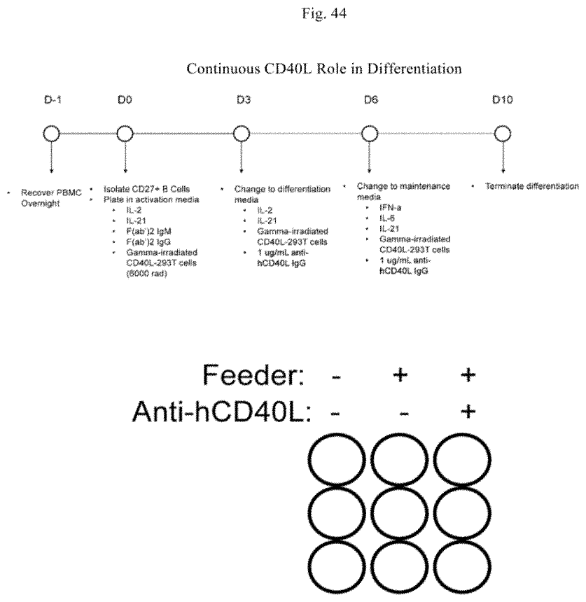

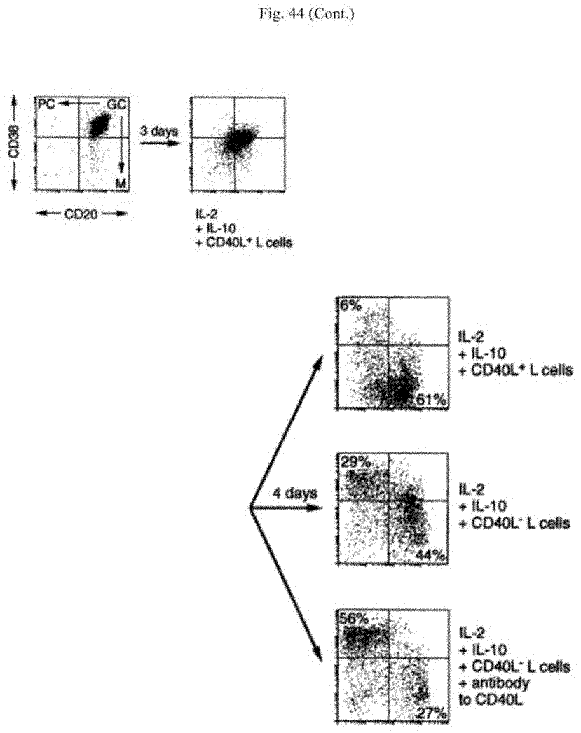

FIG. 42 shows exemplary clustering of cells on Day 3 and Day 10 comparing clustering in 6 well plates using the modified method including gamma irradiated CD40L expressing 293T feeder cells of cells in plates coated with no gel, Matrigel and two different amounts of collagen.
FIG. 43 shows exemplary flow cytometry staining of live-dead cells using methods including gamma irradiated CD40L expressing 293T feeder cells of cells in plates coated with no gel, Matrigel and two different amounts of collagen. Exemplary comparisons of IgG production.
FIG. 44 shows exemplary flow cytometry staining of live-dead cells using methods including gamma irradiated CD40L expressing 293T feeder cells of cells in plates coated with no gel, Matrigel and two different amounts of collagen.
Exemplary comparisons of activated cells, germinal center cells and plasma cells.

### Aim:

Determine whether inhibiting CD40L after activation enhances plasma cell differentiation and Ig production in 3D.

### Significance:

Determine whether a continuous T and B cell co-culture in a gel will yield optimal Ig production.

### Outputs:

Supernatant is collected for ELISAs on D0, D3, D6, and D10.

Brightfield images are taken on D0, D3, D6, and D10.

Flow cytometry on D10.

Gel Conditions: 4.75 mg/mL Matrigel and 1 mg/mL Collagen I.

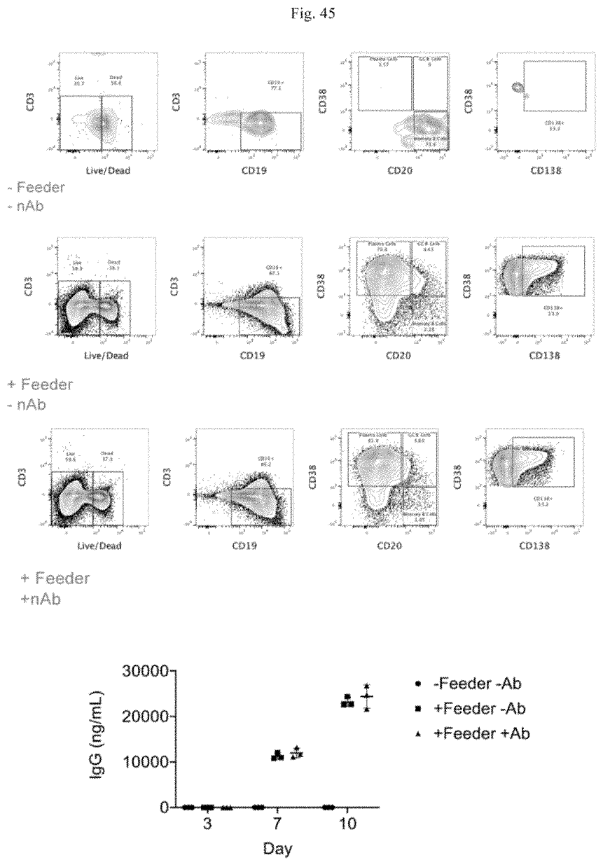

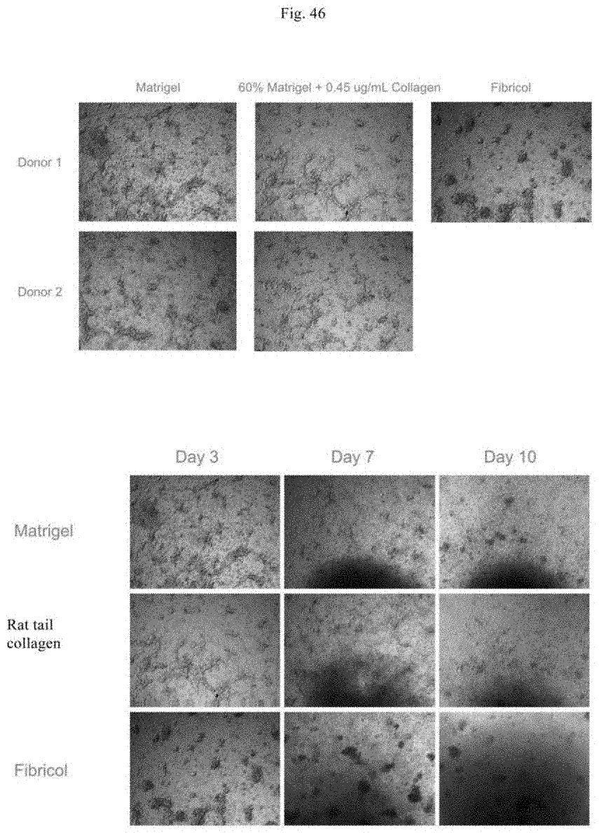

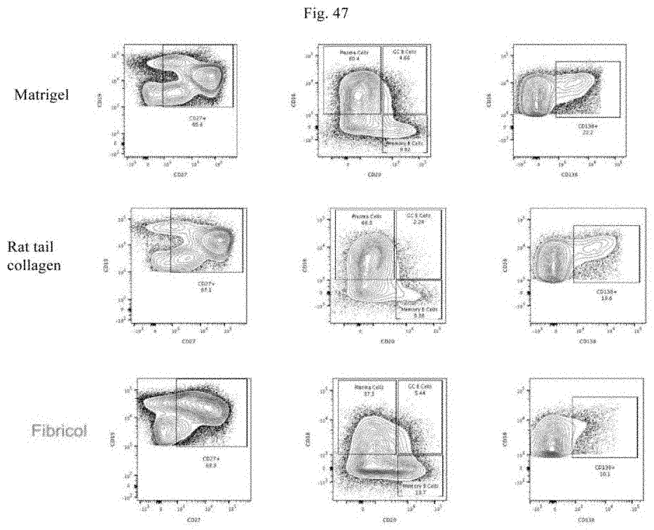

FIG. 45 shows one exemplary embodiment—workflow for methods including gamma irradiated CD40L expressing feeder cells along with exemplary flow cytometry staining of CD19+CD27+ cells, stimulated with or without the presence of CD40L expressing feeder cells in a stimulation medium including CD40L, IL-2, IL10, for 3 days followed by 4 days in either IL-2, IL-10, CD40L with or without CD40L feeder cells, and lower dot plot with CD40L and antibody blocking CD40L without CD40L feeder cells.
FIG. 46 shows exemplary flow cytometry staining of CD19+CD27+ cells, as in the previous figure.
In one embodiment, Donor 1 Results:
Clustering of B cells around CD40L is most pronounced in Fibricol.
Cells cluster on top of Matrigel while B cells in the rat collagen and Fibricol gels are spread.
   FIG. 47 shows exemplary clustering of cells on Days 3, 7 and 10 comparing clustering in 6 well plates using the modified method including gamma irradiated CD40L expressing 293T feeder cells of cells in plates coated with no gel, Matrigel and Fibricol. Darker clusters develop using Fibricol. Lower panels show exemplary IgG production over time from CD19+CD27+ B cells using the method with or without CD40L feeder cells and with or without blocking CD40L antibody.
   In one embodiment, Results:

Matrigel and rat tail collagen Gel results in similar frequencies of differentiated plasma cells.

Matrigel resulted in twice as many CD38+CD20−CD138+ cells.

Fibricol produced the lowest frequency of CD30+CD2—cells.

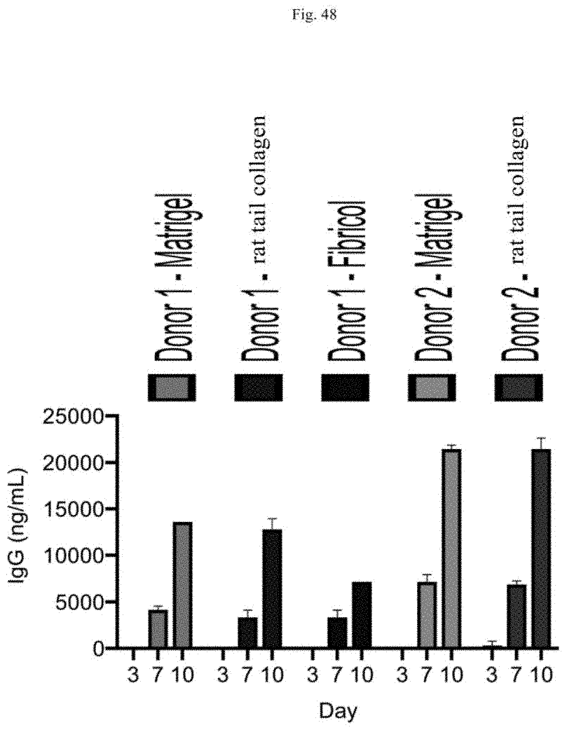

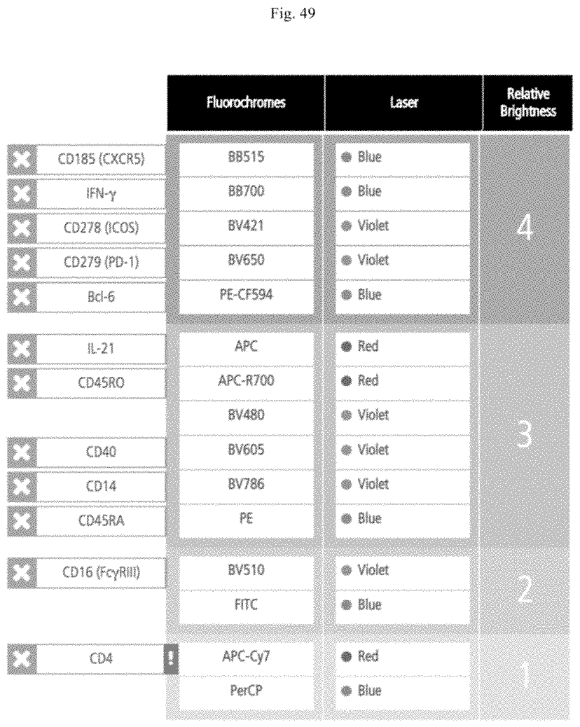

FIG. 48 shows exemplary flow cytometric analysis of cell populations using the method using CD40L feeder cells in the presence of hydrogels of Matrigel, rat tail collagen and bovine collagen, Fibricol, in 6 well plates comparing donors and over time.
FIG. 49 shows exemplary IgG production over time from CD19+CD27+ B cells using the method modified from Cocco, et al., using CD40L feeder cells in the presence of hydrogels of Matrigel, rat tail collagen and bovine collagen, Fibricol, in 6 well plates. IgG production is similar throughout the different gel composition. The main factor appears to be donor variability using this method.

5. Exemplary Embodiments of Microfluidic Devices as AB-Lymph Node Chips Using Follicular CD4+Helper Cells.

T follicular helper cells (Tfh) refer to a specialized subset of CD4+ T cells that were identified in the human tonsil white blood cells, and contemplated for providing the boost observed in white blood cell culture on chip providing longer life spans to B cells. In some embodiments, IgG antibody production was measured after activation and differentiation using modified methods, specifically using TLR8 agonists for producing follicular T helper cells. See, Ugolini, et al., “Recognition of microbial viability via TLR8 drives TFH cell differentiation and vaccine responses.” Nature Immunology 19(4), 386-396; 2018. Around 30 ng/ml highest IgG measurement shown.

Untouched human CD1+mDC were purified by negative selection via immunomagnetic bead separation (Miltenyi Biotec, Bergisch Gladbach, Germany) following the manufacturer's instructions. Naïve CD4+ T cells were purified by immunomagnetic separation using negative selection (MagniSort™ Human CD4 Naïve T cell Enrichment Kit, eBioscience, San Diego, Calif.).

Total CD4+ T cells, when used, were isolated by magnetic separation using negative selection (MagniSort™ Human CD4 T cell Enrichment Kit, eBioscience). Untouched naïve human B cells were isolated by immunomagnetic bead separation (Miltenyi Biotec, Bergisch Gladbach, Germany) following the manufacturer's instructions. Cell purity was routinely checked by flow cytometry and only purities of >85% (monocytes) and >95% (T and B) cells were used for subsequent experiments.

T cells were cultured in RPMI1640 supplemented with 10% human serum (from the respective T cell donor), 1% glutamine, 1% HEPES buffer, 1% non-essential amino acids, some T cell conditions were supplemented with 2.5 ng/ml of TGF-β (eBioscience, San Diego, Calif.). Cells were grown at 37° C., 5% CO 2 in a humidified incubator.

For monocyte: T cell co-cultures monocytes were cultured as described above and stimulated as indicated (e.g. EC, HKEC MOI 1-25) in antibiotic-free medium. After one and a half hours, penicillin/streptomycin (1%) was added together with autologous naïve CD4+ T cells at a monocyte to T cell ratio of 2:1 and staphylococcal enterotoxin B (SEB, Sigma) at a concentration of 1.0 μg/ml. After 5 days of co-culture T cells were harvested, washed, restimulated with Phorbol-12-myristat-13-acetat (PMA, 50 ng/ml) and Ionomycin (1 μg/ml, both obtained from Sigma), stained and analyzed by flow cytometry.

For T: B cell co-cultures, T cells were differentiated by co-cultures with autologous monocytes for 6 days as described before. CXCR5+ICOS+PD-1hi T cells were sorted by flow cytometry (BD FACS-Aria II) and added to naïve autologous B cells at a T to B cell ratio of 1:2 in the presence of SEB (1 μg/ml). After 12 days co-culture B and T cells were harvested and analyzed by flow cytometry. For analysis of plasma blast differentiation, sorted T FH (CD19−CD4+CD45RA−CXCR5+) or naive (CD19−CD4+CD45RA+) T cells were cocultured with memory B cells at a ratio of 1:1 in the presence of 4 ng/ml SEB for 7 days.

### Example H

### Using a Toll-Like Receptor 8 (TLR8).

Human monocytes, T cells and B cells used in this study were either freshly isolated from peripheral venous blood of healthy volunteers or from commercially obtained buffy coats.

As used herein, PBMC->APC+TLR8 Agonist->TFH->TFH+B Cells->Plasma cells.

### CD14+CD16− Monocyte Isolation (Day 0)

Density Gradient

CD16 negative selection using EasySep

Culture using RPMI 1640

10% Fetal calf serum (Sigma)

1% glutamine (Sigma)

1% HEPES buffer (Sigma)

1% non-essential amino acids (Sigma)

### T Cell Isolation

CD4+Naive T Cell Negative Selection (Magnetic separation, ebioscience)

Culture using RMPI 1640

10% human serum

1% glutamine

1% HEPES buffer

1% non-essential amino acids

Generating TFH Cells—Monocyte & CD4 Co-culture (Days 1-5)

Activate monocytes in antibiotic free media using TLR agonist, CL075 at 1 ug/mL.

After 1.5 hours, add naïve CD4 T Cells at a ratio of 2:1 monocytes/T cell in RPMI 1640.

10% human serum

1% glutamine

1% HEPES buffer

1% non-essential amino acids

1% Penicillin-streptomycin

1 ug/ml Staphylcoccal enterotoxin B (SEB, Sigma)

5 day co-culture

Generating CD27^{hi}CD38+Plasma Cells— T cell & B cell Co-culture (Day 5-11)

Sort TFH cells by flow cytometry (CXCR5+ICOS+PD-1^{hi}).

Incubate T cells/memory B cells at a ratio of 1:2 in RMPI 1640.

1 ug/ml Staphylococcal enterotoxin B (SEB, Sigma).

7 day co-culture

### Outputs:

### 1. Flow Cytometry: CD19, CD20, CD27, CD30, CD32b, CD38, CD138, HLA-DR

### 2. ELISA: IgM and IgG

### Consideration:

1. Include the following antibodies/stain: CD3, IgD, Live/Dead, CD24.
2. Replace SEB with candidate drug/therapy.
3. Plasma cells were identified as CD27 hi CD38+.

Generation of CD27+CD20−CD38+CD138 hi Plasma Cells (Flow).

Decreased production of IgM as differentiation progresses (ELISA).

Increased production of IgG as differentiation progresses (ELISA).

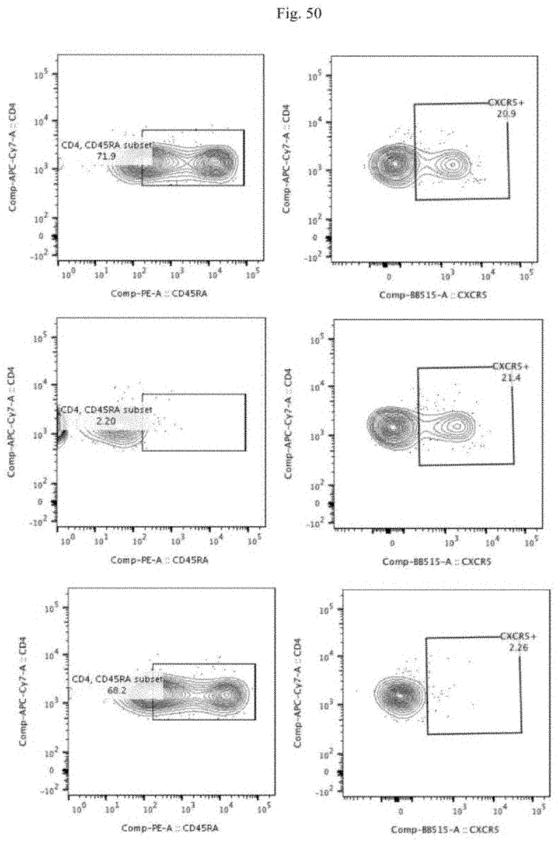

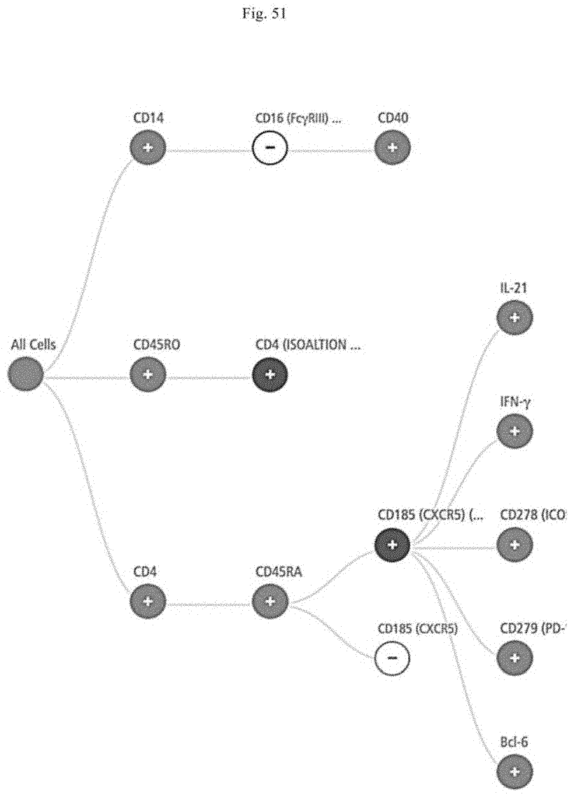

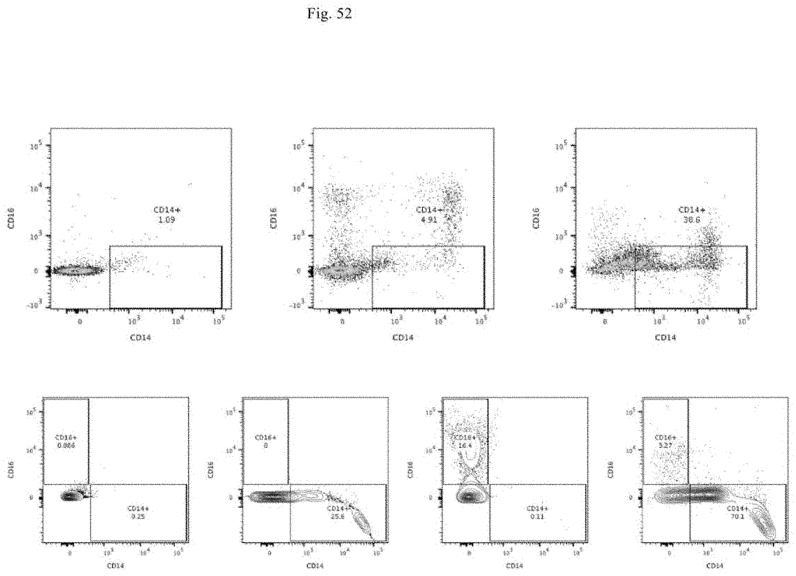

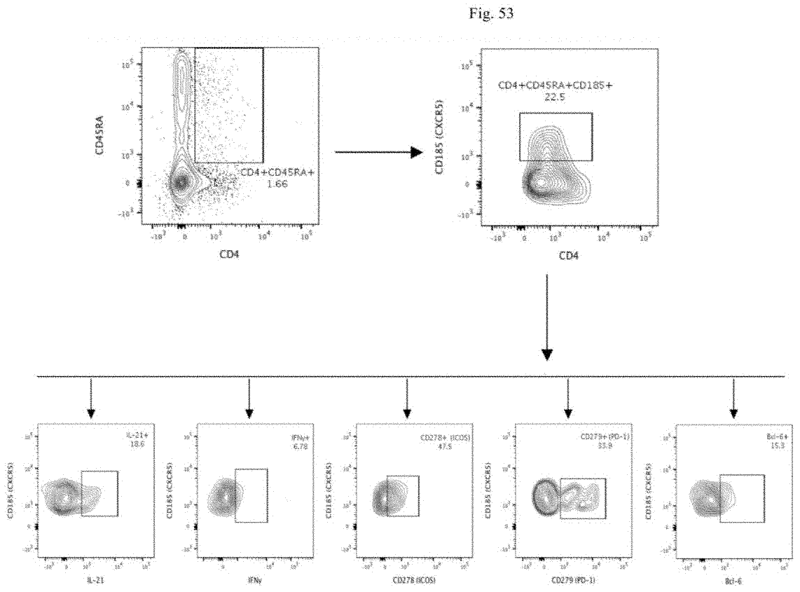

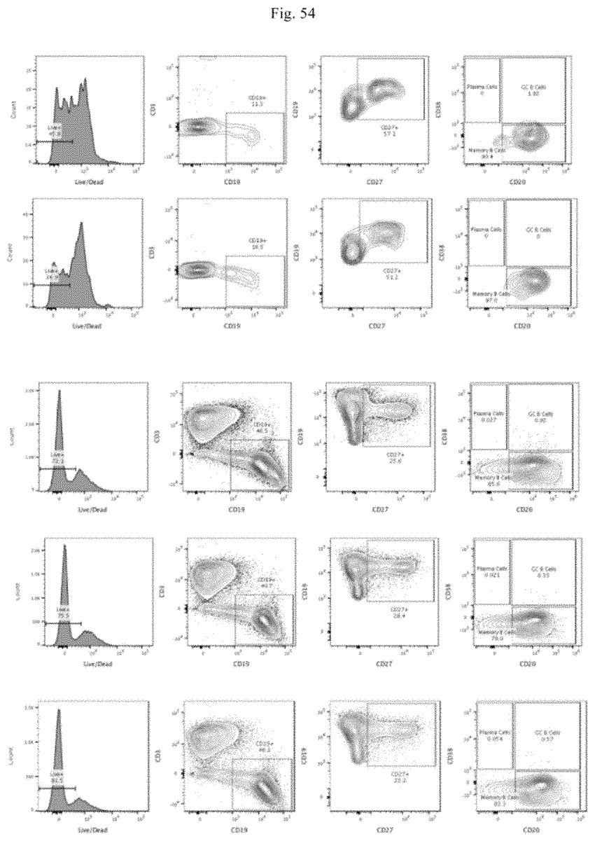

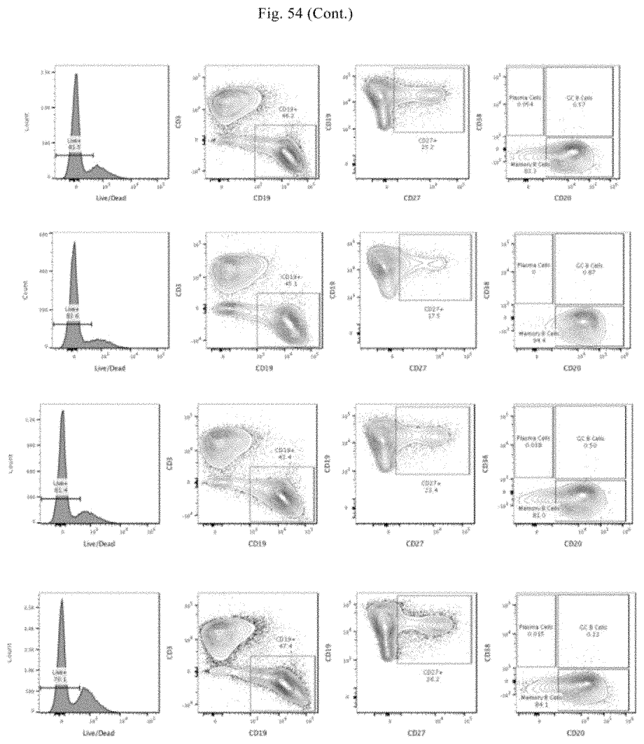

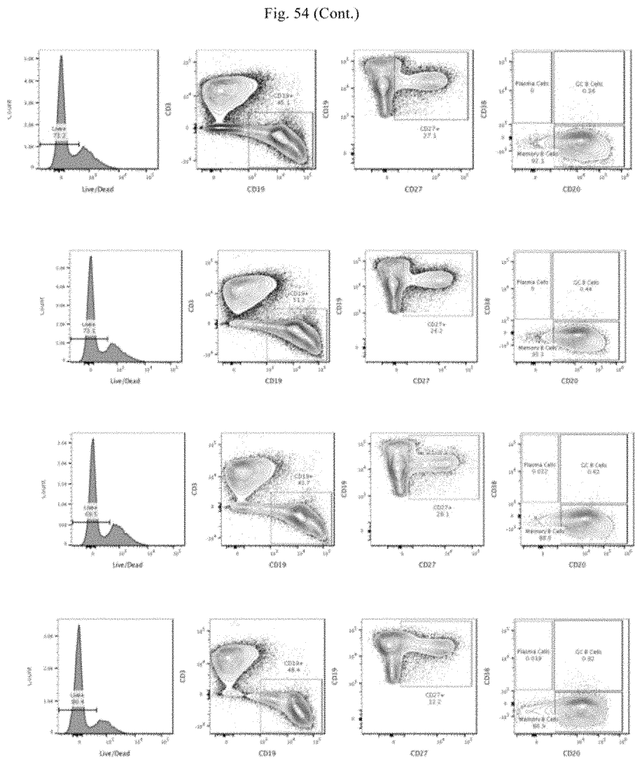

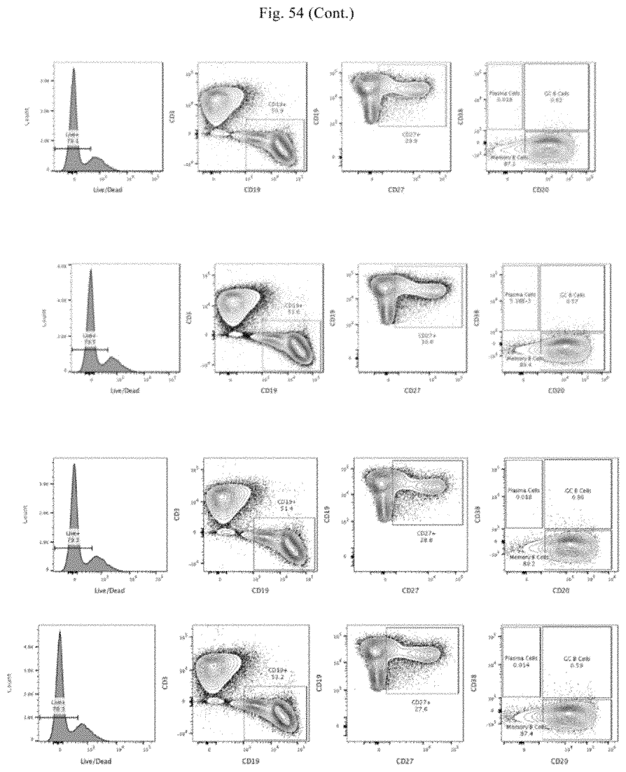

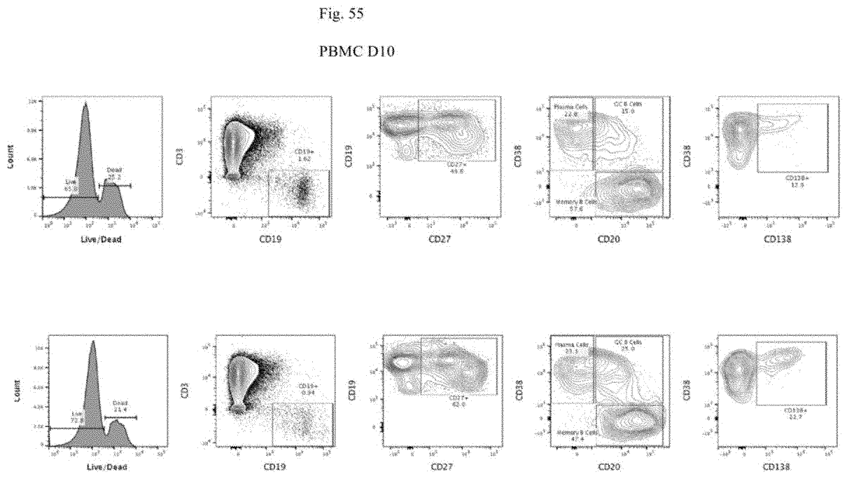

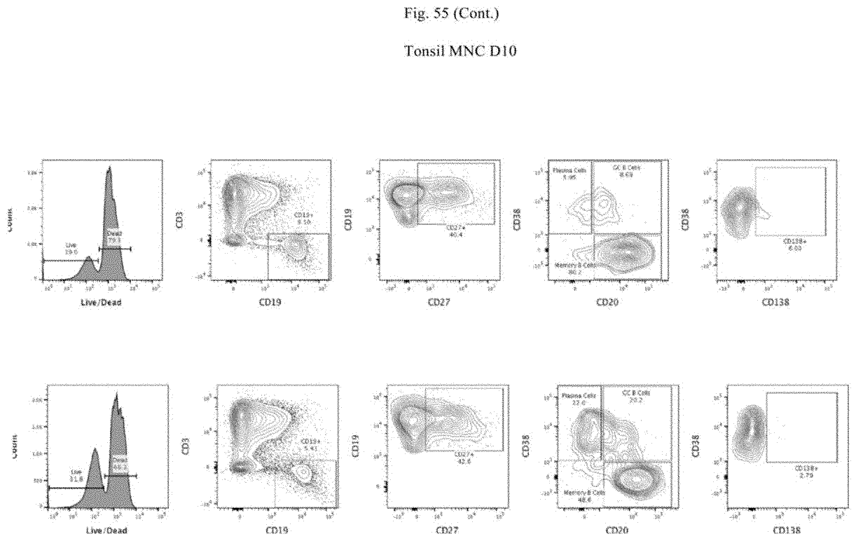

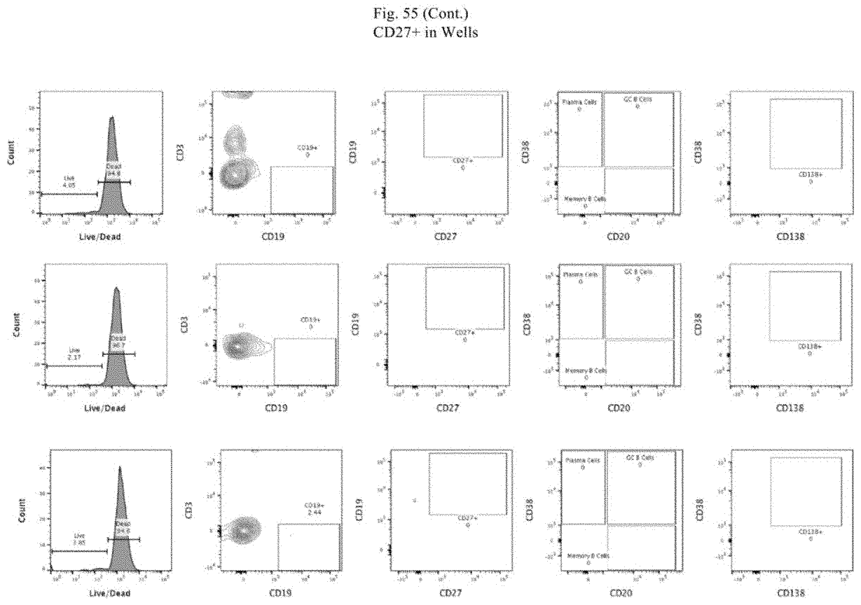

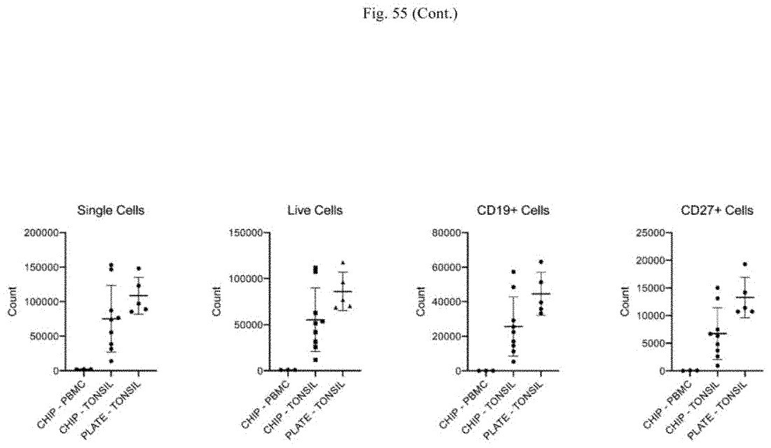

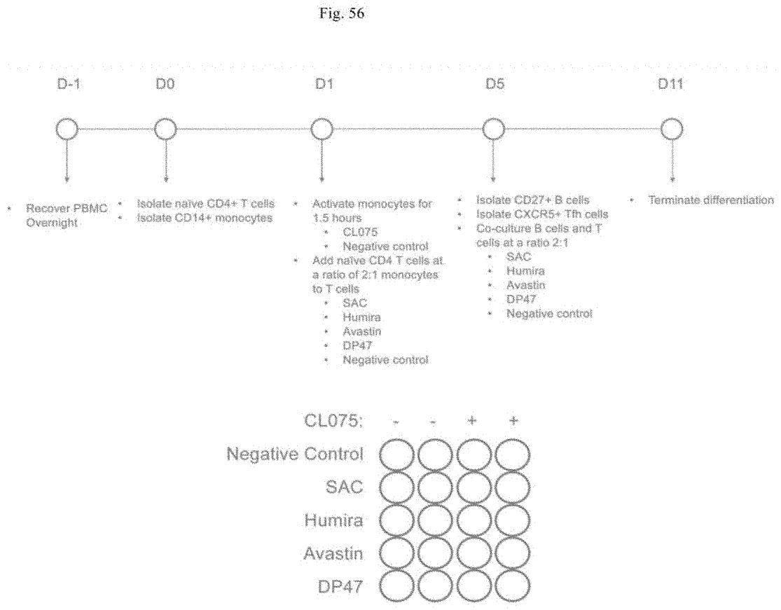

FIG. 50 shows exemplary biomarker panel for monocyte related biomarkers.
FIG. 51 shows exemplary flow cytometry staining for Continuation of Flow Cytometry Panel by FMO (Fluorescence Minus One Control, or FMO control) referring to a type of control used to properly interpret flow cytometry data. It is used to identify and gate cells in the context of data spread due to the multiple fluorochromes in a given panel.
FIG. 52 shows an illustration of an exemplary lineage of monocytes and CD4 cells by selected biomarker expression.
FIG. 53 shows exemplary flow cytometry staining of tonsil cells using monocyte and CD4 cell biomarkers from the previous figures.
FIG. 54 shows exemplary flow cytometry gating strategy for monocyte and CD4 cells from cultures.
FIG. 55 shows exemplary flow cytometry staining of PBMCs and tonsil cells obtained from multiple donors using CD40L feeder cells.
WBS in tonsil cell seeding show higher numbers of live cells, CD19+ and CD27+ cells.

2 million PBMC in S1

2 million Tonsil MNC (mononuclear cells) in S1

50,000 CD27+ B Cells in 96 Well

Gel: 60% Matrigel+0.45 mg/mL Fibricol (Bovine)

Media Changes on D3, D6, D10

No CD40L 293T Cells, recombinant mCD40L added to culture D0-D3.

FIG. 55 shows exemplary flow cytometry staining on Day 10 comparing embodiments of microfluidic chips seeded with WBCs in hydrogels from either PBMC D10 (Gated on Live Cells) or Tonsil MNC D10 (Gated on Live Cells) compared to the same methods but using CD19+CD27+ cells in Wells (Gated on Live Cells) using a 3 step procedure over 11 days including T follicular helper cells demonstrating more: cells, live cells, CD19+ cells and CD27+ cells in tonsil white blood cell populations.
In one embodiment, Ugolini Approach, including T follicular helper cells.

### Aim:

Determine whether Tfh cells can be generated in vivo using CL075 for use in B cell differentiation.

### Significance:

Evaluate the ability of Tfh cells to support plasma cell production and antibody production in vitro in comparison to naïve CD4 T cells.

### Outputs:

Flow cytometry on D5 to evaluate activation of monocytes and differentiation of naïve CD4 into Tfh cells.

In one embodiment, Workflow for including T follicular helper cells.

D-1•Recover PBMC Overnight.

D0•Isolate naïve CD4+ T cells•Isolate CD14+ monocytes.

D1 Activate monocytes for 1.5 hours.

CL075

Negative control

Add naïve CD4 T cells at a ratio of 2:1 monocytes to T cells.

### Examples of Test Substances•SAC•Humira•Avastin•DP47

Negative control

D5•Isolate CD27+ B cells•Isolate CXCR5+ Tfh cells.

Co-culture B cells and T cells at a ratio 2:1.

### Examples of Test Substances•SAC•Humira•Avastin•DP47

Negative control

D11•Terminate differentiation.

### Considerations

Using CD14+CD16+/− Isolation.

Switch to CD14+CD16− Isolation.

Measure upregulation of CD40L on monocytes post CL075 treatment.

Increase CL075 concentration (2-Propylthiazolo[4,5-c]quinolin-4-amine, a selective agonist of Toll-like receptor-8 (TLR-8) with immunostimulant activity)•1 ug/ml-10 ug/ml.

Increase SAC concentration for decreasing CD45RA expression.

GG uses 1:50,000 dilution.

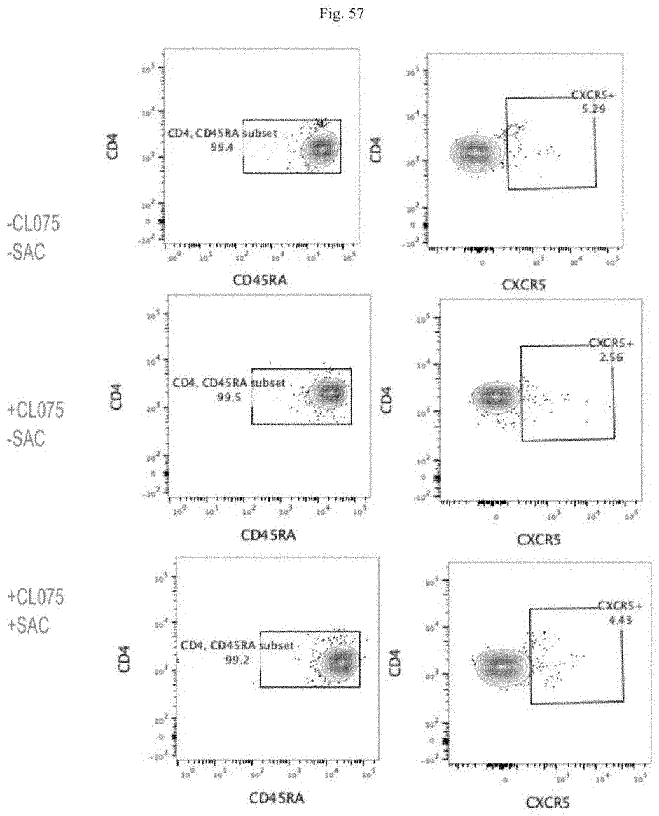

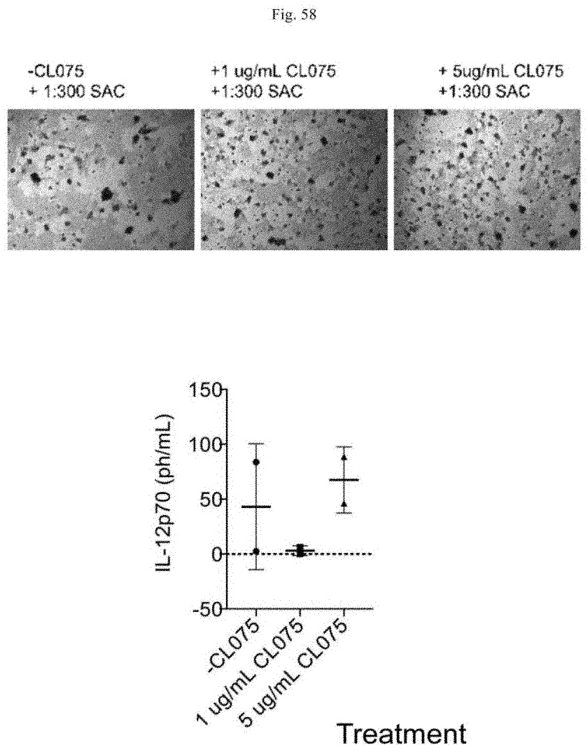

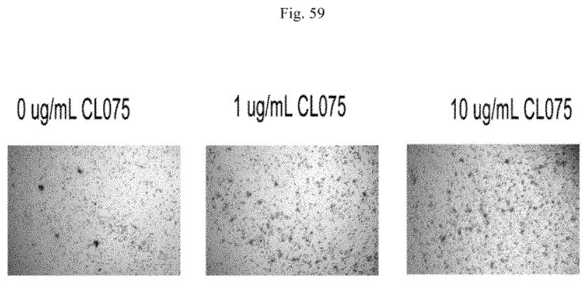

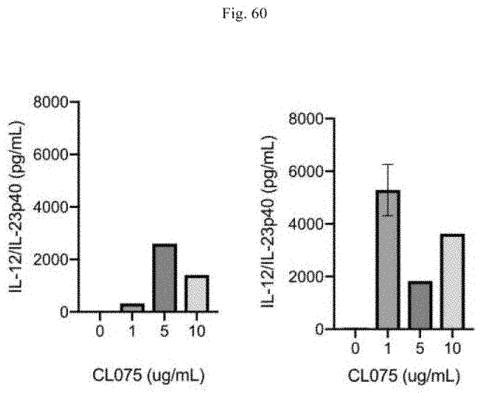

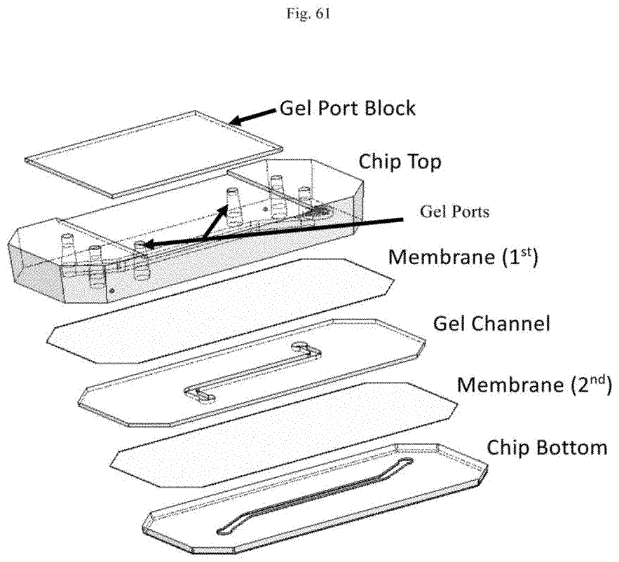

FIG. 57 shows an exemplary work flow using a 3 step procedure over 11 days including T follicular helper cells. Illustrations of several exemplary embodiments for substance testing are shown.
FIG. 58 shows exemplary flow cytometry staining of CD4, CD45RA, showing a purified CD4+CD45RA+ subset gated for showing percentages of CD4+CXCR5+ Follicular T cells. -CL075-SAC; +CL075-SAC; +CL075+SAC.
In one embodiment, T follicular helper cells shows Monocytes 24 Hours Post Activation.
FIG. 59 shows exemplary bright field micrographs comparing clustering of monocytes 24 Hours Post Activation in the presence of SAC in the presence of different concentrations of CL075, a thiazoloquinolone derivative that stimulates TLR8. Chart shows exemplary IL-12p70 secretion over low to high concentrations of CL075. 0 ug/mL CL075 1 ug/mL CL075 10 ug/mL CL075.
FIG. 60 shows exemplary bright field micrographs comparing clustering of monocytes 24 Hours Post Activation in the presence of SAC and GG in the presence of different concentrations of CL075, a thiazoloquinolone derivative that stimulates TLR8. MOI 1 and MOI 10 both yield the same amount of IL-12.
FIG. 61 shows exemplary IL-12/IL-23p40 secretion over low to high concentrations of CL075 comparing 2 modified methods.

B. Involvement of Immune Cell Clustering In Vitro, Mimicking Germinal Centers In Vivo.

Antigenic stimulation triggers specific B and T cells to move toward the T zone/follicle (T:B) border area of secondary lymphoid organs. There, B cells that present antigen-derived peptides to helper T cells become “authorized” to engage in a productive immune response. Successful B cells enter one of three developmental paths: they can differentiate into plasma cells (PCs) that secrete early, low-affinity antibody; they can re-establish a nonproliferative state and join the memory B cell pool; or they can enter the GC reaction. GCs appear several days after antigen exposure as clusters of rapidly proliferating cells in the center of B cell follicles. GCs comprise two anatomically defined areas: the dark zone (DZ), where cells proliferate and hypermutate their Ig genes, and the light zone (LZ), where antigen-driven selection takes place.

Following DZ hypermutation, B cells migrate to the LZ, where antigen is deposited as immune complexes on the surface of follicular dendritic cells (FDCs). LZ B cells compete to bind and retrieve antigen from FDCs and pre-sent it to GC-resident T follicular helper (Tfh) cells. B cells that have acquired higher affinity by virtue of SHM are more likely to receive positive selection signals, triggering their return to the DZ for further proliferation and hypermutation (cyclic re-entry, GC selection is thus reminiscent of Darwinian evolution: iterative cycles of descent with modification (SHM) followed by fitness (affinity)-based selection lead to increased fitness of the population as a whole. Sporadic differentiation of positively selected LZ B cells into PCs and memory B cells results in the progressive increase in the affinity of serum anti-bodies over time and upon re-immunization.

### III. Diagnostic Assays.

A one-size-fits-all treatment may well be reaching its end days as companies increasingly adopt approaches that involve biomarkers (there are now commercial databases that purport to track over 11,000 of them). Pharmacogenomic biomarkers in particular are used to create diagnostics that help to differentiate or stratify the likely outcomes a patient will experience with a drug, which can now be said to be targeted or tailored to patients with particular traits (i.e., personalized), leading to an era of so-called precision medicine. As more is understood about diseases and the why and how of their effects on people through advances in biomarkers and genomics, personalized medicine is becoming a natural result of biomedical science and a natural trajectory for the innovation-based biopharmaceutical industry.

There are several types of in vitro diagnostic assays contemplated for use as AB-Lymph-Node Chips. In general, diagnostic assays (e.g. complementary diagnostic that is not regulated by agencies such as the FDA, USA) are essential for the safe and effective use of therapeutics. Complementary diagnostics in general refers to tests used to improve disease management, early diagnosis, patient risk stratification and drug monitoring related to associated therapeutic class, but does not require a regulatory link to a specific therapeutic. Companion diagnostic refers to devices that provide information for the safe and effective use of a corresponding therapeutic product, typically linked to a specific drug within its approved labeling. In fact the FDA defines companion diagnostics as devices that provide information for the safe and effective use of a corresponding therapeutic product, typically linked to a specific drug within its approved labeling.

Further, diagnostic assays such as companion diagnostics may be required by governing agencies to be done which may be used to provide additional information, e.g., for improving the benefit/risk ratio, without restricting drug access, especially after a therapeutic is released by governing agencies for general use in patient populations or in specific patient populations are required.

In other words, the use of a complementary diagnostic is an assay that should be used but is not usually formally required before, during or after clinical trials, including used for treated patients in general populations while the use of a companion diagnostic is an assay that is required with before or after a therapeutic or other molecule is administered to subjects, or is ingested by subjects (e.g. as food or supplement, such as wheat products, vitamins, etc.) or comes in contact with subjects (e.g. cosmetics).

However both types of assays may be used during or after drug-diagnostic co-development. For one example, the same diagnostic may be used as both a complementary diagnostic and a companion diagnostic, depending upon the patient population for determining the chances of a patient to respond to a particular treatment. Merely as one illustrative example, a cancer patient that is a candidate for treatment using one of the engineered antibodies involving PD-L1/PD-1 is required to be tested for programmed death ligand-1 (PD-L1), (in some cases and/or PD-1) expression prior to treatment. However, when treating NSCLC, head and neck squamous cancer with another engineered antibody involving PD-L1/PD-1 clinical results observed are independent of PD-L1 expression level such that PD-L1 testing results do not inform or guide treatment decisions in these settings rendering PD-L1 testing as a complementary diagnostic. The threshold for PD-L1 positivity or negativity is that PD-L1 stained cell accounts for around 1% or more of tumor cells, or tumor and immune cells, assayed by immunohistochemistry staining methods. When PD-L1 testing is greater than 50%, then based on existing data, that the response rate is likely in the 40%-to-45% range. In contrast, PD-L1—negative patients have response rates between 20% to 30% depending on what platform is used.

Therefore, even when a patient does not show a biomarker characteristic(s) corresponding to a desired therapeutic response, there remains a possibility that a PD-L1— negative patient may receive a desired benefit of that treatment. Moreover, PD-L1 expression alone may not be enough to detect patients who will respond favorably.

Investigators used PD-L1 testing and next-generation sequencing (measuring microsatellite instability [MSI] and tumor mutational load [TML]) to profile 575 samples of gastric and gastroesophageal junction (G/GEJ) adenocarcinoma (Oncologist 2018 Apr. 27. [Epub ahead of print]). Depending on the cutoffs used, they found up to 64 patients (>10%) who had high MSI and TML and might benefit from checkpoint inhibitor therapy but would not be eligible based on the PD-L1 threshold required in the FDA labeling, the checkpoint inhibitor approved in this setting. “Are We Hitting the Mark With PD-L1 Biomarker Tests?” Diagnostics Aug. 15, 2018 (www.clinicaloncology.com/Diagnostics/Article/08-18/Are-We-Hitting-the-Mark-With-PD-L1-Biomarker-Tests-/52468). Thus, PD-L1 expression status alone is insufficient in determining which patients should be offered PD-1 or PD-L1 blockade therapy. Efficacy of PD-1 or PD-L1 inhibitors and PD-L1 expression status in cancer: meta-analysis BMJ 2018.

Therefore, there is a need for more accurate identification of patient populations using diagnostic assays so that more patients may be identified that are likely to derive benefits from certain types of therapeutics that are currently excluded based upon current diagnostic assays/biomarkers.

Thus, in some embodiments, an AB-Lymph node chip may be used to identify a biomarker, or lack of that biomarker, or an amount of a biomarker, or the presence of a particular allele or expression of a particular allele biomarker, which may either assist with identifying a patient or a patient subpopulation that should respond favorably, or at least assist with identifying patients who won't respond or are likely to have an adverse response at a level that is contemplated to use as a guide as to whether the presence of that biomarker or amount of the biomarker, as in a specific type of antibody production, would indicate using with caution or outright avoiding expose of that population to a particular drug.

In some embodiments, an AB-Lymph node chip may be used to supplement other types of diagnostic assays, including but not limited to diagnostic assays currently used for many type of therapeutics. In some embodiments, an AB-Lymph node chip may be used in comparison to other types of existing diagnostic assays, in part for determining whether an AB-Lymph node chip may provide more accurate information regarding the use of a known therapeutic.

In some embodiments, an AB-Lymph node chip may undergo validation testing for determining repeatability and accuracy of its technical performance. In some embodiments, an AB-Lymph node chip may undergo clinically related validation establishing that an AB-Lymph node chi acceptably identifies, measures, or predicts the concept of interest.

In some embodiments, an AB-Lymph node chip may have clinical utility for providing information that contributes to or a conclusion based upon that information alone that a given use of the test molecule will lead to a net improvement in health outcome or provide useful information towards a specific diagnosis, choice of treatment, choice of management, e.g. amount of therapeutic, number of repeat treatments, time between treatments, including whether it will prevent further disease symptoms or is use as a prophylactic treatment. Clinical utility includes the range of possible benefits or risks to individuals and populations.

Moreover, such AB-Lymph node chip may be used to provide information on patients as a prospective identification and use of any patient characteristic, including demographic, pathophysiologic, historical, genetic, and others, to select patients for a study or to analyze patient data to obtain a study population in which detection of a drug effect is more likely than it would be when used for a general unselected population.

As described herein, a variety of embodiments are contemplated and used for producing antibodies. In some embodiments, a single fluidic channel is opposed to a nonfluidic channel, wherein the nonfluidic channel contains a hydrogel is used. In some embodiments, a S-1 tall channel chip having 2 fluidic channels, wherein one channel contains a hydrogel is used. In some embodiments, a S-1 tall channel chip having 2 fluidic channels, wherein both channels contain hydrogels is used. In further embodiments, both channels contain the same type of hydrogel. In other further embodiments, each channel contains a different hydrogel.

Additional embodiments of microfluidic devices used herein are described in U.S. Pat. No. 8,647,861, (i.e. 861'), Organ Mimic Device With Microchannels And Methods Of Use And Manufacturing Thereof, hereby incorporated by reference in its entirety and in WO2018/017605 (605'), Human Lymphoid Tissue-On-Chip, hereby incorporated by reference in its entirety. 861' describes microfluidic “organ-on-chip” devices comprising living cells on membranes in microchannels exposed to culture fluid at a flow rate, including dual membrane microfluidic devices. 605' describes embodiments of organ-on-a-chip microfluidic device that specifically mimics a human lymph node and/or human lymphoid tissue.

### A. Types of Antibody Responses in Lymph Node Chips.

Human immune systems for producing antibodies has at least two arms: (1) an innate response, which reacts with high levels of antibody within minutes up to several days after antigenic stimulation, e.g. bacteria, viral, etc., followed by (2) an adaptive response, which reacts within one to two weeks for providing antibody production. Thus, for in vitro antibody testing both types of responses are desired in Lymph Node Chips.

When using immune cells, in particular B cells, derived from PBMCs, antibody responses in microfluidic devices described herein are recapitulating responses from circulating cells at the time of blood draw. These responses provide information on noncirculating antibody responses to antigens that are likely to be occurring in lymphatic tissue as opposed to measuring circulating antibodies directly from the serum of patients treated with the same antigens. It is known that the amount of antibodies to any antigen that are found in serum may differ in amounts and types depending upon variables such as age and season of the year.

In regards to diseases involving chronic inflammation, they are often considered different from each other with the perspective of the adaptive immune system, e.g. diseases in the gastrointestinal tract, Crohn's disease and ulcerative colitis. Thus driving treatments focused on each different type of disease. Current treatments for IBD, including Humira (adalimumab), which are monoclonal antibodies that act by suppressing the adaptive immune system. There is about a 50 percent chance of response and 35 percent chance of remission,” Gunn noted, and the drugs are also associated with significant side effects because they act by suppressing immune function. Qu Biologics “Developing CDx for Immunomodulator Drug to Treat Inflammatory Bowel Disease.” GenomeWeb, Mar. 23, 2017|Madeleine Johnson.

However, because same features of chronic inflammation may typically suppresses innate immune function, there may be an underlying dysregulation of the innate immune system present across a wide range of diseases.

Thus there are many challenges for determining whether an individual may respond to any particular antigen in light of estimates that to any one specific antigen (excluding superantigens), in part because there may be a reactive B cell that is 1 in a million or even down to 1 in 10 million, or less within a circulating white blood cell population.

B. In Vitro Antibody Assays Demonstrating at Least Three Types of Antibody Responses Related to Uses.

Although there is generally described two arms of an immune response, there are at least three types of antibody responses desired for observation in embodiments of Lymph Node chips of the present inventions. Thus, in some embodiments, an antibody response is sorted into one or more types of responses, by including but not limited to the use of biomarkers, i.e. cell surface markers, DNA markers, etc., cytokine responses, and the like.

Immediately after in vitro (mimicking in vivo) antigen stimulation, e.g. within a few minutes up to a few days, there may be an immediate response of B cells to produce IgG antibodies, albeit at low levels, e.g. picogram to nanogram levels. This immediate response is an indication that the white blood cell donor had prior exposure to this antigen resulting in memory B cells reactive to this antigen or has memory B cells produced by a prior exposure/reactivity to an antigen having cross reactivity to this in vitro antigen exposure. Thus in one embodiment, a lymph node chip is configured for identifying the presence of memory B cells that may react to an in vitro antigenic exposure under conditions allowing B cells to immediately produce antibodies, including but not limited to IgG. One example of a drug contemplated to induce this first type of antibody response is Adalimumab (HUMIRA®) (ADA), a recombinant a fully human anti-TNF-α IgG1 antibody intended for blocking the interaction of TNF-α with its receptors, is used in general for treating autoimmune diseases, often in active stages, such as Rheumatoid Arthritis (RA); Juvenile Idiopathic Arthritis (JIA); Psoriatic Arthritis (PsA); Plaque Psoriasis (Ps); Ankylosing Spondylitis (AS), where a well-known biomarker is an HLA-B27 allele; Crohn's Disease (CD); Ulcerative Colitis (UC).

In fact, a substantial proportion of patients with than patients without antibodies; rheumatoid arthritis (RA) do not respond, or lose initial response, to adalimumab treatment. One explanation for non-response is that patients develop anti-adalimumab antibodies which interfere with drug efficacy (neutralizing antibodies). Patients with antibodies showed less improvement in disease activity Serum antibodies against adalimumab are associated with lower serum adalimumab concentrations and non-response to adalimumab treatment. Ann Rheum Dis. 2007 July; 66(7): 921-926. Clinical response to adalimumab: relationship to anti-adalimumab antibodies and serum adalimumab concentrations in rheumatoid arthritis. Bartelds, Management of loss of response to anti-TNF drugs: Change the dose or change the drug? Gert Van Assche, et al. Journal of Crohn's and Colitis, Volume 2, Issue 4, December 2008, Pages 348-351.

These first type of antibodies may also be associated with or be a part of adverse events, merely as nonlimiting examples, Hypersensitivity: because anaphylaxis was reported following HUMIRA administration.

In some embodiments, comparisons are contemplated between antibodies produced in Lymph Node Chips induced by known drugs, e.g. Adalimumab vs. Bevacizumab.

A second type of antibody response develops over time after in vitro (mimicking in vivo) antigen stimulation of naive B cells recognizing that antigen under conditions resulting in affinity maturation over several days of proliferation. This second type of reaction would first show little Ig production, mainly as IgM, followed by an increasing amount of IgG becoming a high level antibody response following maturation of B cells into plasmablasts and plasma cells.

One example of a drug contemplated to induce this second type of antibody response is Bevacizumab (Avastin®), a recombinant humanised monoclonal antibody to vascular endothelial growth factor (VEGF) that is used in general for treating tumors. While it is generally accepted that few people react to Avastin, i.e., have adverse effects, in a small number of people adverse effects may include: Inflammation of the skin, Inflammation of the nose Nosebleeds, Rectal bleeding, GI perforation; Abnormal passage in the body. This type of passage—known as a fistula; Wounds that don't heal; Serious bleeding; Severe high blood pressure; Kidney problems; Infusion-related reactions such as a serious allergic reaction. Severe stroke or heart problems. These may include blood clots, Nervous system and vision problems.

Another example of a drug contemplated to induce this second type of antibody response is Adalimumab (HUMIRA®). Repeated exposures to this therapeutic is contemplated to mimic reactions of patients treated long term with multiple administration of adalimumab over time. In fact, the Food and Drug Administration (FDA) determined that additional safety testing was required after an analysis of spontaneous postmarketing adverse events, in particular for using higher amounts that in initial testing for use in patients having a moderately to severely active ulcerative colitis. BLA 125057/232. NDA 20725 PAIR 751-2: dated Sep. 28, 2012 with an external site posting dated April 2018.

These first type of antibodies may also be associated with or be a part of adverse events, merely as nonlimiting examples, Autoimmunity because treatment with HUMIRA may result in the formation of autoantibodies and, rarely, in development of a lupus-like syndrome. (www.humiraconnect.com/rheumatoid-arthritis/about/).

Therefore, in some embodiments, Adalimumab is contemplated as a test substance and WBCs derived from patients before, during and after treatment in antibody producing devices is contemplated for use in identifying associations between antibody production with genetic mutations or other biomarkers that predispose these patients to developing Hepatosplenic T-Cell Lymphoma (HSTCL).

When such an association is considered strong enough to indicate a potential safety concern, this information will be used for making decisions for that patient, or for new patients undergoing pretreatment evaluation on whether to initiate treatment, and if so, at which amount and for how long.

In some embodiments, Adalimumab is contemplated as a test substance and WBCs derived from patients before, during and after treatment in antibody producing devices is contemplated for use in evaluate effects of withdrawal and re-treatment with adalimumab and “switching” with other tumor necrosis factor (TNF)-blockers or biologics.

In some embodiments, Adalimumab used is contemplated as a test substance and WBCs derived from patients before, during and after treatment in antibody producing devices is contemplated for use in detecting a doubling of the risk of lymphoma events. In some embodiments, Adalimumab is contemplated as a test substance and WBCs derived from patients before, during and after treatment in antibody producing devices is contemplated for use in developing a validated anti-adalimumab antibody (AAA) assays, e.g. an assay having low sensitivity to product interference. In some contemplated embodiments, include data demonstrating that the assay is specific, sensitive and reproducible, and capable of sensitively detecting AAA responses in the presence of adalimumab levels that are expected to be present at the time of patient sampling. In some embodiments, Adalimumab is contemplated as a test substance for providing a validated AAA assay for use in measuring and analyzing the immunogenicity profile based on post-dose patient samples from a completed study, in part for evaluating the safety of induction regimens of adalimumab at doses higher than 160/80 mg. In this assay, the efficacy of Humira (adalimumab) can be assessed, both during induction treatment as well as during continued treatment after induction, and pharmacokinetic measurements should be conducted for exposure-response analysis. It would be desired to evaluate PBMCs at the time of loss of clinical remission in patients whose physicians plan to escalate the dose (e.g., decrease the dosing interval to weekly or increase the dosage) in response to loss of remission in part to determine whether patients who have low adalimumab exposures might benefit from dose escalation without increasing risk of serious adverse events. In some contemplated embodiments, evaluate the efficacy, safety and pharmacokinetics of adalimumab in pediatric patients 5 to 17 years of age with moderately to severely active ulcerative colitis, during induction treatment as well as during continued treatment after induction, and pharmacokinetic measurements should be conducted for exposure-response analysis.

In some embodiments, comparisons are made between antibodies produced in Lymph Node Chips induced by known drugs, e.g. Adalimumab vs. Bevacizumab.

A third antibody type of response during in vitro (mimicking in vivo) of antigen stimulation that results from de novo memory B cells that are present. De novo B cell memory in turn results from B cells that randomly developed a new type of antigenic recognition during DNA rearrangements while undergoing affinity maturation after stimulation by a different antigen molecule. This new type of antigenic recognition (i.e. new epitope) may bind to a test antigen and produce an antibody response that appears to be a memory response to that antigen, even though this is the first time the B cell population was exposed to that particular test antigen. In the context of testing antigens for use in safety testing, as described herein, there is a need for testing for this type of response for patients presumed to not have a particular adverse response to a drug merely because that patient was not previously treated with that drug.

One example of a drug contemplated to induce this third type of antibody response is Adalimumab (HUMIRA®), especially in light of clinical results where

Antidrug antibodies were reported for patients treated with Adalimumab over several years. After 3 years of treatment 28% of the patients (secondary failures); in 67% of cases these antidrug antibodies against Adalimumab developed with the first 6 months.

While it appeared that the majority of these antibodies were considered neutralizing antibodies, a recent discovery was that the development of anti-A dalimumab antibodies is associated with thromboembolic events. These discoveries point to the need for monitoring patients for development of antidrug antibodies to Adalimumab in additional to other biologic agents. Such monitoring is contemplated to be a safety strategy to predict both a potential lack of efficacy in addition to preventing toxicity such as adverse events, e.g. thromboembolic events, Deep vein thrombosis. Chapter 71, Kelley and Firestein's Textbook of Rheumatology E-Book, Firestein et al., 10^{th} Edition. However, although a subsequent study appeared to rule out this association at least one case study reported that anti-Adalimumab antibodies were associated with a new hyper coagulative state and occurrence of DVT (journal.chestnet.org/article/S0012-3692(19)32249-4/pdf).Adalimumab (Humira) and the risk of recurrent venous thromboembolism. Chinmaya Sharma, October 2019 Volume 156, Issue 4, Supplement, Page A780. In fact, during post marketing clinical use on larger patient populations, enough adverse events were observed for the FDA to issue new safety tests for the use of Adalimumab. See above embodiments for contemplated examples of how a microfluidic device of the preset inventions may be used for additional safety tests during use of a drug.

A type of third antibody response is contemplated to be associated with switching from an IgG1 response to an IgG4 response. Formation of anti-drug antibodies (ADAbs) not only affects the efficacy of biologic agents but also increases the risk of some adverse events. For adalimumab, the proportion of ADAb-positive patients ranged from <1% to 87%, but was most often around 0% Anti-adalimumab antibodies were detected in 46 patients (20%). Immunogenicity of biologic agents in rheumatoid arthritis patients: lessons for clinical practice. Thierry Schaeverbeke, Rheumatology, Volume 55, Issue 2, February 2016, Pages 210-220, Published: August 2015.

In some embodiments, comparisons are made in parallel between antibodies produced in Lymph Node Chips induced by known drugs, e.g. Adalimumab vs. Bevacizumab.

In another example, disease-modifying anti-rheumatic drugs (DMARDs) and anti-tumor necrosis factor-alpha (anti-TNF-α) agents are considered the treatments of choice for Rheumatoid arthritis (RA) patients. In some cases, when patients fail on DMARD therapy, one recommended therapeutic alternative is anti-TNF-α therapy, e.g. (adalimumab) Another suggested therapy is using an interleukin 1 (IL-1) receptor antagonists. Methotrexate (MTX) may be used in combination with these antibody therapies. Clinical trials, however, indicate that a significant number of RA patients do not respond to these therapies. Thus, an optimal therapeutic strategy for RA patients needs to be defined. Hyrich, et al., “Outcomes after switching from one anti-tumor necrosis factor alpha agent to a second anti-tumor necrosis factor alpha agent in patients with rheumatoid arthritis: results from a large UK national cohort study. Arthritis Rheum 2007; 56: 13-20). Patients receiving one type of anti-TNFalpha engineered antibodies are more likely to discontinue therapy because of side effects, which are occasionally severe, as well as infections and infusion reactions. However, even with these side effects, these types of therapies were determined to have a relatively acceptable toxicity profile.

The ACR (American College of Rheumatology) Criteria is a standard criteria to measure the effectiveness of various arthritis medications or treatments in clinical trials for Rheumatoid Arthritis. Clinical trials report the percentage of study participants who achieved ACR20, ACR50, and ACR70. For example, if a study reported that 55 percent of patients achieved ACR20, that means 55 percent of patients in the study achieved a 20 percent improvement in tender or swollen joint counts, as well as 20 percent improvement in three of the other five criteria.

If a clinical trial reports that 40 percent of patients achieved ACR50, that means 40 percent of patients in the study achieved a 50 percent improvement in tender or swollen joint counts, as well as 50 percent improvement in three of the other five criteria. The same applies to ACR70, only with a 70 percent improvement level. For patients to be assessed using ACR criteria, they must have completed the clinical trial. https://www.verywellhealth.com/acr-american-college-of-rheumatology-criteria-190531 discrete time points (usually baseline and post-baseline comparison).

ACR20 is ≥20% improvement.

ACR50 is ≥50% improvement.
ACR50 responders include ACR20 responders.

ACR70 is ≥70% improvement.
ACR70 responders include ACR20 & ACR50 responders.

Thus, in some embodiments, antibody producing devices described herein are seeded with autologous cells derived from arthritis patients before, during and after treatment. In some embodiments, antibody measurements, including but not limited to amounts of isotype classes, e.g. IgM, IgG, IgE, etc., subclasses, e.g. IgG1, IgG2, IgG4, etc., and avidity to the test drug, e.g., amount of antibodies binding drug molecules under a range of salt conditions contemplated to separate low, medium and high avidity binding for determine amounts of binding antibodies within each avidity range.

Switchers refer to patients treated with one type of molecule followed by treatment with a molecule targeting a similar pathway or molecule, such as two anti-TNF-alpha molecules. Thus, switchers treated with first with adalimumab anti-TNF antibody more frequently develop antibodies against a second engineered anti-TNF antibody than anti-TNF naive patients, referring to patients who've not received an anti-TNF treatment. It was observed that when a response to a second engineered anti-TNF antibody was limited in switchers without anti-adalimumab antibodies, which raised the question as to whether a second anti-TNF therapy should be offered to RA-patients who fail on initial treatment with anti-TNF adalimumab, in the absence of anti-biological antibodies. Bartelds 2009.

Thus some embodiments, comparisons are made between antibodies produced in Lymph Node Chips induced by known drugs having a similar mode of actions, i.e. an anti-TNF-alpha antibody, e.g. Adalimumab followed by another round of stimulation using another anti-TNF antibody, and vice versus.

B. Examples of Substance Testing in Microfluidic Devices.

In some embodiments, test substances are added to B cell stimulation media, replacing the IgM/IgG. It is not intended to limit the test antigen to any particular type of molecule or compound. It is not intended to limit the test antigen to known antigens. Indeed, in some embodiments, any type of compound known to contact humans is intended for use. Merely as one example, a test compound is CpG, intended to mimick bacterial antigenic DNA containing—CpG motifs. CpG oligodeoxynucleotides (or CpG ODN) refer to short single-stranded synthetic DNA molecules that contain a cytosine triphosphate deoxynucleotide (“C”) followed by a guanine triphosphate deoxynucleotide (“G”).

Test substances include but are not limited to drug candidates, drug therapeutics at any stage of preclinical and clinical testing, e.g. proteins, peptides, small molecules, large molecules, antibodies, glycoproteins, cytokines, chemokines, nucleic acids, vaccines, immune modulators, etc., for including in therapeutic cosmetics; and in general test molecules include but are not limited to antibodies (such as immunoglobulin treatments), glycoproteins, cytokines, proteins, peptides, small molecules, large molecules, nucleic acids, bacterial antigens, viral antigens, microbial antigens, indigents intended for a cosmetic, etc. Examples include influenza vaccines because they are apparently currently tested in ferrets and show 30-60% protection in humans.

Readouts include but are not limited to amounts or levels of soluble IgM and soluble IgG; amounts of levels of specific isotypes and/or subclasses of immunoglobulin; amounts or levels of affinity of antigen specific antibodies, for one example, establishing low, medium and high levels of affinity for particular test antigens, e.g. as part of an analysis of antigen-specific B-cell responses, i.e. useful for the detection of Anti-Drug specific Antibodies (ADAs), in some embodiments such affinity testing is contemplated to include measuring cross-reactive antibodies to molecules (e.g. another test molecule) that was not the actual test compound used for the initial stimulation of antibody production; cell proliferation rates; percentages of specific stages of differentiated B cells; percentages of live vs. dead cells during antibody production protocols; percentages of types of live vs. dead cells during antibody production protocols; such live vs. dead cell testing is contemplated for continuing beyond a 10-day culture time period; cell proliferation rates, etc.

In some embodiments, readout methods include but are not limited to Microscopy/Imaging; High content imaging, Flow cytometric characterization of cells and their surface markers, live vs. dead cell populations, sorting of post-stimulated cell populations; micro-organoid (i.e. cell clustering mimicking germinal centers) and (antigen-specific) plasma cell formation by histology/immuno-histochemical staining; Cytokine/Chemokine secretion profiles, Genomics and proteomics of at least one of stimulated, differentiating and cells undergoing maintenance.

C. Exemplary Companion Assays.

In order to predict unwanted immunogenicity reactions in patients (e.g. neutralizing antibody formation, sensitization) of drug candidates early in drug development. In addition, it can also be used for efficacy assessment e.g. of vaccine candidates to select the most promising lead candidates.

In some embodiments, readouts are used for detecting potential immunogenicity issues of compounds early for contributing to decisions on whether to continue developing that particular compound for use as a therapeutic treatment.

Early in the research and development process, researchers can select for molecules that have low immunogenicity. Later, as the manufacturing process changes to increase the amount of product, a Lymph Node Chip as described herein, can ensure that the different process has not changed the immunogenicity of the product. Test batches of product before release into the market, as a quality control test. Thus, drug developers can use at all stages in the research, development, and manufacture of a biologic drug to monitor immunogenicity.

Find and understand molecular changes that increase or decrease immunogenicity. Measure relative immunogenicity of molecules produced with different processes. Compare immunogenicity with research product. Monitor batches for changes in immunogenicity, especially after any process changes. Reproduces human responses to immunosuppressants and immunopotentiators, in some cases where rodent and non-human primate studies did not.

In some embodiments, readouts are used for detecting potential immunogenicity issues of compounds early for contributing to decisions on whether to continue developing that particular compound for use as a therapeutic treatment.

### Validate the Assay.

Run controls through the system and evaluate whether they recapitulate preclinical results using other immunogenicity assay (e.g. ‘Major Histocompatibility Complex-Associated Peptide Proteomics’ (MAPPs)), in particular for Avastin (considered to have low Immunogenicity in a human population) and Humira (considered to have High Immunogenicity in a human population).

D. Exemplary Diagnostic Assays.

A patient population created by a similar diagnoses, then treated using the same pharmacological intervention, contains subpopulations of individuals who respond differently, with great variability in both efficacy and safety outcomes.

A complementary diagnostic is a test that aids in the benefit-risk decision-making about the use of the therapeutic product, where the difference in benefit-risk is clinically meaningful.

Unfortunately, the majority of drug prescriptions distributed for many diseases and autoimmune disorders, including severe chronic diseases, are largely based on ‘trial and error’, i.e. prescribing a drug other people may respond to, or testing a drug under off label clinical use just to see if it works, and tragically not on solid biomarker data correlated with individuals who may respond favorably (or conversely biomarker data correlated with adverse responses).

Companion diagnostics (CDx) in general refers to assays for identifying safety and/or efficacy concerns related to administering compounds to subjects, including patients.

Companion diagnostic assay is an in vitro diagnostic device that provides information that is essential for the safe and effective use of a corresponding therapeutic product.

A companion diagnostic device can be in vitro diagnostic device or an imaging tool that provides information that is essential for the safe and effective use of a corresponding therapeutic product. When a diagnostic test is inaccurate, then the treatment decision based on that test may not be optimal. https://www.fda.gov/medical-devices/vitro-diagnostics/companion-diagnostics.

Thus, predictive biomarker assays need to be developed to guide the use of targeted therapies, specifically for identifying 1) preclinical drugs that may be safely administered to people in clinical trials; 2) identifying biomarkers correlated with adverse reactions and/or predicating efficacy; and 3) identifying biomarkers correlated with adverse reactions and/or predicating efficacy.
identify patients who are most likely to benefit from a particular therapeutic product;
identify patients likely to be at increased risk for serious side effects as a result of treatment with a particular therapeutic product; or
monitor response to treatment with a particular therapeutic product for the purpose of adjusting treatment to achieve improved safety or effectiveness.

Thus, companion diagnostics are developed and used in parallel to a compound intended for use in humans using a drug-diagnostic co-development model.

In some embodiments, a population of people of at least 30 different PBMC donors are tested with the same test substance in an AB Lymph Node Chip as described herein.

Results are used for determining whether a preclinical common assay, such as a complementary assay is enough for safety testing or whether a companion diagnostic is indicated. Such determinations are similar to results from a TCB T cell known assay where out of at least 30 donors, T cell responses are used for determining whether a diagnostic assay, companion assay, is indicated, e.g. T cell response on 30/30 people. However, even with T cell responses, there is no reliable predictive assay for a response in a patient. In some embodiments, where no reactivity is observed in a T cell assay, B cells may still react.

In some embodiments, an AB lymph Node assay as described herein for use in testing B cells derived from Lupus patients. In some embodiments, an AB lymph Node assay as described herein is used for testing B cells derived from Lupus patients for reactions to CpG, e.g. hospital flares of LUPUS after infection.

In some embodiments, a B cell reaction to a test substance is desired in an AB lymph Node Chip, e.g. when immunogenicity is desired during and after vaccination, including screening different types of adjuvant.

In some embodiments, an AB lymph Node assay as described herein is contemplated for use in identify potential responders to Avastin. Avastin (bevacizumab) adjuvant therapy, is used for treating advanced cancers, advanced colon, breast, lung, and kidney cancers. In the past year, however, Phase III trials failed to show its potential in late-stage prostate, advanced stomach, or early-stage colon cancers. In some embodiments, an AB lymph Node assay as described herein is contemplated for use to monitor therapy results and disease progression. Such information might spare those patients who would not see a response to the drug from the side effects associated with it. The therapy has been linked to adverse arterial clots, heart attacks, stroke, and bowel perforations.

In some embodiments, an AB lymph Node assay as described herein is contemplated for use in investigating the use of Avastin in combination with docetaxel and prednisone in men with hormone-refractory prostate cancer (HRPC) who did not extend overall survival compared to chemotherapy and prednisone alone. In some embodiments, an AB lymph Node assay as described herein is contemplated for use in investigating the use of Avastin for reducing the risk of cancer recurrence.

In some embodiments, an AB lymph Node assay as described herein is contemplated for use in correlating B cell responses to gene-expression profile signatures to distinguish patients with partial response (PR) from those with stable disease (SD) and progressive disease (PD) among women treated for breast cancer with Avastin as a neoadjuvant followed by Avastin plus chemotherapy.

In some embodiments, an AB lymph Node assay as described herein is contemplated for use in associating angiogenic tumor markers and gene-expression profiles with B cell responses in patients undergoing multiple cycles of treatment. E.g. markers in the angiogenesis process that were associated with response to therapy were VEGF-A, the molecular target of Avastin; PDGFRs, the receptors of VEGF-A; and CD31, an endothelial cell-adhesion molecule whose expression may be modulated by VEGF-A. Patients with higher tumor VEGF-A, CD31, and PDGFR-13 expression in the tumor vasculature tended to be more likely to benefit from Avastin treatment plus chemotherapy.

In some embodiments, an AB lymph Node assay as described herein is contemplated for use in correlating B cell responses with biomarkers that were missed in other studies for predicting which patients may benefit most from Avastin-based treatment.

When a subset of patients is identified using an AB lymph Node Chip, that subset of patients may benefit, e.g. that group could be targeted in another trial.

In some embodiments, an AB lymph Node assay as described herein is contemplated for use in tests to predict who will benefit for other drugs, which target the EGFR receptor. In some embodiments, an AB lymph Node assay as described herein is contemplated for use in with patient subsets already identified by another biomarker, e.g. patients with tumors expressing a mutant form of the molecule respond to one particular treatment.

### Exemplary Antibody Responses for Safety Testing of Substances.

Exemplary Embodiments of Microfluidic Devices as Lymph Node Chips, e.g. Antibody Producing Devices.

As described herein, a variety of embodiments are contemplated and used for producing antibodies. In some embodiments, a single fluidic channel is opposed to a nonfluidic channel, wherein the nonfluidic channel contains a hydrogel is used. In some embodiments, a S-1 tall channel chip having 2 fluidic channels, wherein one channel contains a hydrogel is used. In some embodiments, a S-1 tall channel chip having 2 fluidic channels, wherein both channels contain hydrogels is used. In further embodiments, both channels contain the same type of hydrogel. In other further embodiments, each channel contains a different hydrogel.

Additional embodiments of microfluidic devices used herein are described in U.S. Pat. No. 8,647,861, (i.e. 861'), Organ Mimic Device With Microchannels And Methods Of Use And Manufacturing Thereof, hereby incorporated by reference in its entirety and in WO2018/017605 (605'), Human Lymphoid Tissue-On-Chip, hereby incorporated by reference in its entirety. 861' describes microfluidic “organ-on-chip” devices comprising living cells on membranes in microchannels exposed to culture fluid at a flow rate, including dual membrane microfluidic devices. 605' describes embodiments of organ-on-a-chip microfluidic device that specifically mimics a human lymph node and/or human lymphoid tissue.

Embodiments for Seeding immune cells in hydrogels as described herein.

Although PBMCs generally have 40-50% T lymphocytes and only 3-15% B lymphocytes, whole lymph nodes contain about 50-60% T lymphocytes and about 40-50% B lymphocytes. Thus, in some embodiments, the T and B lymphocytes can be provided within the matrix in a ratio of about 40:60 to about 60:40 T lymphocytes to B lymphocytes and, preferably, the ratio of T lymphocytes to B lymphocytes is about 60:40. In some embodiments, the density of the T and B lymphocytes within the matrix is seeded to be about 1×10⁸ to about 2×10⁸ cells per milliliter, but at least greater than 500,000 cells per milliliter.

In some embodiments, T lymphocytes and B lymphocytes are seeded into the matrix by flowing PBMCs through the fluid path. In some embodiments, T cell (e.g., CD3+ cells) and B lymphocytes (e.g., CD19+ cells) are seeded within the matrix in a ratio of about 40:60 to about 60:40 T lymphocytes to B lymphocytes. According to any one or more aspects disclosed herein, the density of the T and B lymphocytes within the matrix can be greater than 500,000 cells per milliliter. According to any one or more aspects disclosed herein, the density of the T and B lymphocytes within the matrix can be about 1×10⁸ to about 2×10⁸ cells per milliliter.

The T lymphocytes and B lymphocytes can be formulated or seeded into the matrix by including PBMCs, where the PBMCs include the T lymphocytes and B lymphocytes.

Cells from human tonsils or other surgically resected lymph nodes.

### II. Detailed Description of Microfluidic Devices and Methods of Use.

As described herein, a variety of embodiments are contemplated and used for producing antibodies. In some embodiments, a single fluidic channel is opposed to a nonfluidic channel, wherein the nonfluidic channel contains a hydrogel is used. In some embodiments, a S-1 tall channel chip having 2 fluidic channels, wherein one channel contains a hydrogel is used. In some embodiments, a S-1 tall channel chip having 2 fluidic channels, wherein both channels contain hydrogels is used. In further embodiments, both channels contain the same type of hydrogel. In other further embodiments, each channel contains a different hydrogel.

Additional embodiments of microfluidic devices used herein are described in U.S. Pat. No. 8,647,861, (i.e. 861'), Organ Mimic Device With Microchannels And Methods Of Use And Manufacturing Thereof, hereby incorporated by reference in its entirety and in WO2018/017605 (605'), Human Lymphoid Tissue-On-Chip, hereby incorporated by reference in its entirety. 861' describes microfluidic “organ-on-chip” devices comprising living cells on membranes in microchannels exposed to culture fluid at a flow rate, including dual membrane microfluidic devices. 605' describes embodiments of organ-on-a-chip microfluidic device that specifically mimics a human lymph node and/or human lymphoid tissue.

### Exemplary Chip Activation

A. Chip Activation (functionalization) Compounds

In one embodiment, bifunctional crosslinkers are used to attach one or more extracellular matrix (ECM) proteins. A variety of such crosslinkers are available commercially, including (but not limited to) the following compounds:

### ANB-NOS (N-5-azido-2-nitrobenzoyloxysuccinimide)

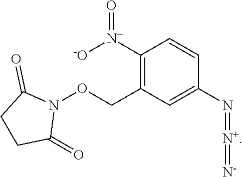

### Sulfo-SAND (sulfosuccinimidyl 2-[m-azido-o-nitrobenzamido]ethyl-1, 3→-dithiopropionate)

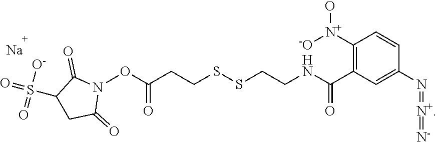

### SANPAH (N-succinimidyl-6-[4′-azido-2′-nitrophenylamino]hexanoate)

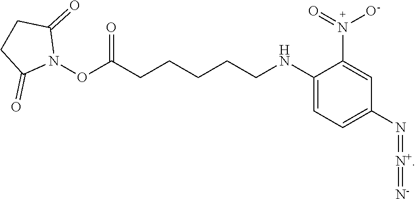

### Sulfo-SANPAH (“ER1”) (sulfosuccinimidyl-6-[4″-azido-2′-nitrophenylamino]hexanoate)

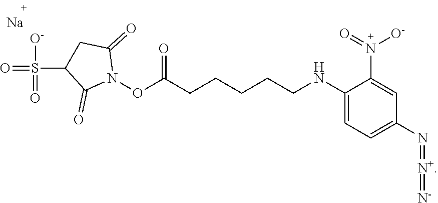

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH (“ER1”) should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

In some embodiments, APTES is used.

B. Exemplary Methods of Chip Activation.
1. Prepare and sanitize hood working space.
2. S-1 Chip (Tall Channel) Handling— Use aseptic technique, hold Chip using Carrier.
   a. Use 70% ethanol spray and wipe the exterior of Chip package prior to bringing into hood.
   b. Open package inside hood.
   c. Remove Chip and place in sterile Petri dish (6 Chips/Dish).
   d. Label Chips and Dish with respective condition and Lot #.
      3. Surface Activation with Chip Activation Compound (light and time sensitive).
a. Turn off light in biosafety hood.
b. Allow vial of Chip Activation Compound powder to fully equilibrate to ambient temperature (to prevent condensation inside the storage container, as reagent is moisture sensitive).
c. Reconstitute the Chip Activation Compound powder with ER-2 solution.
i. Add 10 ml Buffer, such as HEPES, into a 15 ml conical covered with foil.
ii. Take 1 ml Buffer from above conical and add to chip Activation Compound (5 mg) bottle, pipette up and down to mix thoroughly and transfer to same conical.
iii. Repeat 3-5 times until chip Activation Compound is fully mixed.
iv. NOTE: Chip Activation Compound is single use only, discard immediately after finishing Chip activation, solution cannot be reused.
d. Wash channels.
   i. Inject 200 ul of 70% ethanol into each channel and aspirate to remove all fluid from both channels.
   ii. Inject 200 ul of Cell Culture Grade Water into each channel and aspirate to remove all fluid from both channels.
   iii. Inject 200 ul of Buffer into each channel and aspirate to remove fluid from both channels.
e. Inject Chip Activation Compound Solution (in buffer) in both channels.
   i. Use a P200 and pipette 200 ul to inject Chip Activation Compound/Buffer into each channel of each chip (200 ul should fill about 3 Chips (Both Channels)).
   ii. Inspect channels by eye to be sure no bubbles are present. If bubbles are present, flush channel with Chip Activation Compound/Buffer until bubbles have been removed.
f. UV light activation of Chip Activation Compound Place Chips into UV light box.
   i. UV light treat Chips for 20 min.
   ii. While the Chips are being treated, prepare ECM Solution.
   iii. After UV treatment, gently aspirate Chip Activation Compound/Buffer from channels
   via same ports until channels are free of solution.
   iv. Carefully wash with 200 ul of Buffer solution through both channels and aspirate to remove all fluid from both channels.
   v. Carefully wash with 200 ul of sterile DPBS through both channels.
   vi. Carefully aspirate PBS from channels and move on to: ECM-to-Chip.
   Exemplary ECM-to-Chip: Coating Chips with ECM

Extracellular Matrix (ECM) refers to, for non limiting examples, e.g. proteins such as collagen I, in particular bovine collagen I, in some embodiments rat tail collagen I, including commercially obtained collagen, e.g. FibriCol® a type I bovine atelocollagen solution protein derived from bovine hide, a mixture of Collagen I with other types of ECM molecules and proteins, e.g., Matrigel® (BD Corning), laminin and Fibronectin; organ-specific extracellular matrix proteins; cell-specific extracellular matrix proteins; etc. Matrigel® (BD Corning) refers to a commercial reconstituted basement membrane extracted from Engelbreth-Holm-Swarm (EHS) mouse sarcoma cells/tumor. When the material is extracted from EHS tumors, it typically comprises laminin, collagen IV, entactin/nidogen, heparan sulfate proteoglycan (perlecan) and growth factors that occur naturally in EHS tumors. Examples for providing Matrigel directly from tumor cells is described in, Current Protocols in Cell Biology (1998) 10.2.1-10.2.9.

In some embodiments, chip channels are coated with ECM. In some embodiments, chip channels are partially filled with ECM. In some embodiments, chip channels are completely filled with ECM. ECM material may be diluted in Dulbecco's phosphate-buffered saline (DPBS) (without Ca²⁺, Mg²⁺).

A. Closed Top Microfluidic Chips without Gels.

In one embodiment, closed top antibody producing chips, or other type of organ-chip, do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips with Gels.

In one embodiment, closed top antibody producing chips-chips do contain gels, such as a gel layer, or bulk gel, including but not limited to a gel matrix, hydrogel, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

Additional embodiments are described herein that may be incorporated into closed top chips with gels.

C. Simulated Lumens.

In some embodiments, a simulated lumen is formed on chip. It is not meant to limit simulated lumens to a particular type of chip, indeed, both closed top and open top chips may be used, inducing examples of microfluidic devices described herein. Simulated lumens are not restricted to a particular structure, including but not limited to simulating follicles, simulating trabeculae simulating medullary cords, invaginations, providing folds and providing villi-like structures and villi. The term gels includes hydrogels.

A closed top antibody producing chips-chip comprising a gel-lined simulated lumen may be used for generating a more physiological relevant model of lymphoidal tissue. This, in some embodiments, closed top chips further comprise a gel simulated three-dimensional (3-D) lumen. In other words, a 3-D lumen may be formed using gels by providing simulated lymph node structures (e.g. as viscous fingers) and/or mimicking lymph nodules, including tonsils as a form of lymph node. In a preferred embodiment, the gel forms a lumen, i.e. by viscous fingering patterning.

In some embodiments, gels forming lumens may contain cell populations, e.g. WBS, subsets of WBCs, including T cells (CD3+), CD3+CD4+ T cells, B cells (CD3−CD19+), activated B cells (CD3−CD19+CD27+, etc., including B cells as described herein). In some embodiments, gels forming lumens may contain subsets of WBCs including monocytes, macrophages, follicular dendritic cells, etc. In some embodiments, lumens contain media as described herein. Conversely, lumens formed by hydrogels (with or without cells) may in turn be further filled with hydrogels, with the same or different concentration. These lumen filled hydrogels may in turn also contain WBS, subsets of WBCs, including T cells (CD3+), CD3+CD4+ T cells, B cells (CD3−CD19+), activated B cells (CD3−CD19+CD27+, etc., including B cells as described herein), monocytes, macrophages, etc.

Using viscous fingering techniques, e.g. viscous fingering patterning, a simulated lymph lumen may be formed by numerous simulated villi structures, e.g. as villi of cecal tonsils. Lymph villi (singular: villus) refer to small, finger-like projections that extend into the lymph tissue.

Viscous fingers may be short and thin, short and broad, or longer and thin, longer and broad, for mimicking different types of structures within lymph node tissue.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103, Author manuscript; available in PMC 2012 Jul. 16, herein incorporated by reference in its entirety. In one example of a viscous finger patterning method for use with microfluidic antibody producing chip, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated lymph structures.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular microenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid—liquid phase separation, etching of specific “block domains” in block co-polymers, phase separation to block-co-polymers, chemically cross-linked polymer networks with defined permeabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to these structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

D. Additional Embodiments of Microfluidic Devices.

It is not intended to limit antibody producing microfluidic devices to particular embodiments. In yet further embodiments, a microfluidic device is contemplated comprising a removable top of a closed top chip comprising a porous membrane (optionally stretchable) positioned in the middle over a microfluidic channel(s) further comprising structural anchors. In some embodiments, such a microfluidic device is contemplated comprising an open-top cavity. Structural anchors may be located on vertical wall surfaces. In some embodiments, structural anchors serve to prevent gel shrinkage-induced delamination.

E. Membrane Pores.

In some embodiments, membranes may be modified for particular pore numbers and sizes. Examples of embodiments for modifying membranes of microfluidic devices is provided in as shown in FIG. 4.

A master membrane mold 600 is preferably formed by patterning a photoresist to the desired shape and size on a silicon substrate. It should be noted that the posts 602 may be designed in any desired array depending on the intended design of the membrane 208. For example, the posts 602 may be arranged in a circular pattern to correspondingly form a circular patterned set of pores in the membrane 208. It should be noted that the posts 602 may have any other cross sectional shape other than pentagonal to make the corresponding pores in the membrane, as discussed above. It should also be noted that the master 600 may contain different height ridges to create non planar membranes.

Thereafter, as shown in FIG. 4B, the master 600 is preferably spin-coated with PDMS to form a spin coated layer 604. Thereafter, the spin-coated layer 604 is cured for a set time and temperature (e.g. 110° C. at 15 minutes) and peeled off the master 600 to produce a thin PDMS membrane 604 having the array of pentagonal through-holes 606, as shown in FIG. 4C. The example shown depicts fabrication of a 10 urn-thick PDMS membrane, although other thickness values are contemplated.

Although other materials may be used, PDMS has useful properties in biology in that it is a moderately stiff elastomer (1 MPa) which is non-toxic and is optically transparent to 300 nm. PDMS is intrinsically very hydrophobic, but can be converted to hydrophilic form by treatment with plasma. The membrane 604 may be engineered for a variety of purposes, some discussed herein. For example, the pores 606 on the membrane 604 may be coated or filled with ECM molecules or gels, such as Matrigel, collagen, laminin, fibronectin, fibrin, elastin, etc., which are known to those skilled in the art. A cellular interface may be provided by seeding and culturing one type of cell on one side of the membrane or different types of cells on each side of the membrane 604, as shown in FIG. 4D. In particular, as shown in FIG. 4D, one type of cells 608 are seeded on one side of the membrane 604 whereas another type of cell 610 is seeded on the opposing side of the membrane 604.

In some embodiments, pore sizes allow migration of cells from one side of the membrane to the other side. In some embodiments, pore sizes allow merely dendritic extensions of cells to extend to the other side of the membrane. In some embodiments, pore sizes merely allow diffusion of molecules from one side of the membrane to the other side. In some embodiments, pore sizes allow liquid hydrogels to flow through said pores for entering channels on the other side of the membrane. In some embodiments, pore sizes allow liquid hydrogels comprising cells to flow through said pores for entering channels on the other side of the membrane.

F. Incubation of Microfluidic Chips.

Antibody producing microfluidic chips are incubated at 37° C. in 5% Cot. In some embodiments, microfluidic chips are under constant fluid flow rates ranging from 1-100 ul per hour (hr). In some preferred embodiments, a flow rate is 15 uL/hr flow. In some preferred embodiments, a flow rate is 30 uL/hr flow. In some preferred embodiments, a flow rate is 60 uL/hr flow. In some preferred embodiments, a flow rate is 100 uL/hr flow. In some embodiments, after seeding chips with hydrogels containing white blood cells, chips are inserted into culture modules that are in turn inserted into and fluidically connected to reservoirs in perfusion manifolds. Fluid systems are regulated for cycle parameters, including removal of any bubbles. In some embodiments, systems are controlled using user interfaces, including Orb devices. Perfusion manifold assembly WO2014039514A2, Removing bubbles in microfluidic systems published. 2014 Mar. 13. Orb.

### EXPERIMENTAL

Isolate highly purified peripheral blood mononuclear cells (PBMCs) from fresh whole blood, buffy coat, bone marrow, cord blood, or leukapheresis products by negative selection. Red blood cells (RBCs), platelets and unwanted cells are targeted for removal with antibodies complexes and magnetic particles and separated using an EasySep™ magnet. Untouched PBMCs are simply collected into a new tube and are immediately available for downstream applications such as flow cytometry, culture or DNA/RNA extraction.

Magnetic separation—kit stem cells technology.
EasySep™ Direct Human PBMC Isolation Kit. Immunomagnetic negative selection from whole blood kit.
EasySep™ Human B Cell Isolation Kit. 9-Minute cell isolation kit using immunomagnetic negative selection.
EasySep™ Human T Cell Isolation Kit. 8-Minute cell isolation kit using immunomagnetic negative selection.
EasySep™ Direct Human Total Lymphocyte Isolation Kit. Immunomagnetic negative selection.
Exemplary experimental procedures:
1. Nonspecific antigen stimulation and differentiation of B cells in a 2D Culture (96 well plate).
2. Nonspecific antigen stimulation and differentiation of B cells in a 3D ECM (96 well plate).
3. Nonspecific antigen stimulation and differentiation of B cells in a 3D ECM in a S1 chip (Lymph Node-Chip).
4. Antigen specific stimulation and differentiation of B cells in a 3D ECM in aS1 chip (Lymph Node-Chip).
5. Nonspecific antigen stimulation and differentiation of B cells in a 3D ECM in a dual membrane (2 membrane 3 channel chip) (Lymph Node-Chip).
6. Antigen specific stimulation and differentiation of B cells in a 3D ECM in a dual membrane (2 membrane 3 channel chip).

In some embodiments, antibody levels are measured using a commercial Abcam simple step ELISA test.

Collagen Gel Labeling with NHS-Ester Dye.
1. To a formed a gel, add 10 ml of 50 mM borate buffer (pH 9.0) and incubate for 15 min at room temperature.
2. Meanwhile, calculate the amount of dye needed to properly label the amount of protein within the gel using the following equation:

((proteininmg)/(collagenMW))×(molarexcessofdye)×(dyeMW)×(1mgdyedilutioninμl)=dye volumeinμl15130000MW×2×981×200=45.48 μl.

The above equation is for 1 mg of Atto-488 NHS-ester diluted in 200 μl (5 mg/me of DMSO using a 2-molar excess which is recommended by the company.
Note: Each dye has a different molar-excess that works the best for NHS-conjugation. Do not assume the above will work for all dyes. Over labeling can lead to issues with gel formation later on.
Add 45.28 μl of Atto-488 NHS-ester dye to a 15 ml conical tube and bring the volume up to 5 ml with 50 mM borate buffer and vortex quickly.
Carefully aspirate the borate buffer from the tissue culture dish (bring the dish to a 45-degree angle and siphon off at the bottom edge with an aspiration pipette).
Add the dye solution to the collagen gel and wrap the culture dish with aluminum foil to protect from light. Allow the dye to conjugate to the collagen gel for 1 h at room temperature or 4 h at 4° C. (can do overnight) while rocking. Doyle, “Fluorescent Labeling of Rat-tail Collagen for 3D Fluorescence Imaging.”
Note: At room temperature the majority of the dye will conjugate within the first 20 min. Aspirate dye and add 10 ml of 50 mM Tris buffer (pH 7.5) to quench the dye reaction. Incubate with rocking for 10 min. Keep the gel covered with foil.
Add 10 ml of PBS⁺⁺. Rinse gel with PBS⁺⁺ 6× over the next 4 h to wash out the excess dye.

### Attachment of Gels to Surfaces

In one embodiment, adherence between the gel (including but not limited to a hydrogel) and a surface (including but not limited to the channel walls of a microfluidic device or chip) is desired to overcome loss of gel and cells over time. While a variety of chip designs could be used (including but not limited to 2 and 3 channel devices), for testing purposes, surfaces within a single channel chip were functionalized, i.e. coated, with one of the following exemplary materials: a bifunctional crosslinker (ER1); APTES (1% v/v); APTES (1% v/v)+glutaraldehyde (2.5% v/v); Polydopamine (1 mg/mL). The highest adherence between the gel and the channel walls was obtained using Polydopamine (1 mg/mL).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biochemistry, chemistry, microbiology, molecular biology, space biology, engineering and medicine, or related fields are intended to be within the scope of the following claims.

1. A method of activating B cells, comprising,
a) providing;
i) a microfluidic device comprising a space located in between an inlet and an outlet;
ii) a human cell population comprising B cells, said cells suspended in a hydrogel precursor; and
iii) a B cell activation medium, comprising a test substance and one or more activation associated molecules selected from the group consisting of IL-2, IL-21 and soluble CD40L molecules;
b) introducing said hydrogel precursor into said space;
c) treating said hydrogel precursor under conditions so as to at least partially solidify said hydrogel; and
d) flowing said B cell activation medium under conditions such that said B cells are exposed to said medium, wherein at least a portion of said B cells are activated to produce antibody.

2. The method of claim 1, further comprising:
e) collecting effluent from said outlet.

3. The method of claim 2, further comprising:
f) measuring the amount of antibodies in said effluent.

4. The method of claim 2, wherein said step e) occurs 1 to 10 days after step d).

5. The method of claim 1, wherein said test substance is selected from the group consisting of live bacteria, inactivated bacteria, bacterial spores, live virus, inactivated virus, live fungi, inactivated fungi and fungal spores.

6. The method of claim 1, wherein said test substance is selected from a drug, a vaccine, a cosmetic and a food substance.

7. The method of claim 1, wherein said test substance is an antigen selected from the group consisting of a bacterial antigen, a viral antigen and a fungal antigen.

8. The method of claim 1, wherein said antibodies comprise immunoglobulin M (IgM).

9. The method of claim 8, wherein said IgM is at a concentration of up to 40,000 ng/mL.

10. The method of claim 1, wherein said antibodies comprise immunoglobulin G (IgG).

11. The method of claim 10, wherein said IgG is at a concentration of up to 380,000 ng/mL.

12. The method of claim 1, wherein said B cells are exposed to said activation medium for 2-4 days.

13. The method of claim 1, wherein at least a portion of said activated B cells differentiate.

14. The method of claim 13, wherein said differentiating B cells comprise plasmablasts and plasma cells.

15. The method of claim 1, wherein said B cell activation medium contains only two activation associated molecules.

16. The method of claim 15, wherein said two activation associated molecules are IL-21 and soluble CD40L molecules.

17. The method of claim 16, wherein said soluble CD40L is provided by CD40L expressing feeder cells.

18. The method of claim 16, further comprising, after exposing B cells to said activation medium, exposing said B cells to a maintenance medium, said maintenance medium lacking IL-2 and soluble CD40L molecules.

19. The method of claim 18, wherein said maintenance medium exposure ranges from 2-5 days.

20. The method of claim 18, wherein said maintenance medium comprises molecules selected from the group consisting of IL-6, IL-21, and IFN-alpha.

21. The method of claim 1, wherein said B cells of step a) comprise memory B cells and naive B cells.

22. The method of claim 1, wherein said hydrogel comprises Matrigel® protein mixture.

23. The method of claim 1, wherein said hydrogel is a mixture of bovine collagen I and Matrigel® protein mixture.

24. The method of claim 1, wherein said human cell population is a Peripheral blood mononuclear cell (PBMC) population.

25. The method of claim 1, wherein said human cell population is an isolated tonsil white blood cell population.

26. The method of claim 1, wherein said human cell population is an isolated lymph node white blood cell population.

27. The method of claim 1, wherein said human cell population is a purified population of CD19+CD27+ B cells.

28. The method of claim 1, wherein said human cell population is a mixture of a purified population of CD19+CD27+ B cells and CD3+CD4+ T helper cells.

29. The method of claim 1, wherein said human cell population is a mixture of a purified population of CD19+CD27+ B cells and CD3+CD4+CXCR5(C-X-C Motif Chemokine Receptor 5)+ICOS(inducible T cell co-stimulator)+PD-1(programmed cell death-1)^{hi} T helper follicular cells.

30. The method of claim 1, wherein said flowing of said differentiation media is continuous flowing.

31. The method of claim 1, wherein said device fits into and is fluidically connected to a culture module that in turns fits into a perfusion manifold device.

32. The method of claim 1, wherein said test substance is an antibody or antibody fragment.

33. The method of claim 29, wherein said antibody is an anti-human antibody or antibody fragment.

34. The method of claim 27, wherein B cell populations was purified by negative selection.

35. The method of claim 1, wherein said microfluidic device comprises one or more gel ports and said hydrogel precursor is introduced into said space via said one or more gel ports.

36. The method of claim 35, further comprising, after step c) blocking said gel ports.

37. A microfluidic device comprising a space located in between an inlet and an outlet, said space comprising a human cell population comprising B cells, said cells suspended in a hydrogel precursor, said B cells exposed to a B cell activation medium, comprising a test substance and one or more activation associated molecules selected from the group consisting of IL-2, IL-21 and soluble CD40L molecules.

38. The device of claim 37, wherein said B cell activation medium lacks IL-2.

39. The device of claim 38, wherein said B cell activation medium contains IL-21 and soluble CD40L molecules.

40. The device of claim 39, wherein said soluble CD40L molecules are provided by CD40L expressing feeder cells.

41. A method of activating B cells, comprising,
a) providing;
i) a microfluidic device comprising a space located in between an inlet and an outlet;
ii) a human cell population comprising B cells, said cells suspended in a hydrogel precursor;
iii) a B cell maintenance medium lacking IL-2 and soluble CD40L molecules; and
iv) a B cell activation medium, comprising a test substance and one or more activation associated molecules selected from the group consisting of IL-2, IL-21 and soluble CD40L molecules;
b) introducing said hydrogel precursor into said space;
c) treating said hydrogel precursor under conditions so as to at least partially solidify said hydrogel;
d) flowing said B cell maintenance medium under conditions such that said B cells are exposed to said maintenance medium; and
e) flowing said B cell activation medium under conditions such that said B cells are exposed to said activation medium, wherein at least a portion of said B cells are activated to produce antibody.

42. The method of claim 41, further comprising:
f) collecting effluent from said outlet.

43. The method of claim 42, further comprising:
g) measuring the amount of antibody in said effluent.

44. The method of claim 41, wherein said B cells are exposed to said maintenance medium for more than one day.

45. The method of claim 41, wherein said maintenance medium comprises molecules selected from the group consisting of IL-6, IL-21, and IFN-alpha.

46. A method of providing a gradient within a microfluidic device, comprising, a) providing; i) a microfluidic device comprising a space located in between an inlet and an outlet; ii) a human cell population comprising B cells, said cells suspended in a hydrogel precursor; and iii) a B cell medium, comprising at least one chemokine, and one or more activation associated molecules selected from the group consisting of IL-2, IL-21 and soluble CD40L molecules; b) introducing said hydrogel precursor into said space; c) treating said hydrogel precursor under conditions so as to at least partially solidify said hydrogel; and d) flowing said B cell medium under conditions such that said B cells are exposed to said medium, wherein said chemokine in said medium form gradients having lower to higher concentration levels with said hydrogel, wherein at least a portion of said B cells are located in one level.

47. The method of claim 46, wherein at least a portion of said B cells located in one or more levels are stimulated to migrate into a different level in response to said chemokine.
